(19) United States
(12) Patent Application Publication
(10) Pub. No.: US 2015/0133434 A1
(43) Pub. Date: May 14, 2015

(54) Compositions and Methods for Reactivating Latent Immunodeficiency Virus

(71) Applicants: The J. David Gladstone Institutes, San Francisco, CA (US); Icahn School of Medicine at Mount Sinai, New York, NY (US)

(72) Inventors: Melanie Ott, Mill Valley, CA (US); Eric M. Verdin, Mill Valley, CA (US); Ming-Ming Zhou, Old Greenwich, CT (US)

(21) Appl. No.: 14/385,475

(22) PCT Filed: Mar. 13, 2013
(86) PCT No.: PCT/US13/30879

§ 371 Date: Sep. 15, 2014

Related U.S. Application Data
(60) Provisional application No. 61/616,764, filed on Mar. 28, 2012.

(51) Int. Cl. A61K 31/5517; A61K 31/4745; G01N 33/569; A61K 31/517; A61K 45/06; A61K 31/37; A61K 31/4184

(52) U.S. Cl. CPC A61K 31/5517; A61K 31/37; A61K 31/4745; A61K 31/4184; A61K 31/517; A61K 45/06; G01N 33/56988; G01N 2333/16
USPC 514/220; 514/457; 514/293; 514/387; 514/266.3; 435/375; 435/5

(57) ABSTRACT

The present disclosure provides compositions and methods for reactivating latent immunodeficiency vims. The methods generally involve contacting an HIV-infected cell in which HIV is latent with an agent that binds a bromodomain (BRD) in the cell. Latently infected cells contain replication-competent integrated HIV-1 genomes that are blocked at the transcriptional level, resulting in the absence of viral protein expression. The present disclosure provides methods for reducing the reservoir of latent immunodeficiency virus in an individual.

### CROSS-REFERENCE

This application claims the benefit of U.S. Provisional Patent Application No. 61/616,764, filed Mar. 28, 2012, which application is incorporated herein by reference in its entirety.

### STATEMENT REGARDING FEDERALLY SPONSORED RESEARCH

This invention was made with government support under Grant Nos. R01 AI083139, P01 GM066531, and R01 HG004508, awarded by the National Institutes of Health. The government has certain rights in the invention.

### BACKGROUND

Combination antiretroviral therapy can control HIV-1 replication and delay disease progression. However, despite the complete suppression of detectable viremia in many patients, viremia reemerges rapidly after interruption of treatment, consistent with the existence of a latent viral reservoir. This reservoir is thought to consist mainly of latently infected resting memory CD4⁺ T cells. Due to the long half-life of this reservoir (44 months), it has been estimated that its total eradication with current treatment would require over 60 years.

Latently infected cells contain replication-competent integrated HIV-1 genomes that are blocked at the transcriptional level, resulting in the absence of viral protein expression. HIV depends on both cellular and viral factors for efficient transcription of its genome, and the activity of the HIV promoter is tightly linked to the level of activation of its host cell.

Bromodomains are conserved sequence elements identified in several protein families and constitute chromatin targeting modules that mediate attachment to acetylated histones. Bromodomain-containing protein 4 (BRD4) is a member of the BET family of bromodomain proteins, which characteristically have two tandem N-terminal bromodomains followed by an extraterminal (ET) domain. BRD4 has been identified as an interaction partner with the positive transcription elongation factor b (P-TEFb) complex. Other bromodomain-containing proteins include CBP and PCAF.

### LITERATURE

U.S. Pat. No. 7,232,685; U.S. Pat. No. 7,544,467; WO 2009/020559; WO 2011/161031.

### SUMMARY

The present disclosure provides compositions and methods for reactivating latent immunodeficiency virus.

### BRIEF DESCRIPTION OF THE DRAWINGS

FIG. 1 depicts the effect of Compound 5110065 on the activity of the HIV promoter in the presence (HIV LTR+Tat) and absence (HIV LTR) of Tat. The experiments used Jurkat 1 G5 cells, which contain a stably integrated HIV LTR luciferase vector; and Jurkat 1 G5-Tat cells, which are further infected with a lentivirus expressing Tat. The concentration of compound was varied from 1.25 μM to 40 μM and luciferase activity was used as a measurement of HIV transcription. Numbers on X axis are in μM.

FIG. 2 depicts the effect of compound 6163501 on the activity of the HIV promoter in the presence (HIV LTR+Tat) and absence (HIV LTR) of Tat. Numbers on X axis are in μM.

FIG. 3 depicts the effect of compound 791084 on the activity of the HIV promoter in the presence (HIV LTR+Tat) and absence (HIV LTR) of Tat. Numbers on X axis are in μM.

FIG. 4 depicts the effect of compound 7910896 on the activity of the HIV promoter in the presence (HIV LTR+Tat) and absence (HIV LTR) of Tat. Numbers on X axis are in μM.

FIG. 5 depicts the effect of compound 5110065 on HIV expression as monitored by flow cytometry measurement (or FACS analysis) of GFP expressing cells. Comparative data is shown for A2 cells, (Jurkat cells contain a latent integrated LTR-Tat-IRES-GFP retroviral vector (Jordan et al. (2003) EMBO J. 22(8): 1868-1877)) and A72 cells (Jurkat cells containing an integrated LTR-GTP retroviral vector lacking Tat (Jordan et al. EMBO J. 2001, 20 1726-1738)). The concentration of compound was varied from 10 nM to 20 μM. The bottom panels depict the % live cells following treatment. The upper panels depict the % of those live cells that are GFP+. For the left panels, for each condition on the X axis, 4 bars are shown (from left to right: Control, 0.25 ng/ml TNFa, 0.5 ng/ml TNFa, and 1 ng/ml TNFa. For the right panels, for each condition on the X axis, 4 bars are shown (from left to right: Control, 0.5 ng/ml TNFa, 1 ng/ml TNFa, and 2.5 ng/ml TNFa.

FIG. 6 depicts the effect of compound 6163501 on HIV expression as monitored by flow cytometry measurement of GFP expressing cells. Comparative data are shown for A2 and A72 cells. For the left panels, for each condition on the X axis, 4 bars are shown (from left to right: Control, 0.25 ng/ml TNFa, 0.5 ng/ml TNFa, and 1 ng/ml TNFa. For the right panels, for each condition on the X axis, 4 bars are shown (from left to right: Control, 0.5 ng/ml TNFa, 1 ng/ml TNFa, and 2.5 ng/ml TNFa.

FIG. 7 depicts the effect of compound 7910894 on HIV expression as monitored by flow cytometry measurement of GFP expressing cells. Comparative data are shown for A2 and A72 cells. For the left panels, for each condition on the X axis, 4 bars are shown (from left to right: Control, 0.25 ng/ml TNFa, 0.5 ng/ml TNFa, and 1 ng/ml TNFa. For the upper right panel, for each condition on the X axis, 4 bars are shown (from left to right: Control, 0.5 ng/ml TNFa, 1 ng/ml TNFa, and 5 ng/ml TNFa. For the lower right panel, for each condition on the X axis, 4 bars are shown (from left to right: Control, 0.5 ng/ml TNFa, 1 ng/ml TNFa, and 2.5 ng/ml TNFa.

FIG. 8 depicts the effect of compound 129509 on HIV expression as monitored by flow cytometry measurement (of GFP expressing cells. Comparative data are shown for A2 and A72 cells. For the left panels, for each condition on the X axis, 4 bars are shown (from left to right: Control, 0.25 ng/ml TNFa, 0.5 ng/ml TNFa, and 1 ng/ml TNFa. For the upper right panel, for each condition on the X axis, 4 bars are shown (from left to right: Control, 0.5 ng/ml TNFa, 1 ng/ml TNFa, and 5 ng/ml TNFa. For the lower right panel, for each condition on the X axis, 4 bars are shown (from left to right: Control, 0.5 ng/ml TNFa, 1 ng/ml TNFa, and 2.5 ng/ml TNFa.

FIGS. 9A-C depict the effect of compound JQ1 on HIV expression in A2 cells as monitored by flow cytometry measurement of GFP expression. Panel (A) shows the % of live cells that are GFP+, Panel (B) demonstrates the level of HIV activation and Panel (C) indicates the % live cells. For all panels, for each condition on the X axis, 3 bars are shown (from left to right: Control, 0.1 ng/ml TNFa, 0.5 ng/ml TNFa.

FIGS. 10A-C depict the effect of compound JQ1 on HIV expression in A72 cells as monitored by flow cytometry measurement of GFP expression. Panel (A) shows the % of live cells that are GFP+, Panel (B) demonstrates the level of HIV activation and Panel (C) indicates the % live cells. For all panels, for each condition on the X axis, 4 bars are shown (from left to right: Control, 0.5 ng/ml TNFa, 1 ng/ml TNFa, and 5 ng/ml TNFa.

FIGS. 11A-C depict the effect of compound JQ1 on HIV expression in J-Lat 6.3 cells as monitored by flow cytometry measurement of GFP expression. Panel (A) shows the % of live cells that are GFP+, Panel (B) demonstrates the level of HIV activation and Panel (C) indicates the % live cells. For all panels, for each condition on the X axis, 4 bars are shown (from left to right: Control, 1 ng/ml TNFa, 2.5 ng/ml TNFa, and 5 ng/ml TNFa.

FIGS. 12A-C depict the effect of compound JQ1 on HIV expression in J-Lat 11.1 cells as monitored by flow cytometry measurement of GFP expression. Panel (A) shows the % of live cells that are GFP+, Panel (B) demonstrates the level of HIV activation and Panel (C) indicates the % live cells. For all panels, for each condition on the X axis, 5 bars are shown (from left to right: Control, 0.5 ng/ml TNFa, 1 ng/ml TNFa, 2.5 ng/ml TNFa, and 5 ng/ml TNFa.

FIGS. 13A-C depict the effect of compound JQ1 on HIV expression in J-Lat 5A8 cells as monitored by flow cytometry measurement of GFP expression. Panel (A) shows the % of live cells that are GFP+, Panel (B) demonstrates the level of HIV activation and Panel (C) indicates the % live cells. The 5A8 cells are responsive to CD3/CD28 activation and not TNF-alpha. For all panels, for each condition on the X axis, 3 bars are shown (from left to right: Control; 3 μg/ml CD3; and 3 μg/ml CD3 with 1 μg/ml CD28.

FIGS. 14A-C depict the effect of compounds 5110065, 7910894, and 7910896 on the activity of the HIV promoter in the presence (HIV LTR+Tat) and absence (HIV LTR) of Tat.

FIG. 15 depicts the effect of compound JQ1 on HIV expression in A2 and A72 cells. For the Left panels, for each condition on the X axis, 3 bars are shown (from left to right: Control; 0.1 ng/ml TNFα, and 0.5 ng/ml TNFα. For the Right panels, for each condition on the X axis, 4 bars are shown (from left to right: Control; 0.5 ng/ml TNFα, 1 ng/ml TNFα, and 5 ng/ml TNFα.

FIGS. 16A and 16B depict the binding affinity measurements of MS0124286 and MS0040472 to the bromodomains from the human proteins BRD4, CBP, and PCAF, as determined in a fluorescence anisotropy competition assay using a FITC-labelled MS417 as an assay probe.

FIGS. 17A-C depict features of the BRD4 bromodomain inhibitor MS147.

FIG. 18 depicts the chemical structures of various exemplary BRD inhibitors.

FIGS. 19A-Q provide amino acid sequences of various bromodomain-containing proteins. Bromodomains are depicted by bold text and underlining. FIG. 19A provides an amino acid sequence of a human BRD4 polypeptide (two bromodomains underlined and bolded); FIGS. 19B and 19C provide an amino acid sequence of a human CREB-binding protein (CRB); and FIG. 19D provides an amino acid sequence of a human p300/CBP-associated factor (PCAF) protein. FIGS. 19E-Q provide amino acid sequences of additional human BRD proteins (2, 3, 1, and 6-9). BRD2, BRD3, and BRD6 each have two bromodomains (underlined and bolded).

FIGS. 20A-B demonstrate that JQ1 activates latent HIV. All treatments on the X axis are in μM.

FIGS. 21A-B demonstrate that the JQ1 effect is Tat-independent. All treatments on the X axis are in μM.

FIGS. 22A-B demonstrate the reactivation of latent HIV with additional bromodomain-targeting compounds.

FIGS. 23A-B demonstrate the effect of bromodomain-targeting compounds in primary T-cell models of HIV latency.

FIGS. 24A-F demonstrate that JQ1 enhances transcription burst size.

FIGS. 25A-B demonstrate that the JQ1 effect in A72 cells is dependent on P-TEFb and BRD2. All treatments on the X axis are in μM.

FIG. 26 presents a schematic (model) illustrating that BET proteins restrict HIV transcription in the absence of Tat.

FIGS. 27A-B demonstrate that co-treatment with JQ1 and prostratin activates latent HIV. All treatments on the X axis are in μM.

FIG. 28 demonstrates the reactivation of latent HIV-1 by inhibition of BRD2 and BRD4. All treatments on the X axis are in μM.

### DEFINITIONS

The term “immunodeficiency virus” includes human immunodeficiency virus (HIV), feline immunodeficiency virus, and simian immunodeficiency virus. The term “human immunodeficiency virus” as used herein, refers to human immunodeficiency virus-1 (HIV-1); human immunodeficiency virus-2 (HIV-2); and any of a variety of HIV subtypes and quasispecies.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a compound that, when administered to a mammal or other subject for treating a disease, is sufficient to effect such treatment for the disease. The “therapeutically effective amount” will vary depending on the compound or the cell, the disease and its severity and the age, weight, etc., of the subject to be treated.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, a “pharmaceutical composition” is meant to encompass a composition suitable for administration to a subject, such as a mammal, especially a human. In general a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like. In some embodiments the composition is suitable for administration by a transdermal route, using a penetration enhancer other than dimethylsulfoxide (DMSO). In other embodiments, the pharmaceutical compositions are suitable for administration by a route other than transdermal administration. A pharmaceutical composition will in some embodiments include a subject compound and a pharmaceutically acceptable excipient. In some embodiments, a pharmaceutically acceptable excipient is other than DMSO.

As used herein, “pharmaceutically acceptable derivatives” of a compound of the invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and are either pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a bromodomain inhibitor” includes a plurality of such inhibitors and reference to “the HIV-infected cell” includes reference to one or more HIV-infected cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of reactivating latent HIV integrated into the genome of an HIV-infected cell. The methods generally involve contacting an HIV-infected cell in which HIV is latent with an agent that binds a bromodomain (BRD) in the cell. Latently infected cells contain replication-competent integrated HIV-1 genomes that are blocked at the transcriptional level, resulting in the absence of viral protein expression. The present disclosure provides methods for reducing the reservoir of latent immunodeficiency virus in an individual.

The present disclosure further provides detection methods for identifying a cell that has latent HIV. The methods generally involve contacting a cell obtained from an individual with a bromodomain inhibitor; and detecting expression of an HIV-encoded gene product. If the cell expresses an HIV-encoded gene product when contacted with the bromodomain inhibitor, but does not express detectable levels of the HIV-encoded gene product in the absence of the bromodomain inhibitor, the cell is considered to have latent HIV.

The present disclosure further provides a method of identifying a candidate agent for treating an HIV infection in an individual. The method generally involves contacting a primary cell identified using a subject method with a bromodomain inhibitor and a test agent; and determining the effect of the test agent on the level of HIV produced in the cell. A test agent that reduces the level of HIV produced in the cell, compared to the level of HIV produced in a control cell contacted with the bromodomain inhibitor but not with the test agent, is considered a candidate agent for inhibiting HIV and treating an HIV infection.

### Treatment Methods

The present disclosure provides methods for activating latent immunodeficiency virus in a cell, the methods generally involving contacting the cell with an agent that binds a bromodomain (BRD). The present disclosure provides methods for reducing the reservoir of latent immunodeficiency virus in an individual by administering to the individual an effective amount of an agent that binds a BRD in a cell latently infected with HIV. Suitable agents are described below. The present disclosure provides methods of treating an immunodeficiency virus infection in an individual, the methods generally involving co-administering to the individual an agent that reactivates latent HIV and an anti-HIV agent.

An “agent that binds a bromodomain” is generally a compound that binds a BRD and activates immunodeficiency virus transcription. An agent that binds a BRD and activates immunodeficiency virus transcription is also referred to herein as a “BRD inhibitor.” Without being bound to theory, a BRD inhibitor may act to activate latent HIV by inhibiting binding of cellular proteins, P-TEFb (including CDK9 and CycT1) to a bromodomain of a bromodomain-containing protein.

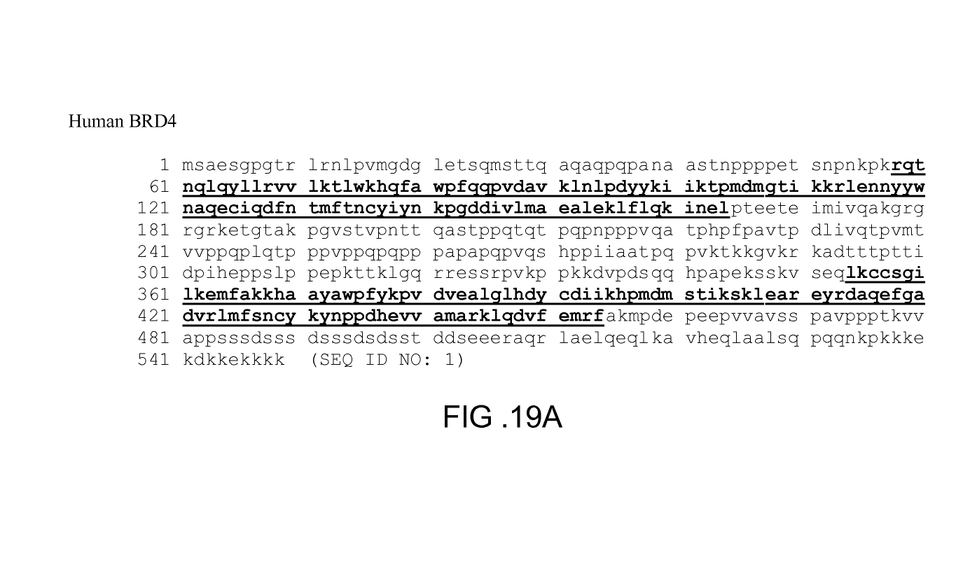

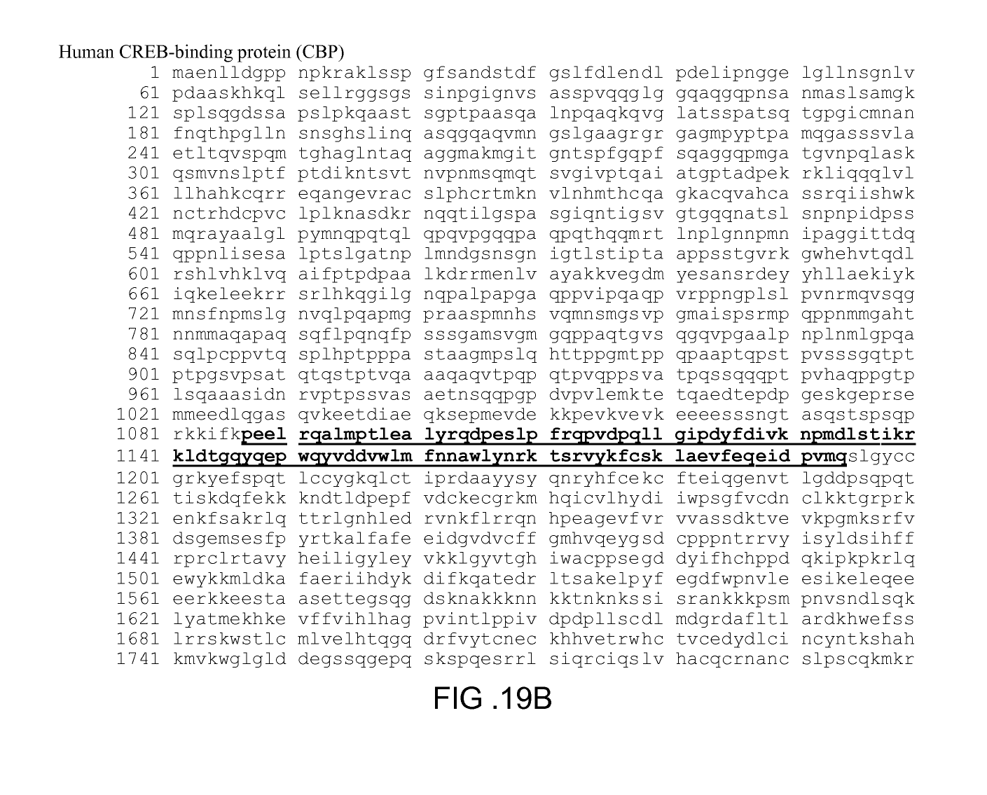

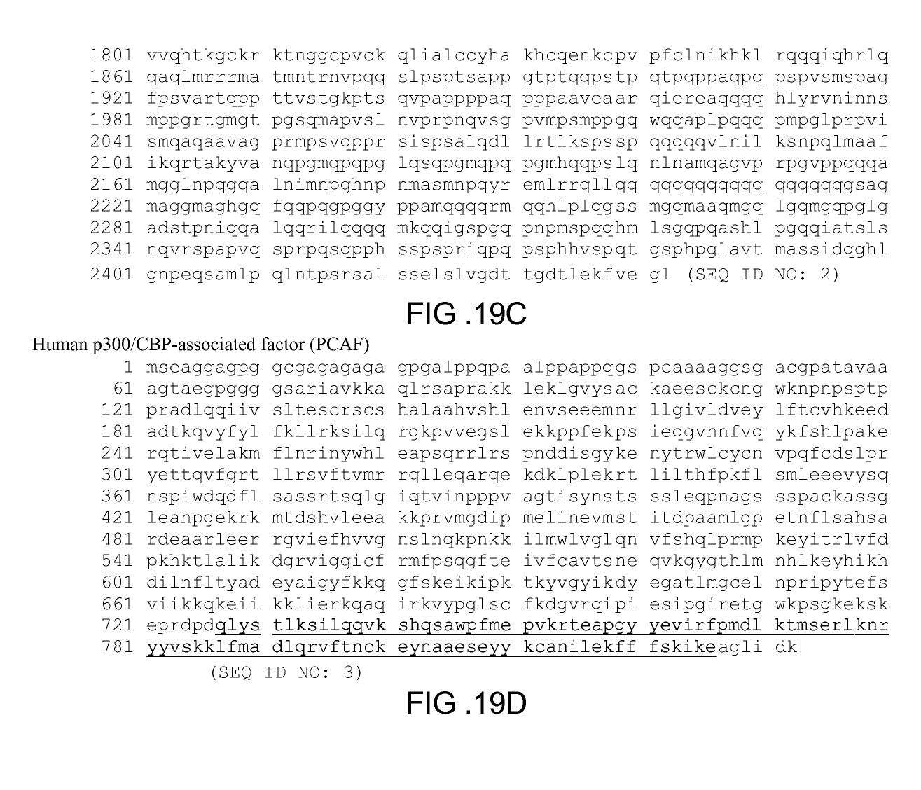

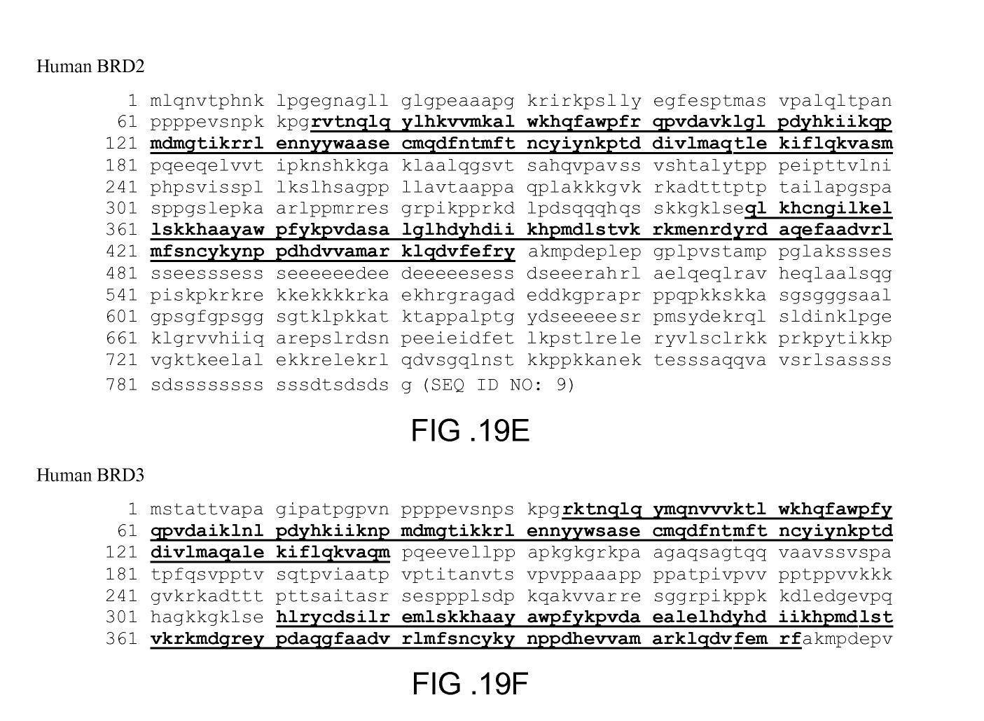

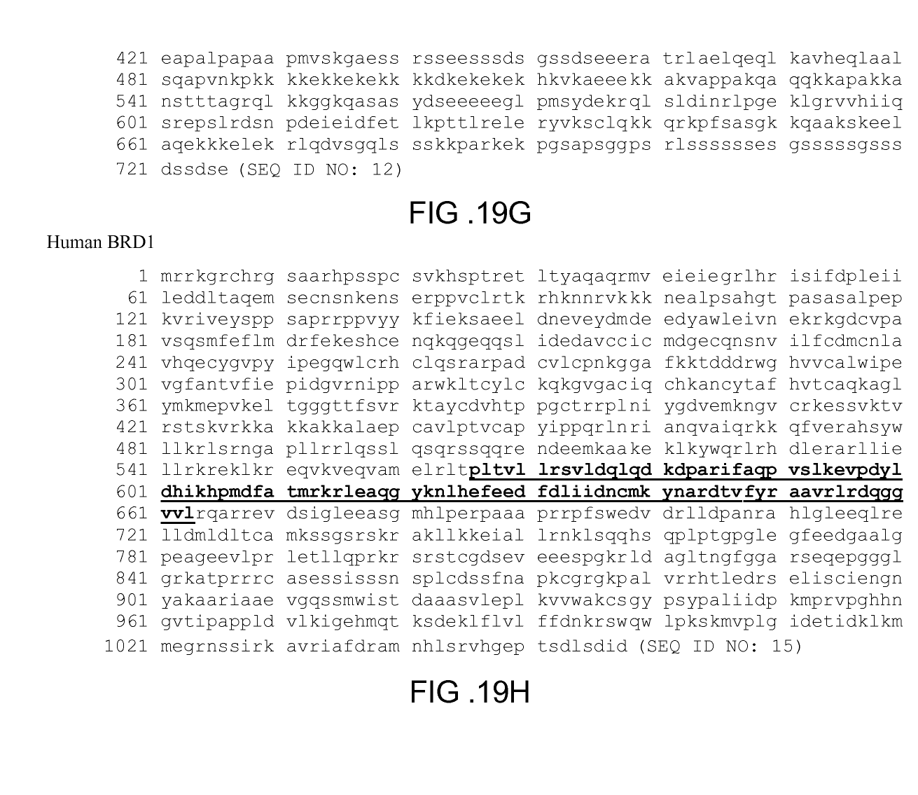

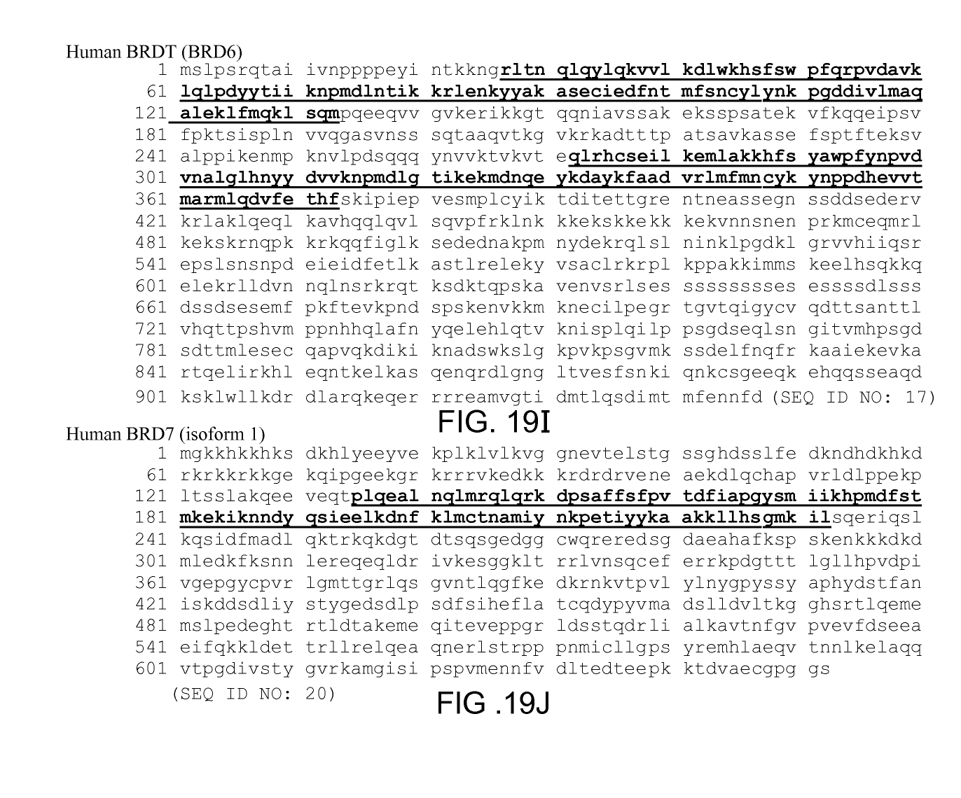

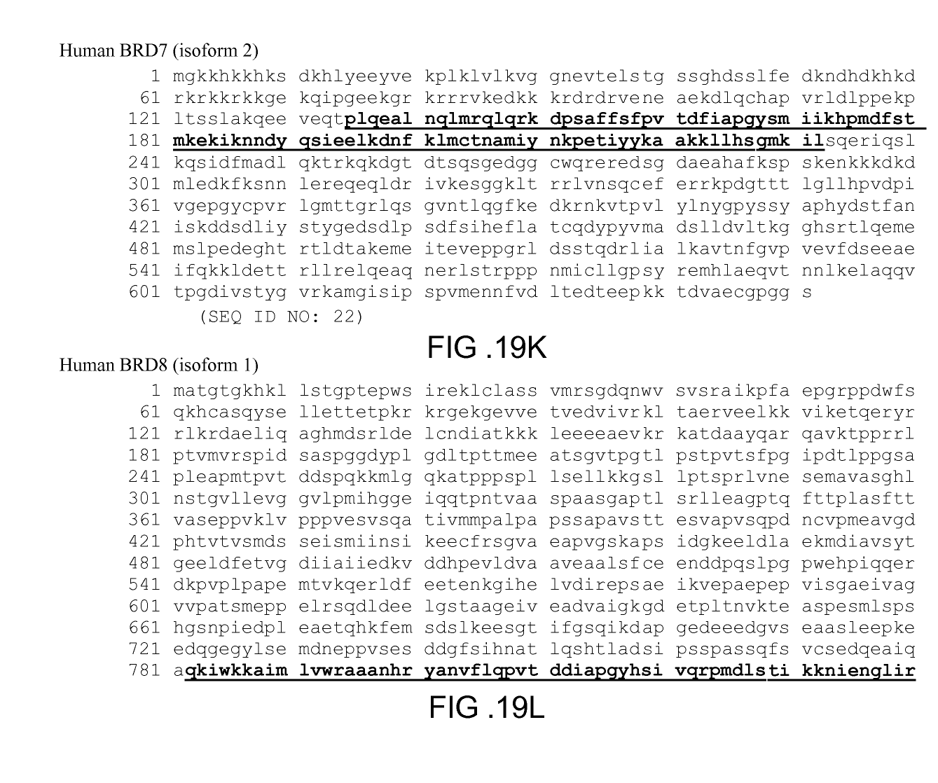

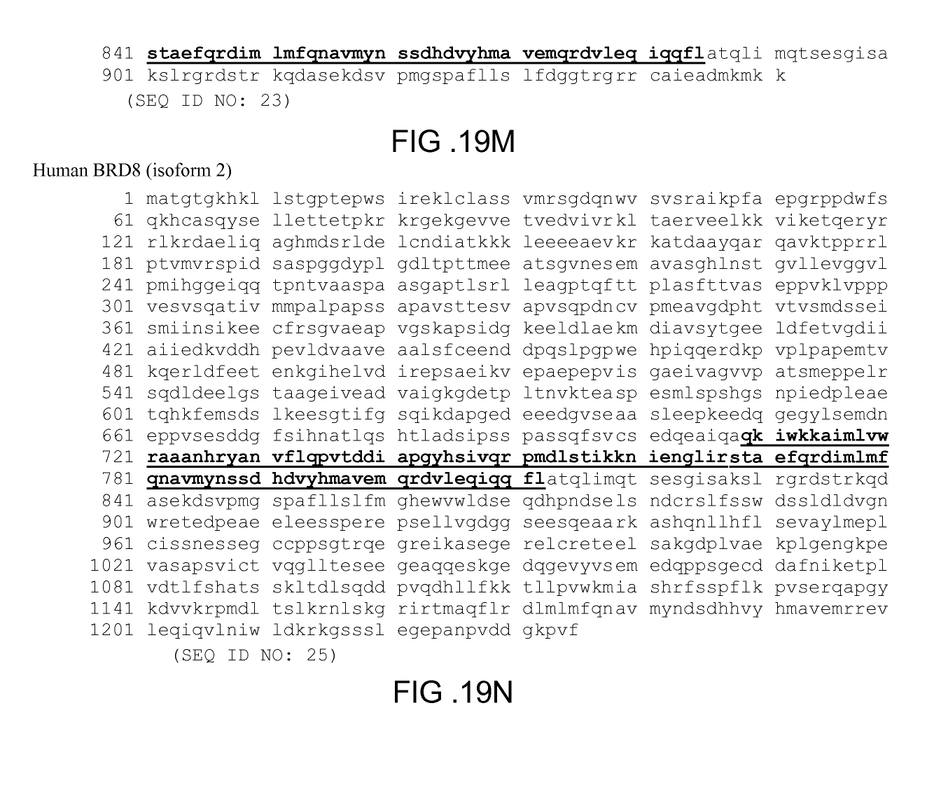

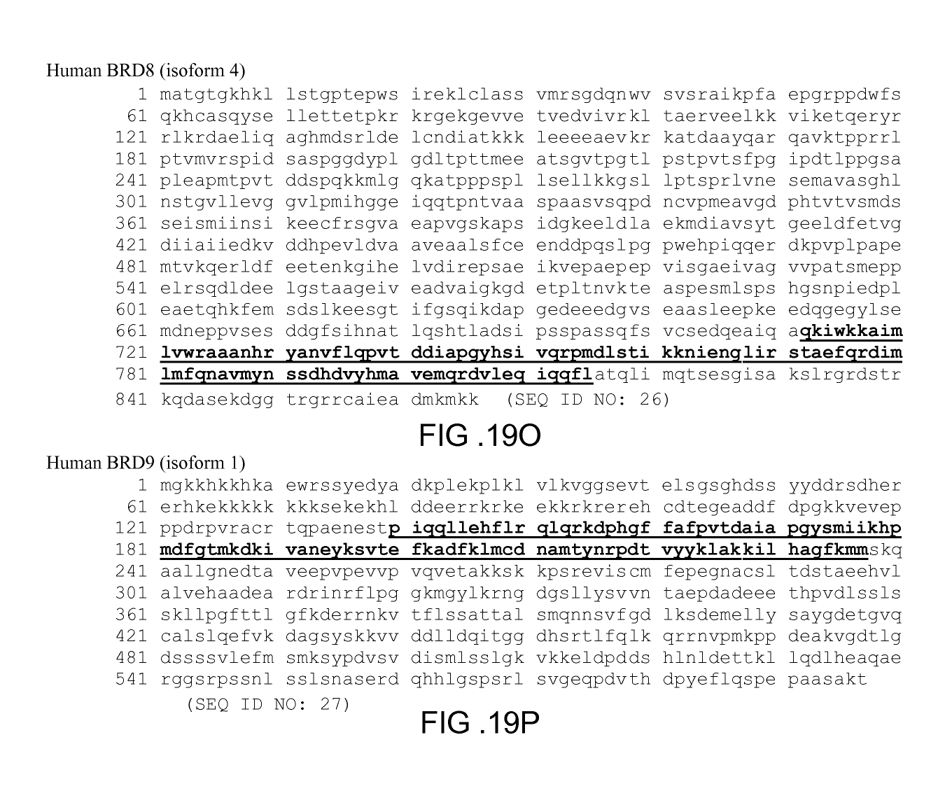

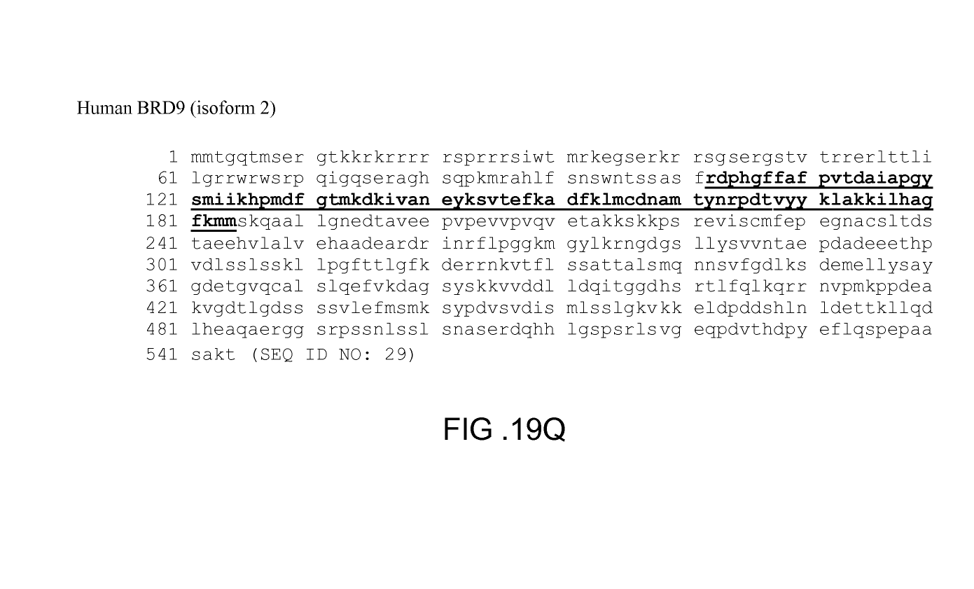

Bromodomains include, but are not limited to, bromodomains present in proteins such as BRD4, PCAF, CBP, BRD1, BRD2, BRD3, BRD6 (BRDT), BRD7, BRD8, and BRD9 (The amino acid sequences of these polypeptides are presented in FIGS. 19A-Q, bromodomains are bolded and underlined). A bromodomain can comprise an amino acid sequence having at least about 75%, at least about 80%, at least about 85%, at least about 90%, at least about 95%, at least about 98%, at least about 99%, or 100%, amino acid sequence identity to a bromodomain depicted in FIGS. 19A-Q.

For example, a bromodomain can comprise an amino acid sequence having at least about 75%, at least about 80%, at least about 85%, at least about 90%, at least about 95%, at least about 98%, at least about 99%, or 100%, amino acid sequence identity to one of the following sequences:

| (SEQ ID NO: 4) | |
|---|---|
| 1) | rqt nqlqyllrvv lktlwkhqfa wpfqqpvdav klnlpdyyki iktpmdmgti kkrlennyyw naqeciqdfn |
| | tmftncyiyn kpgddivlma ealeklflqk inel; |
| (SEQ ID NO: 5) | |
| 2) | lkccsgi lkemfakkha ayawpfykpv dvealglhdy cdiikhpmdm stiksklear eyrdaqefga dvrlmfsncy |
| | kynppdhevv amarklqdvf emrf; |
| (SEQ ID NO: 6) | |
| 3) | peel rqalmptlea lyrqdpeslp frqpvdpqll gipdyfdivk npmdlstikr kldtgqyqep wqyvddvwlm |
| | fnnawlynrk tsrvykfcsk laevfeqeid pvmq; |
| (SEQ ID NO: 7) | |
| 4) | qlys tlksilqqvk shqsawpfme pvkrteapgy yevirfpmdl ktmserlknr yyvskklfma dlqrvftnck |
| | eynaaeseyy kcanilekff fskike; |
| (SEQ ID NO: 10) | |
| 5) | rvtnqlq ylhkvvmkal wkhqfawpfr qpvdavklgl pdyhkiikqp mdmgtikrrl ennyywaase cmqdfntmft |
| | ncyiynkptd divlmaqtle kiflqkvasm; |
| (SEQ ID NO: 11) | |
| 6) | ql khcngilkel lskkhaayaw pfykpvdasa lglhdyhdii khpmdlstvk rkmenrdyrd |
| | aqefaadvrlmfsncykynp pdhdvvamar klqdvfefry; |
| (SEQ ID NO: 13) | |
| 7) | rktnqlq ymqnvvvktl wkhqfawpfy qpvdaiklnl pdyhkiiknp mdmgtikkrl ennyywsase cmqdfntmft |
| | ncyiynkptd divlmaqale kiflqkvaqm; |
| (SEQ ID NO: 14) | |
| 8) | hlrycdsilr emlskkhaay awpfykpvda ealelhdyhd iikhpmdlst vkrkmdgrey pdaqgfaadv |
| | rlmfsncyky nppdhevvam arklqdvfem rf; |
| (SEQ ID NO: 16) | |
| 9) | pltvl lrsvldqlqd kdparifaqp vslkevpdyl dhikhpmdfa tmrkrleaqg yknlhefeed fdliidncmk |
| | ynardtvfyr aavrlrdqgg vvl; |
| (SEQ ID NO: 18) | |
| 10) | rltn qlqylqkvvl kdlwkhsfsw pfqrpvdavk lqlpdyytii knpmdlntik krlenkyyak aseciedfnt |
| | mfsncylynk pgddivlmaq aleklfmqkl sqm; |
| (SEQ ID NO: 19) | |
| 11) | qlrhcseil kemlakkhfs yawpfynpvd vnalglhnyy dvvknpmdlg tikekmdnqe ykdaykfaad |
| | vrlmfmncyk ynppdhevvt marmlqdvfe thf; |
| (SEQ ID NO: 21) | |
| 12) | plqeal nqlmrqlqrk dpsaffsfpv tdfiapgysm iikhpmdfst mkekiknndy qsieelkdnf klmctnamiy |
| | nkpetiyyka akkllhsgmk il; |
| (SEQ ID NO: 24) | |
| 13) | qkiwkkaim lvwraaanhr yanvflqpvt ddiapgyhsi vqrpmdlsti kknienglir staefqrdim lmfqnavmyn |
| | ssdhdvyhma vemqrdvleq iqqfl; |
| and | |
| (SEQ ID NO: 28) | |
| 14) | p iqqllehflr qlqrkdphgf fafpvtdaia pgysmiikhp mdfgtmkdki vaneyksvte fkadfklmcd |
| | namtynrpdt vyyklakkil hagfkmm; |

and can have a length of from about 75 amino acids to about 100 amino acids, or from about 100 amino acids to about 120 amino acids.

An effective amount of an agent that binds a BRD in a cell is an amount that reactivates latent HIV and reduces the reservoir of latent HIV in an individual by at least about 20%, at least about 30%, at least about 40%, at least about 50%, at least about 60%, at least about 70%, at least about 80%, or at least about 90%. A “reduction in the reservoir of latent HIV” (also referred to as “reservoir of latently infected cells”) is a reduction in the number of cells in the individual that harbor a latent HIV infection. Whether the reservoir of latently infected cells is reduced can be determined using any known method, including the method described in Blankson et al. (2000) J. Infect. Disease 182(6):1636-1642.

In some embodiments, a subject method of treating an immunodeficiency virus infection in an individual in need thereof involves: a) administering to the individual an effective amount of a compound that binds a BRD and activates immunodeficiency virus transcription; and b) administering to the individual an effective amount of an agent that inhibits an immunodeficiency virus function. The immunodeficiency virus function can be selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

In some embodiments, a compound that binds a BRD and activates immunodeficiency virus transcription is administered in combination therapy (i.e., co-administered) with: 1) one or more nucleoside reverse transcriptase inhibitors (e.g., Combivir, Epivir, Hivid, Retrovir, Videx, Zerit, Ziagen, etc.); 2) one or more non-nucleoside reverse transcriptase inhibitors (e.g., Rescriptor, Sustiva, Viramune, etc.); 3) one or more protease inhibitors (e.g., Agenerase, Crixivan, Fortovase, Invirase, Kaletra, Norvir, Viracept, etc.); 4) an anti-HIV agent such as a protease inhibitor and a nucleoside reverse transcriptase inhibitor; 5) an anti-HIV agent such as a protease inhibitor, a nucleoside reverse transcriptase inhibitor, and a non-nucleoside reverse transcriptase inhibitor; 6) an anti-HIV agent such as a protease inhibitor and a non-nucleoside reverse transcriptase inhibitor, and/or 7) an anti-viral (e.g., HIV) agent such as a protein kinase C (PKC) activator (e.g., prostratin). Other combinations of an effective amount of a BRD inhibitor with one or more anti-HIV agents, such as one or more of a protease inhibitor, a nucleoside reverse transcriptase inhibitor, a non-nucleoside reverse transcriptase inhibitor, and a protein kinase C (PKC) activator are contemplated.

A PKC activator (e.g., prostratin ((1aR,1bS,4aR,7aS,7bR,8R,9aS)-4a,7b-dihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl)) can be administered in a separate formulation from a BRD inhibitor. A PKC activator can be co-formulated with a BRD inhibitor, and the co-formulation administered to an individual.

In some embodiments, the co-administration of compounds results in synergism, and the combination is therefore a synergistic combination. As used herein, a “synergistic combination” or a “synergistic amount” of (i) a compound that binds a BRD and activates immunodeficiency virus transcription and (ii) an anti-viral agent (e.g., a nucleoside reverse transcriptase inhibitor, a non-nucleoside reverse transcriptase inhibitor, a protease inhibitor, an anti-HIV agent, a protein kinase C (PKC) activator, etc.) is an amount that is more effective in activating immunodeficiency virus transcription when co-administered than the incremental increase that could be predicted or expected from a merely additive combination of (i) and (ii) when each is administered at the same dosage alone (not co-administered).

As one non-limiting example, in some cases, a compound that binds a BRD and activates immunodeficiency virus transcription (e.g., JQ1) is administered in combination therapy with a protein kinase C (PKC) activator (e.g., prostratin, which is a compound that activates PKC and induces HIV expression in latently infected cells, thus antagonizing HIV latency) and this combination (co-administration) is a synergistic combination. As such, the co-administration is more effective in activating immunodeficiency virus transcription than when either compound is administered alone (i.e., not co-administered) and is more effective than would be predicted or expected from merely adding the effects of the compounds when administered alone.

Any of a variety of methods can be used to determine whether a treatment method is effective. For example, methods of determining whether the methods of the invention are effective in reducing immunodeficiency virus (e.g., HIV) viral load, and/or treating an immunodeficiency virus (e.g., HIV) infection, are any known test for indicia of immunodeficiency virus (e.g., HIV) infection, including, but not limited to, measuring viral load, e.g., by measuring the amount of immunodeficiency virus (e.g., HIV) in a biological sample, e.g., using a polymerase chain reaction (PCR) with primers specific for an immunodeficiency virus (e.g., HIV) polynucleotide sequence; detecting and/or measuring a polypeptide encoded by an immunodeficiency virus (e.g., HIV), e.g., p24, gp120, reverse transcriptase, using, e.g., an immunological assay such as an enzyme-linked immunosorbent assay (ELISA) with an antibody specific for the polypeptide; and measuring the CD4⁺ T cell count in the individual.

### Bromodomain Inhibitor Compounds

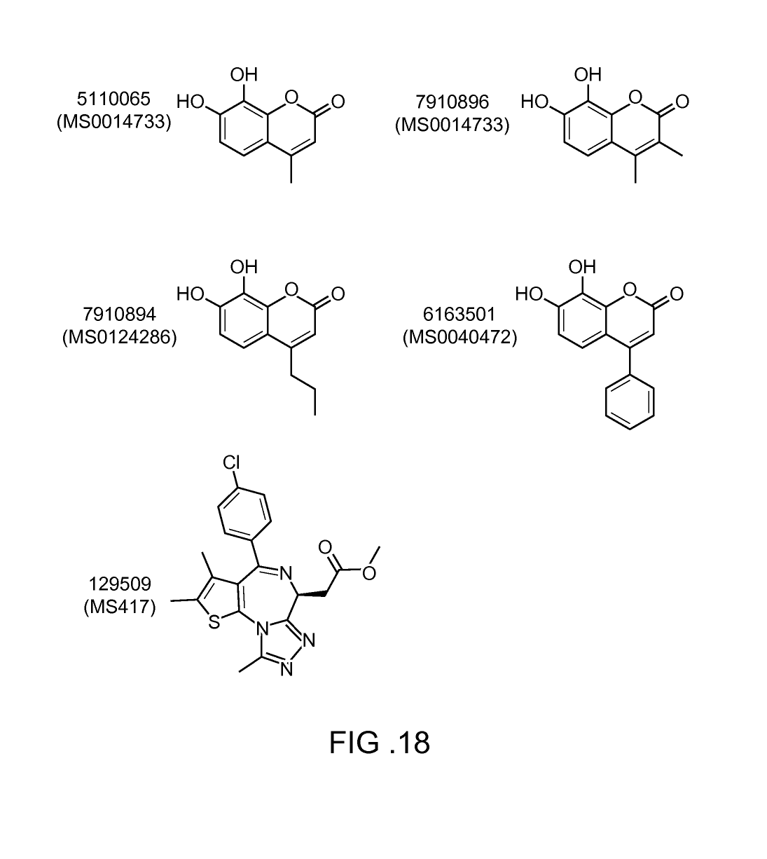

Bromodomain inhibitor compounds that are suitable for use in a subject method include a compound of any of Formulae I-VI, as described below. Exemplary compounds are depicted in FIG. 18. Such compounds are also referred to herein as “BRD inhibitors.”

### Compounds of Formulae I and II

The compositions of the present disclosure include compounds of formulae I and II, shown below. Pharmaceutical compositions and methods of the present disclosure also contemplate compounds of formulae I and II.

In one of its composition aspects, the present embodiments provide a compound of formula I:

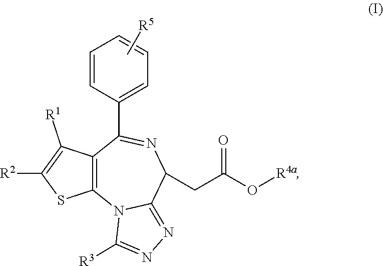

wherein

R¹ is selected from hydrogen, alkyl, substituted alkyl, alkenyl, substituted alkenyl, alkynyl, substituted alkynyl, alkoxy, substituted alkoxy, and acyl;

R² is selected from hydrogen, alkyl, substituted alkyl, alkenyl, substituted alkenyl, alkynyl, substituted alkynyl, alkoxy, substituted alkoxy, and acyl;

R³ is selected from hydrogen, alkyl, substituted alkyl, alkenyl, substituted alkenyl, alkynyl, substituted alkynyl, alkoxy, substituted alkoxy, and acyl;

R^{4a} is selected from hydrogen, C₁-C₃ alkyl, C₅-C₁₀ alkyl, and substituted alkyl;

R⁵ is selected from hydrogen, alkyl, substituted alkyl, alkenyl, substituted alkenyl, alkynyl, substituted alkynyl, hydroxy, alkoxy, substituted alkoxy, acyloxy, thiol, acyl, amino, substituted amino, aminoacyl, acylamino, azido, carboxyl, carboxylalkyl, cyano, halogen, and nitro;

and salts or solvates or stereoisomers thereof.

In formula I, R¹ is selected from hydrogen, alkyl, substituted alkyl, alkenyl, substituted alkenyl, alkynyl, substituted alkynyl, alkoxy, substituted alkoxy, and acyl. In certain instances, R¹ is hydrogen. In certain instances, R¹ is alkyl or substituted alkyl. In certain instances, R¹ is alkyl, such as C₁-C₆ alkyl, including C₁-C₃ alkyl. In certain instances, R¹ is methyl, ethyl, n-propyl, or isopropyl. In certain instances, R¹ is methyl. In certain instances, R¹ is alkenyl or substituted alkenyl. In certain instances, R¹ is selected from alkynyl or substituted alkynyl. In certain instances, R¹ is alkoxy or substituted alkoxy. In certain instances, R¹ is acyl.

In formula I, R² is selected from hydrogen, alkyl, substituted alkyl, alkenyl, substituted alkenyl, alkynyl, substituted alkynyl, alkoxy, substituted alkoxy, and acyl. In certain instances, R² is hydrogen. In certain instances, R² is alkyl or substituted alkyl. In certain instances, R² is alkyl, such as C₁-C₆ alkyl, including C₁-C₃ alkyl. In certain instances, R² is methyl, ethyl, n-propyl, or isopropyl. In certain instances, R² is methyl. In certain instances, R² is alkenyl or substituted alkenyl. In certain instances, R² is selected from alkynyl or substituted alkynyl. In certain instances, R² is alkoxy or substituted alkoxy. In certain instances, R² is acyl.

In formula I, R³ is selected from hydrogen, alkyl, substituted alkyl, alkenyl, substituted alkenyl, alkynyl, substituted alkynyl, alkoxy, substituted alkoxy, and acyl. In certain instances, R³ is hydrogen. In certain instances, R³ is alkyl or substituted alkyl. In certain instances, R³ is alkyl, such as C₁-C₆ alkyl, including C₁-C₃ alkyl. In certain instances, R³ is methyl, ethyl, n-propyl, or isopropyl. In certain instances, R³ is methyl. In certain instances, R³ is alkenyl or substituted alkenyl. In certain instances, R³ is selected from alkynyl or substituted alkynyl. In certain instances, R³ is alkoxy or substituted alkoxy. In certain instances, R³ is acyl.

In formula I, R^{4a} is selected from hydrogen, C₁-C₃ alkyl, C₅-C₁₀ alkyl, and substituted alkyl. In certain instances, R^{4a} is hydrogen. In certain instances, R^{4a} is C₁-C₃ alkyl. In certain instances, R^{4a} is C₅-C₁₀ alkyl. In certain instances, R^{4a} is substituted alkyl. In certain instances, R^{4a} is methyl, ethyl, n-propyl, or isopropyl. In certain instances, R^{4a} is methyl.

In formula I, R⁵ is selected from hydrogen, alkyl, substituted alkyl, alkenyl, substituted alkenyl, alkynyl, substituted alkynyl, hydroxy, alkoxy, substituted alkoxy, acyloxy, thiol, acyl, amino, substituted amino, aminoacyl, acylamino, azido, carboxyl, carboxylalkyl, cyano, halogen, and nitro.

In certain instances, R⁵ is hydrogen. In certain instances, R⁵ is alkyl or substituted alkyl. In certain instances, R⁵ is alkenyl or substituted alkenyl. In certain instances, R⁵ is alkynyl or substituted alkynyl. In certain instances, R⁵ is hydroxy, alkoxy, substituted alkoxy, or acyloxy. In certain instances, R⁵ is thiol. In certain instances, R⁵ is acyl. In certain instances, R⁵ is amino, substituted amino, aminoacyl, acylamino, or azido. In certain instances, R⁵ is carboxyl or carboxylalkyl. In certain instances, R⁵ is cyano. In certain instances, R⁵ is nitro. In certain instances, R⁵ is halogen. In certain instances, R⁵ is fluoro. In certain instances, R⁵ is chloro. In certain instances, R⁵ is bromo.

In certain instances, formula I is the following formula:

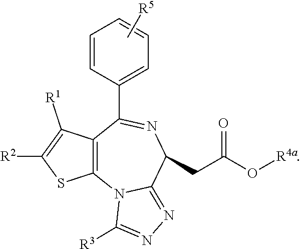

A particular compound of interest, and salts or solvates or stereoisomers thereof, includes:

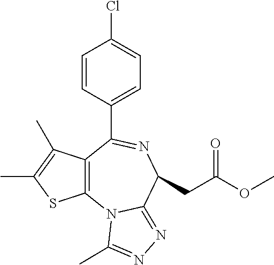

(Methyl 2-((6S)-4-(4-chlorophenyl)-2,3,9-trimethyl-6H-thieno[3,2-f][1,2,4]triazolo[4,3-a][1,4]diazepin-6-yl)acetate).

In one of its composition aspects, the present embodiments provide a compound of formula II:

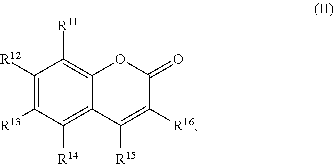

wherein

R¹¹ and R¹² are independently selected from hydroxy, alkoxy, substituted alkoxy, acyloxy, thiol, acyl, amino, substituted amino, aminoacyl, acylamino, azido, carboxyl, carboxylalkyl, cyano, halogen, and nitro;

R¹³ and R¹⁴ are independently selected from hydrogen, alkyl, and substituted alkyl;

R¹⁵ is selected from hydrogen, alkyl, substituted alkyl, aryl, and substituted aryl; and

R¹⁶ is selected from alkyl, substituted alkyl, aryl, and substituted aryl;

and salts or solvates or stereoisomers thereof.

In formula II, R¹¹ and R¹² are independently selected from hydroxy, alkoxy, substituted alkoxy, acyloxy, thiol, acyl, amino, substituted amino, aminoacyl, acylamino, azido, carboxyl, carboxylalkyl, cyano, halogen, and nitro.

In certain instances, R¹¹ is hydroxy. In certain instances, R¹¹ is alkoxy or substituted alkoxy. In certain instances, R¹¹ is acyloxy. In certain instances, R¹¹ is thiol. In certain instances, R¹¹ is acyl. In certain instances, R¹¹ is amino, substituted amino, aminoacyl, acylamino, or azido. In certain instances, R¹¹ is carboxyl or carboxylalkyl. In certain instances, R¹¹ is cyano. In certain instances, R¹¹ is nitro. In certain instances, R¹¹ is halogen. In certain instances, R¹¹ is fluoro. In certain instances, R¹¹ is chloro. In certain instances, R¹¹ is bromo.

In certain instances, R¹² is hydroxy. In certain instances, R¹² is alkoxy or substituted alkoxy. In certain instances, R¹² is acyloxy. In certain instances, R¹² is thiol. In certain instances, R¹² is acyl. In certain instances, R¹² is amino, substituted amino, aminoacyl, acylamino, or azido. In certain instances, R¹² is carboxyl or carboxylalkyl. In certain instances, R¹² is cyano. In certain instances, R¹² is nitro. In certain instances, R¹² is halogen. In certain instances, R¹² is fluoro. In certain instances, R¹² is chloro. In certain instances, R¹² is bromo.

In formula II, R¹³ and R¹⁴ are independently selected from hydrogen, alkyl, and substituted alkyl. In certain instances, R¹³ is hydrogen. In certain instances, R¹³ is alkyl. In certain instances, R¹³ is substituted alkyl. In certain instances, R¹⁴ is hydrogen. In certain instances, R¹⁴ is alkyl. In certain instances, R¹⁴ is substituted alkyl. In certain instances, R¹³ and R¹⁴ are hydrogen.

In formula II, R¹⁵ is selected from hydrogen, alkyl, substituted alkyl, aryl, and substituted aryl. In certain instances, R¹⁵ is hydrogen. In certain instances, R¹⁵ is alkyl or substituted alkyl. In certain instances, R¹⁵ is alkyl, such as C₁-C₆ alkyl, including C₁-C₄ alkyl. In certain instances, R¹⁵ is methyl, ethyl, n-propyl, isopropyl, n-butyl, isobutyl, or tert-butyl. In certain instances, R¹⁵ is methyl. In certain instances, R¹⁵ is aryl or substituted aryl. In certain instances, R¹⁵ is phenyl.

In formula II, R¹⁶ is selected from alkyl, substituted alkyl, aryl, and substituted aryl. In certain instances, R¹⁶ is alkyl or substituted alkyl. In certain instances, R¹⁶ is alkyl, such as C₁-C₆ alkyl, including C₁-C₄ alkyl. In certain instances, R¹⁶ is methyl, ethyl, n-propyl, isopropyl, n-butyl, isobutyl, or tert-butyl. In certain instances, R¹⁶ is methyl. In certain instances, R¹⁶ is aryl or substituted aryl. In certain instances, R¹⁶ is phenyl.

A particular compound of interest, and salts or solvates or stereoisomers thereof, includes:

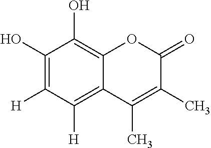

(7,8-dihydroxy-3,4-dimethyl-2H-chromen-2-one).

As discussed above, the present disclosure provides methods of treating an immunodeficiency virus infection in an individual, the methods generally involving co-administering to the individual an agent, such as a compound of Formulae (I) or (II), that reactivates latent HIV and an anti-HIV agent. The present disclosure provides methods for reducing the reservoir of latent immunodeficiency virus in an individual by administering to the individual an effective amount of an agent, such as a compound of Formulae (I) or (II), that binds a BRD in a cell latently infected with HIV.

### Compounds of Formulas III-VI

The present disclosure also provides methods of treating an immunodeficiency virus infection in an individual, the methods generally involving co-administering to the individual an agent, such as a compound of Formulas III-VI, that reactivates latent HIV and an anti-HIV agent. The present disclosure provides methods for reducing the reservoir of latent immunodeficiency virus in an individual by administering to the individual an effective amount of an agent, such as a compound of Formulae III-VI, that binds a BRD in a cell latently infected with HIV.

The disclosure provides a compound of formula III for use in the methods of the embodiments:

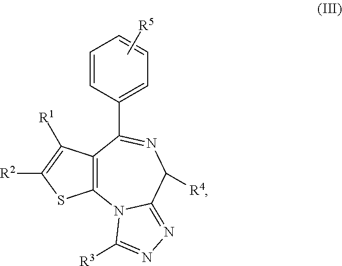

wherein

R¹ is selected from hydrogen, alkyl, substituted alkyl, alkenyl, substituted alkenyl, alkynyl, substituted alkynyl, alkoxy, substituted alkoxy, and acyl;

R² is selected from hydrogen, alkyl, substituted alkyl, alkenyl, substituted alkenyl, alkynyl, substituted alkynyl, alkoxy, substituted alkoxy, and acyl;

R³ is selected from hydrogen, alkyl, substituted alkyl, alkenyl, substituted alkenyl, alkynyl, substituted alkynyl, alkoxy, substituted alkoxy, and acyl;

R⁴ is selected from hydrogen, alkyl, substituted alkyl, alkenyl, substituted alkenyl, alkynyl, substituted alkynyl, alkoxy, substituted alkoxy, acyl, alkoxycarbonyl, and aminoacyl;

R⁵ is selected from hydrogen, alkyl, substituted alkyl, alkenyl, substituted alkenyl, alkynyl, substituted alkynyl, hydroxy, alkoxy, substituted alkoxy, acyloxy, thiol, acyl, amino, substituted amino, aminoacyl, acylamino, azido, carboxyl, carboxylalkyl, cyano, halogen, and nitro;

and salts or solvates or stereoisomers thereof.

In formula III, R¹ is selected from hydrogen, alkyl, substituted alkyl, alkenyl, substituted alkenyl, alkynyl, substituted alkynyl, alkoxy, substituted alkoxy, and acyl. In certain instances, R¹ is hydrogen. In certain instances, R¹ is alkyl or substituted alkyl. In certain instances, R¹ is alkyl, such as C₁-C₆ alkyl, including C₁-C₃ alkyl. In certain instances, R¹ is methyl, ethyl, n-propyl, or isopropyl. In certain instances, R¹ is methyl. In certain instances, R¹ is alkenyl or substituted alkenyl. In certain instances, R¹ is selected from alkynyl or substituted alkynyl. In certain instances, R¹ is alkoxy or substituted alkoxy. In certain instances, R¹ is acyl.

In formula III, R² is selected from hydrogen, alkyl, substituted alkyl, alkenyl, substituted alkenyl, alkynyl, substituted alkynyl, alkoxy, substituted alkoxy, and acyl. In certain instances, R² is hydrogen. In certain instances, R² is alkyl or substituted alkyl. In certain instances, R² is alkyl, such as C₁-C₆ alkyl, including C₁-C₃ alkyl. In certain instances, R² is methyl, ethyl, n-propyl, or isopropyl. In certain instances, R² is methyl. In certain instances, R² is alkenyl or substituted alkenyl. In certain instances, R² is selected from alkynyl or substituted alkynyl. In certain instances, R² is alkoxy or substituted alkoxy. In certain instances, R² is acyl.

In formula III, R³ is selected from hydrogen, alkyl, substituted alkyl, alkenyl, substituted alkenyl, alkynyl, substituted alkynyl, alkoxy, substituted alkoxy, and acyl. In certain instances, R³ is hydrogen. In certain instances, R³ is alkyl or substituted alkyl. In certain instances, R³ is alkyl, such as C₁-C₆ alkyl, including C₁-C₃ alkyl. In certain instances, R³ is methyl, ethyl, n-propyl, or isopropyl. In certain instances, R³ is methyl. In certain instances, R³ is alkenyl or substituted alkenyl. In certain instances, R³ is selected from alkynyl or substituted alkynyl. In certain instances, R³ is alkoxy or substituted alkoxy. In certain instances, R³ is acyl.

In formula III, R⁴ is selected from hydrogen, alkyl, substituted alkyl, alkenyl, substituted alkenyl, alkynyl, substituted alkynyl, alkoxy, substituted alkoxy, acyl, alkoxycarbonyl, and aminoacyl.

In certain instances, R⁴ is hydrogen. In certain instances, R⁴ is alkyl or substituted alkyl. In certain instances, R⁴ is alkenyl or substituted alkenyl. In certain instances, R⁴ is alkynyl or substituted alkynyl. In certain instances, R⁴ is alkoxy or substituted alkoxy. In certain instances, R⁴ is acyl. In certain instances, R⁴ is alkoxycarbonyl. In certain instances, R⁴ is aminoacyl.

In certain instances, R⁴ is a substituted alkyl, such as a C₁-C₆ substituted alkyl, including a C₁-C₃ substituted alkyl, such as methyl, ethyl, n-propyl, or isopropyl. In certain instances, R⁴ is a substituted methyl. In certain instances, R⁴ is substituted alkyl, which is substituted with acyl, alkoxycarbonyl, or aminoacyl. In certain instances, R⁴ is substituted alkyl, which is substituted with acyl. In certain instances, R⁴ is substituted alkyl, which is substituted with alkoxycarbonyl, such as a C₁-C₆ alkoxycarbonyl or a C₁-C₃ alkoxycarbonyl. In certain instances, R⁴ is substituted alkyl, which is substituted with methoxycarbonyl, In certain instances, R⁴ is substituted alkyl, which is substituted with aminoacyl.

In formula III, R⁵ is selected from hydrogen, alkyl, substituted alkyl, alkenyl, substituted alkenyl, alkynyl, substituted alkynyl, hydroxy, alkoxy, substituted alkoxy, acyloxy, thiol, acyl, amino, substituted amino, aminoacyl, acylamino, azido, carboxyl, carboxylalkyl, cyano, halogen, and nitro.

In certain instances, R⁵ is hydrogen. In certain instances, R⁵ is alkyl or substituted alkyl. In certain instances, R⁵ is alkenyl or substituted alkenyl. In certain instances, R⁵ is alkynyl or substituted alkynyl. In certain instances, R⁵ is hydroxy, alkoxy, substituted alkoxy, or acyloxy. In certain instances, R⁵ is thiol. In certain instances, R⁵ is acyl. In certain instances, R⁵ is amino, substituted amino, aminoacyl, acylamino, or azido. In certain instances, R⁵ is carboxyl or carboxylalkyl. In certain instances, R⁵ is cyano. In certain instances, R⁵ is nitro. In certain instances, R⁵ is halogen. In certain instances, R⁵ is fluoro. In certain instances, R⁵ is chloro. In certain instances, R⁵ is bromo.

In certain instances, formula III is the following formula:

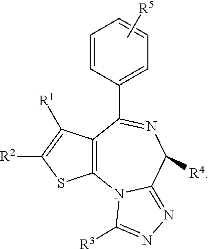

In one of its composition aspects, the present embodiments provide a compound of formula IV:

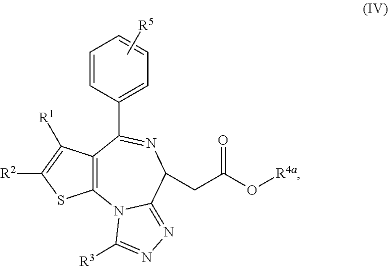

wherein

R¹ is selected from hydrogen, alkyl, substituted alkyl, alkenyl, substituted alkenyl, alkynyl, substituted alkynyl, alkoxy, substituted alkoxy, and acyl;

R² is selected from hydrogen, alkyl, substituted alkyl, alkenyl, substituted alkenyl, alkynyl, substituted alkynyl, alkoxy, substituted alkoxy, and acyl;

R³ is selected from hydrogen, alkyl, substituted alkyl, alkenyl, substituted alkenyl, alkynyl, substituted alkynyl, alkoxy, substituted alkoxy, and acyl;

R^{4a} is selected from hydrogen, alkyl, and substituted alkyl;

R⁵ is selected from hydrogen, alkyl, substituted alkyl, alkenyl, substituted alkenyl, alkynyl, substituted alkynyl, hydroxy, alkoxy, substituted alkoxy, acyloxy, thiol, acyl, amino, substituted amino, aminoacyl, acylamino, azido, carboxyl, carboxylalkyl, cyano, halogen, and nitro; and salts or solvates or stereoisomers thereof.

In formula IV, R¹ is selected from hydrogen, alkyl, substituted alkyl, alkenyl, substituted alkenyl, alkynyl, substituted alkynyl, alkoxy, substituted alkoxy, and acyl. In certain instances, R¹ is hydrogen. In certain instances, R¹ is alkyl or substituted alkyl. In certain instances, R¹ is alkyl, such as C₁-C₆ alkyl, including C₁-C₃ alkyl. In certain instances, R¹ is methyl, ethyl, n-propyl, or isopropyl. In certain instances, R¹ is methyl. In certain instances, R¹ is alkenyl or substituted alkenyl. In certain instances, R¹ is selected from alkynyl or substituted alkynyl. In certain instances, R¹ is alkoxy or substituted alkoxy. In certain instances, R¹ is acyl.

In formula IV, R² is selected from hydrogen, alkyl, substituted alkyl, alkenyl, substituted alkenyl, alkynyl, substituted alkynyl, alkoxy, substituted alkoxy, and acyl. In certain instances, R² is hydrogen. In certain instances, R² is alkyl or substituted alkyl. In certain instances, R² is alkyl, such as C₁-C₆ alkyl, including C₁-C₃ alkyl. In certain instances, R² is methyl, ethyl, n-propyl, or isopropyl. In certain instances, R² is methyl. In certain instances, R² is alkenyl or substituted alkenyl. In certain instances, R² is selected from alkynyl or substituted alkynyl. In certain instances, R² is alkoxy or substituted alkoxy. In certain instances, R² is acyl.

In formula IV, R³ is selected from hydrogen, alkyl, substituted alkyl, alkenyl, substituted alkenyl, alkynyl, substituted alkynyl, alkoxy, substituted alkoxy, and acyl. In certain instances, R³ is hydrogen. In certain instances, R³ is alkyl or substituted alkyl. In certain instances, R³ is alkyl, such as C₁-C₆ alkyl, including C₁-C₃ alkyl. In certain instances, R³ is methyl, ethyl, n-propyl, or isopropyl. In certain instances, R³ is methyl. In certain instances, R³ is alkenyl or substituted alkenyl. In certain instances, R³ is selected from alkynyl or substituted alkynyl. In certain instances, R³ is alkoxy or substituted alkoxy. In certain instances, R³ is acyl.

In formula IV, R^{4a} is selected from hydrogen, alkyl, and substituted alkyl. In certain instances, R^{4a} is hydrogen. In certain instances, R^{4a} is alkyl, such as C₁-C₆ alkyl, including C₁-C₄ alkyl. In certain instances, R^{4a} is substituted alkyl. In certain instances, R^{4a} is methyl, ethyl, n-propyl, isopropyl, n-butyl, isobutyl, or tert-butyl. In certain instances, R^{4a} is methyl. In certain instances, R^{4a} is tert-butyl.

In formula IV, R⁵ is selected from hydrogen, alkyl, substituted alkyl, alkenyl, substituted alkenyl, alkynyl, substituted alkynyl, hydroxy, alkoxy, substituted alkoxy, acyloxy, thiol, acyl, amino, substituted amino, aminoacyl, acylamino, azido, carboxyl, carboxylalkyl, cyano, halogen, and nitro.

In certain instances, R⁵ is hydrogen. In certain instances, R⁵ is alkyl or substituted alkyl. In certain instances, R⁵ is alkenyl or substituted alkenyl. In certain instances, R⁵ is alkynyl or substituted alkynyl. In certain instances, R⁵ is hydroxy, alkoxy, substituted alkoxy, or acyloxy. In certain instances, R⁵ is thiol. In certain instances, R⁵ is acyl. In certain instances, R⁵ is amino, substituted amino, aminoacyl, acylamino, or azido. In certain instances, R⁵ is carboxyl or carboxylalkyl. In certain instances, R⁵ is cyano. In certain instances, R⁵ is nitro. In certain instances, R⁵ is halogen. In certain instances, R⁵ is fluoro. In certain instances, R⁵ is chloro. In certain instances, R⁵ is bromo.

In certain instances, formula IV is the following formula:

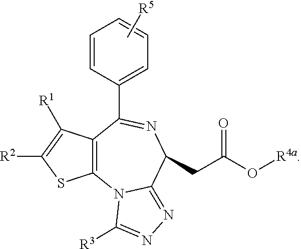

Particular compounds of interest, and salts or solvates or stereoisomers thereof, include:

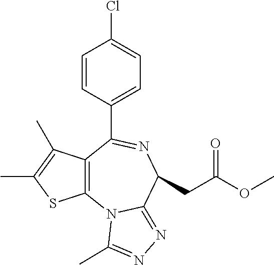

(Methyl 2-((6S)-4-(4-chlorophenyl)-2,3,9-trimethyl-6H-thieno[3,2-f][1,2,4]triazolo[4,3-a][1,4]diazepin-6-yl)acetate); and

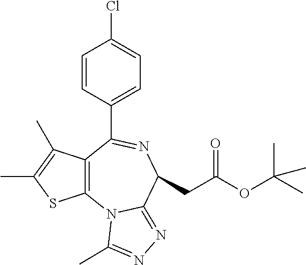 [SMILES: Cc1sc2c(c1C)C(c1ccc(Cl)cc1)=N[C@@H](CC(=O)OC(C)(C)C)c1nnc(C)n1-2]

(tert-Butyl 2-((6S)-4-(4-chlorophenyl)-2,3,9-trimethyl-6H-thieno[3,2-f][1,2,4]triazolo[4,3-a][1,4]diazepin-6-yl)acetate).

The disclosure provides a compound of formula V for use in the methods of the embodiments:

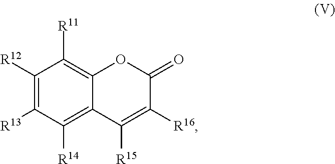

wherein

R¹¹, R¹², R¹³, R¹⁴, R¹⁵, and R¹⁶ are independently selected from the group consisting of hydrogen, hydroxy, lower alkyl, substituted alkyl, aryl, aralkyl, substituted aralkyl, heteroaryl, substituted heteroaryl, sulfonyl, amino, substituted amino, ammonium salt, nitro, alkoxy, substituted alkoxy, carbonate, thiol, halogen, and carboxy;

and salts or solvates or stereoisomers thereof.

In certain instances, R¹¹, R¹², R¹³, R¹⁴, R¹⁵, and R¹⁶ are independently selected from the group consisting of hydrogen, hydroxy, lower alkyl, aryl, phenyl, aralkyl; substituted aralkyl, heteroaryl, substituted heteroaryl, SO₂, NH₂, NH₃⁺, NO₂, SO₂, CH₃, CH₂CH₃, OCH₃, OCOCH₃, CH₂COCH₃, OCH₂CH₃, OCH(CH₃)₂, OCH₂COOH, OCHCH₃COOH, OCH₂COCH₃, OCH₂CONH₂, OCOCH(CH₃)₂, OCH₂CH₂OH, OCH₂CH₂CH₃, O(CH₂)₃CH₃, OCHCH₃COOCH₃, OCH₂CON(CH₃)₂, NH(CH₂)₃N(CH₃)₂, NH(CH₂)₂N(CH₃)₂, NH(CH₂)₂OH, NH(CH₂)₃CH₃, NHCH₃, SH, halogen, carboxy, and alkoxy.

In certain instances, R¹¹, R¹², R¹³, R¹⁴, R¹⁵, and R¹⁶ are independently selected from the group consisting of hydrogen, hydroxy, alkoxy, halogen, lower alkyl, and aryl.

In certain instances, R¹¹ and R¹² are independently selected from the group consisting of hydrogen, hydroxyl, alkoxy, and halogen. In certain instances, R¹¹ and R¹² are independently selected from the group consisting of hydrogen and hydroxy. In certain instances, R¹¹ and R¹² are hydroxy.

In certain instances, R¹³ and R¹⁴ are independently selected from the group consisting of hydrogen, lower alkyl, and aryl. In certain instances, R¹³ and R¹⁴ are hydrogen. In certain instances, R¹³ is lower alkyl. In certain instances, R¹⁴ is lower alkyl, such as C₁-C₆ alkyl, including C₁-C₄ alkyl. In certain instances, R¹³ is aryl. In certain instances, R¹⁴ is aryl.

In certain instances, R¹⁵ is selected from the group consisting of hydrogen, lower alkyl, and aryl. In certain instances, R¹⁵ is hydrogen. In certain instances, R¹⁵ is lower alkyl, such as C₁-C₆ alkyl, including C₁-C₄ alkyl. In certain instances, R¹⁵ is methyl, ethyl, n-propyl, isopropyl, n-butyl, isobutyl, or tert-butyl. In certain instances, R¹⁵ is methyl. In certain instances, R¹⁵ is n-propyl. In certain instances, R¹⁵ is aryl. In certain instances, R¹⁵ is phenyl.

In certain instances, R¹⁶ is selected from the group consisting of hydrogen, lower alkyl, and aryl. In certain instances, R¹⁶ is hydrogen. In certain instances, R¹⁶ is lower alkyl, such as C₁-C₆ alkyl, including C₁-C₄ alkyl. In certain instances, R¹⁶ is methyl, ethyl, n-propyl, isopropyl, n-butyl, isobutyl, or tert-butyl. In certain instances, R¹⁶ is methyl. In certain instances, R¹⁶ is n-propyl. In certain instances, R¹⁶ is aryl. In certain instances, R¹⁶ is phenyl.

The disclosure provides a compound of formula VI for use in the methods of the embodiments:

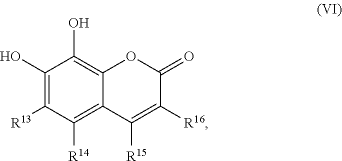

wherein

R¹³, R¹⁴, R¹⁵, and R¹⁶ are independently selected from the group consisting of hydrogen, hydroxy, lower alkyl, substituted alkyl, aryl, aralkyl, substituted aralkyl, heteroaryl, substituted heteroaryl, sulfonyl, amino, substituted amino, ammonium salt, nitro, alkoxy, substituted alkoxy, carbonate, thiol, halogen, and carboxy;

and salts or solvates or stereoisomers thereof.

In certain instances, R¹³, R¹⁴, R¹⁵, and R¹⁶ are independently selected from the group consisting of hydrogen, hydroxy, lower alkyl, aryl, phenyl, aralkyl; substituted aralkyl, heteroaryl, substituted heteroaryl, SO₂, NH₂, NH₃⁺, NO₂, SO₂, CH₃, CH₂CH₃, OCH₃, OCOCH₃, CH₂COCH₃, OCH₂CH₃, OCH(CH₃)₂, OCH₂COOH, OCHCH₃COOH, OCH₂COCH₃, OCH₂CONH₂, OCOCH(CH₃)₂, OCH₂CH₂OH, OCH₂CH₂CH₃, O(CH₂)₃CH₃, OCHCH₃COOCH₃, OCH₂CON(CH₃)₂, NH(CH₂)₃N(CH₃)₂, NH(CH₂)₂N(CH₃)₂, NH(CH₂)₂OH, NH(CH₂)₃CH₃, NHCH₃, SH, halogen, carboxy, and alkoxy.

In certain instances, R¹³, R¹⁴, R¹⁵, and R¹⁶ are independently selected from the group consisting of hydrogen, hydroxy, alkoxy, halogen, lower alkyl, and aryl.

In certain instances, R¹³ and R¹⁴ are independently selected from the group consisting of hydrogen, lower alkyl, and aryl. In certain instances, R¹³ and R¹⁴ are hydrogen. In certain instances, R¹³ is lower alkyl. In certain instances, R¹⁴ is lower alkyl, such as C₁-C₆ alkyl, including C₁-C₄ alkyl. In certain instances, R¹³ is aryl. In certain instances, R¹⁴ is aryl.

In certain instances, R¹⁵ is selected from the group consisting of hydrogen, lower alkyl, and aryl. In certain instances, R¹⁵ is hydrogen. In certain instances, R¹⁵ is lower alkyl, such as C₁-C₆ alkyl, including C₁-C₄ alkyl. In certain instances, R¹⁵ is methyl, ethyl, n-propyl, isopropyl, n-butyl, isobutyl, or tert-butyl. In certain instances, R¹⁵ is methyl. In certain instances, R¹⁵ is n-propyl. In certain instances, R¹⁵ is aryl. In certain instances, R¹⁵ is phenyl.

In certain instances, R¹⁶ is selected from the group consisting of hydrogen, lower alkyl, and aryl. In certain instances, R¹⁶ is hydrogen. In certain instances, R¹⁶ is lower alkyl, such as C₁-C₆ alkyl, including C₁-C₄ alkyl. In certain instances, R¹⁶ is methyl, ethyl, n-propyl, isopropyl, n-butyl, isobutyl, or tert-butyl. In certain instances, R¹⁶ is methyl. In certain instances, R¹⁶ is n-propyl. In certain instances, R¹⁶ is aryl. In certain instances, R¹⁶ is phenyl.

Particular compounds of interest, and salts or solvates or stereoisomers thereof, include:

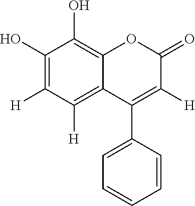 [SMILES: O=c1cc(-c2ccccc2)c2ccc(O)c(O)c2o1]

(7,8-dihydroxy-4-phenyl-2H-chromen-2-one);

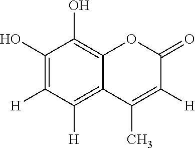

(7,8-dihydroxy-4-methyl-2H-chromen-2-one);

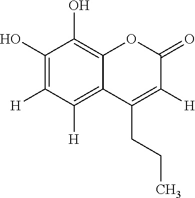 [SMILES: CCCc1cc(=O)oc2c(O)c(O)ccc12]

(7,8-dihydroxy-4-propyl-2H-chromen-2-one); and

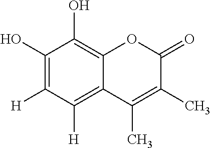

(7,8-dihydroxy-3,4-dimethyl-2H-chromen-2-one).

Compounds of Formulae VII-XIV

Aspects of the present disclosure further include compounds of formulae VII-XIV, shown below. Pharmaceutical compositions and methods of the present disclosure also contemplate compounds of formulae VII-XIV.

In one of its composition aspects, the present embodiments provide a compound of formula VII:

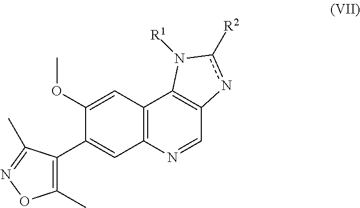

wherein

R¹ is selected from alkyl, substituted alkyl, alkenyl, substituted alkenyl, alkynyl, substituted alkynyl, cycloalkyl, substituted cycloalkyl, heterocycyl, substituted heterocycyl, aryl, substituted aryl, heteroaryl, and substituted heteroaryl;

R² is selected from alkyl, substituted alkyl, alkenyl, substituted alkenyl, alkynyl, substituted alkynyl, cycloalkyl, substituted cycloalkyl, heterocycyl, substituted heterocycyl, aryl, substituted aryl, heteroaryl, substituted heteroaryl, and oxo;

and salts or solvates or stereoisomers thereof.

In certain embodiments, R¹ is selected from alkyl, substituted alkyl, alkenyl, substituted alkenyl, alkynyl, substituted alkynyl, cycloalkyl, substituted cycloalkyl, heterocycyl, substituted heterocycyl, aryl, substituted aryl, heteroaryl, and substituted heteroaryl.

In certain instances, R¹ is alkyl, such as a C₁-C₆ alkyl, including a C₁-C₄ alkyl, or a C₁-C₃ alkyl. For example, R¹ may be a methyl, ethyl or propyl. In certain instances, R¹ is a substituted alkyl, such as a C₁-C₆ substituted alkyl, including a C₁-C₄ substituted alkyl, or a C₁-C₃ substituted alkyl. In embodiments where R¹ is a substituted alkyl, the substituents on the alkyl group may include one or more of alkyl, alkenyl, alkynyl, cycloalkyl, substituted cycloalkyl, heterocycyl, substituted heterocycyl, aryl, substituted aryl, heteroaryl, substituted heteroaryl, hydroxyl, amino, alkoxy, halogen, and combinations thereof. In certain instances, R¹ is a substituted alkyl, where the alkyl is substituted with a cycloalkyl. In certain instances, R¹ is a substituted alkyl, where the alkyl is substituted with a substituted cycloalkyl. In certain instances, R¹ is a substituted alkyl, where the alkyl is substituted with a heterocycyl. In certain instances, R¹ is a substituted alkyl, where the alkyl is substituted with a substituted heterocycyl. In certain instances, R¹ is a substituted alkyl, where the alkyl is substituted with an aryl. In certain instances, R¹ is a substituted alkyl, where the alkyl is substituted with a substituted aryl. In certain instances, R¹ is a substituted alkyl, where the alkyl is substituted with a heteroaryl, such as a heteroaryl where one or more ring atoms is an N, O or S (e.g., pyridine, pyrimidine, and the like). In certain instances, R¹ is a substituted alkyl, where the alkyl is substituted with a substituted heteroaryl. In certain instances, R¹ is a substituted alkyl, where the alkyl is substituted with a hydroxyl. In certain instances, R¹ is a substituted alkyl, where the alkyl is substituted with an amino. In certain instances, R¹ is a substituted alkyl, where the alkyl is substituted with an alkoxy, such as a C₁-C₆ alkoxy, including a C₁-C₄ alkoxy, or a C₁-C₃ alkoxy (e.g., methoxy, ethoxy or propoxy).

In certain instances, R¹ is alkenyl. In certain instances, R¹ is substituted alkenyl. In certain instances, R¹ is alkynyl. In certain instances, R¹ is substituted alkynyl. In certain instances, R¹ is cycloalkyl. In certain instances, R¹ is substituted cycloalkyl. In certain instances, R¹ is heterocycyl. In certain instances, R¹ is substituted heterocycyl. In certain instances, R¹ is aryl. In certain instances, R¹ is substituted aryl. In certain instances, R¹ is heteroaryl. In certain instances, R¹ is substituted heteroaryl.

In certain embodiments, R² is selected from alkyl, substituted alkyl, alkenyl, substituted alkenyl, alkynyl, substituted alkynyl, cycloalkyl, substituted cycloalkyl, heterocycyl, substituted heterocycyl, aryl, substituted aryl, heteroaryl, substituted heteroaryl, and oxo.

In certain instances, R² is alkyl, such as a C₁-C₆ alkyl, or a C₁-C₄ alkyl, or a C₁-C₃ alkyl. In certain instances, R² is substituted alkyl, such as a C₁-C₆ substituted alkyl, or a C₁-C₄ substituted alkyl, or a C₁-C₃ substituted alkyl. In certain embodiments, R² is alkenyl, such as a C₁-C₆ alkenyl, or a C₁-C₄ alkenyl, or a C₁-C₃ alkenyl. In certain embodiments, R² is substituted alkenyl, such as a C₁-C₆ substituted alkenyl, or a C₁-C₄ substituted alkenyl, or a C₁-C₃ substituted alkenyl. In certain embodiments, R² is alkynyl, such as a C₁-C₆ alkynyl, or a C₁-C₄ alkynyl, or a C₁-C₃ alkynyl. In certain embodiments, R² is substituted alkynyl, such as a C₁-C₆ substituted alkynyl, or a C₁-C₄ substituted alkynyl, or a C₁-C₃ substituted alkynyl.

In certain embodiments, R² is cycloalkyl or substituted cycloalkyl. In certain embodiments, R² is cycloalkyl, such as a C₃-C₇ cycloalkyl. In certain embodiments, R² is substituted cycloalkyl, such as a C₃-C₇ substituted cycloalkyl. In certain embodiments, R² is heterocycyl or substituted heterocycyl. In certain embodiments, R² is heterocycyl, such as a C₃-C₇ heterocycyl. In some instances, R² is a heterocycyl, where one or more ring atoms is N, O or S. For example, R² may be a heterocycyl where one or more ring atoms is O, such as, but not limited to, oxirane, oxetane, tetrahydrofuran, tetrahydropyran, and the like. In some instances, R² is tetrahydropyran. In certain embodiments, R² is substituted heterocycyl, such as a C₃-C₇ substituted heterocycyl.

In certain embodiments, R² is aryl or substituted aryl. In certain embodiments, R² is aryl. In certain embodiments, R² is substituted aryl. In certain embodiments, R² is heteroaryl or substituted heteroaryl. In certain embodiments, R² is heteroaryl. In certain embodiments, R² is substituted heteroaryl.

In certain embodiments, R² is oxo. In embodiments were R² is oxo, the dashed line in formula VII is not a bond.

Particular compounds of interest, and salts or solvates or stereoisomers thereof, include:

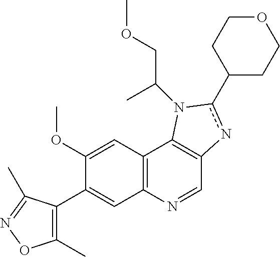

(4-(8-methoxy-1-(1-methoxypropan-2-yl)-2-(tetrahydro-2H-pyran-4-yl)-1H-imidazo[4,5-c]quinolin-7-yl)-3,5-dimethylisoxazole); and

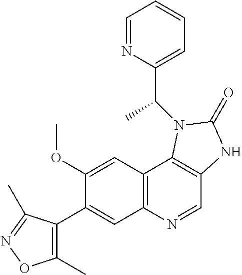 [SMILES: COc1cc2c(cc1-c1c(C)noc1C)ncc1[nH]c(=O)n([C@H](C)c3ccccn3)c12]

(7-(3,5-dimethylisoxazol-4-yl)-8-methoxy-1-((R)-1-(pyridin-2-yl)ethyl)-1H-imidazo[4,5-c]quinolin-2(3H)-one).

In one of its composition aspects, the present embodiments provide a compound of formula VIII:

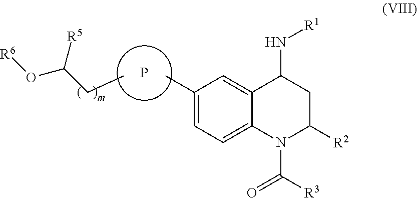

wherein

P is pyrazolyl or triazolyl;

R¹ is —C(O)OR⁴ in which R⁴ is C₁₋₃ alkyl or C₃₋₇ cycloalkyl; or

R¹ is a group selected from phenyl, pyridyl, pyrazinyl and pyrimidinyl, said groups being optionally substituted by one or two substituents selected from halogen, C₁₋₄ alkyl and CN;

R² is C₁₋₄ alkyl;

R³ is C₁₋₄ alkyl;

R⁵ is H or C₁₋₄ alkyl;

R⁶ is C₁₋₄ alkyl;

or R⁵ and R⁶, together with the O to which R⁶ is attached, form an oxetanyl, tetrahydrofuranyl or tetrahydropyranyl ring; and

m is 1 or 2;

and salts or solvates or stereoisomers thereof.

In certain embodiments, compounds of formula VIII include compounds with cis relative stereochemistry across the tetrahydroquinoline ring with respect to the substituents in the 2 and 4 positions on the ring. In some embodiments, the compound of formula VIII or a salt thereof is the (2S, 4R) enantiomer.

In certain embodiments, P is

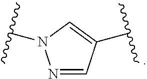 [SMILES: *c1cnn(*)c1]

In certain embodiments, P is selected from:

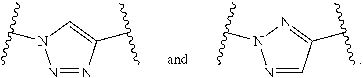 [SMILES: *c1cn(*)nn1 ; *c1cnn(*)n1]

In certain embodiments, R¹ is —C(O)OR⁴ in which R⁴ is isopropyl.

In certain embodiments, R¹ is selected from:

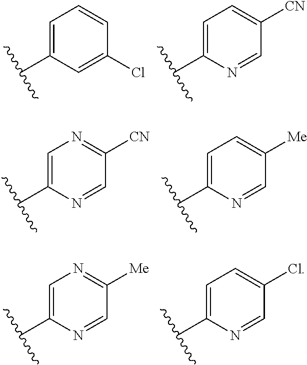

In certain embodiments, R² is methyl.

In certain embodiments, R³ is methyl.

In certain embodiments, m is 1.

In certain embodiments, R⁵ is hydrogen.

In certain embodiments, R⁶ is methyl.

As used herein, the term “substituted” refers to substitution with the named substituent or substituents, multiple degrees of substitution being allowed unless otherwise stated. When the substituent is on a ring comprising a heteroatom the substituent may be located on a carbon or a heteroatom, if the latter is appropriate.

While the embodiments for each variable have generally been listed above separately for each variable, the compounds are intended to include all combinations of embodiments described hereinabove including salts thereof.

Particular compounds of interest, and salts or solvates or stereoisomers thereof, include:
1-methylethyl((2S,4R)-1-acetyl-2-methyl-6-{1-[2-(methyloxy)ethyl]-1H-pyrazol-4-yl}-1,2,3,4-tetrahydro-4-quinolinyl)carbamate;
6-(((2S,4R)-1-acetyl-6-(1-(2-methoxyethyl)-1H-pyrazol-4-yl)-2-methyl-1,2,3,4-tetrahydroquinolin-4-yl)amino)nicotinonitrile;
6-(((2S,4R)-1-acetyl-6-(1-(2-methoxyethyl)-1H-1,2,3-triazol-4-yl)-2-methyl-1,2,3,4-tetrahydroquinolin-4-yl)amino)nicotinonitrile;
6-(((2S,4R)-1-acetyl-6-(2-(2-methoxyethyl)-2H-1,2,3-triazol-4-yl)-2-methyl-1,2,3,4-tetrahydroquinolin-4-yl)amino)nicotinonitrile;
1-((2S,4R)-4-((5-chloropyridin-2-yl)amino)-6-(1-(2-methoxyethyl)-1H-pyrazol-4-yl)-2-methyl-3,4-dihydroquinolin-1(2H)-yl);
1-((2S,4R)-6-(1-(2-methoxyethyl)-1H-pyrazol-4-yl)-2-methyl-4-((5-methylpyridin-2-yl)amino)-3,4-dihydroquinolin-1(2H)-yl)ethanone;
1-((2S,4R)-4-((3-chlorophenyl)amino)-6-(1-(2-methoxyethyl)-1H-pyrazol-4-yl)-2-methyl-3,4-dihydroquinolin-1(2H)-yl); and
1-((2S,4R)-6-(1-(2-methoxyethyl)-1H-pyrazol-4-yl)-2-methyl-4-((5-methylpyrazin-2-yl)amino)-3,4-dihydroquinolin-1(2H)-yl)ethanone.

In one of its composition aspects, the present embodiments provide a compound of formula IX:

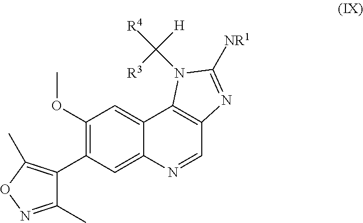

wherein

R¹ is hydrogen or C₁₋₃ alkyl and R² is selected from:
hydrogen;
C₁₋₆ alkyl; and
C₂₋₆ alkyl substituted by hydroxy, C₁₋₄ alkoxy or a group NR^{a}R^{b} in which R^{a} and R^{b} are independently hydrogen or C₁₋₄ alkyl, or R^{a} and R^{b} combined together with the N to which they are attached form a heterocyclyl ring; or

R¹ and R² combined together with the N to which they are attached form a heterocyclyl ring;

R³ is hydrogen, C₁₋₃ alkyl or —CH₂OH;

R⁴ is selected from:
a phenyl group optionally substituted by C₁₋₄ alkyl, CF₃, halogen, hydroxy or C₁₋₄ alkoxy;
a heteroaromatic group optionally substituted by C₁₋₄ alkyl, CF₃, halogen, hydroxy or C₁₋₄ alkoxy;
a tetrahydropyranyl group;
a tetrahydrofuranyl group;
a C₃₋₇ cycloalkyl group; and
a CH₂OMe group;

and salts or solvates or stereoisomers thereof.

In certain embodiments, R¹ and R² are combined together with the N to which they are attached to form a piperidinyl or morpholinyl ring.

In certain embodiments, R¹ is hydrogen and R² is hydrogen or C₁₋₄ alkyl (such as methyl, ethyl, isopropyl or isobutyl).

In certain embodiments, R¹ is hydrogen and R² is C₂₋₄ alkyl (such as methyl or ethyl) substituted by hydroxy or methoxy.

In certain embodiments, R¹ is hydrogen and R² is C₂₋₄ alkyl (such as ethyl) substituted by NR^{a}R^{b}, where R^{a} and R^{b} are both hydrogen or R^{a} and R^{b} are combined together with the N to which they are attached to form a morpholinyl ring.

In certain embodiments, R³ is hydrogen and R⁴ is tetrahydropyranyl.

In certain embodiments, R³ is hydrogen or methyl and R⁴ is pyridyl (such as 2-pyridyl).

In certain embodiments, R³ is hydrogen or methyl and R⁴ is pyrazolyl optionally substituted by C₁₋₄ alkyl.

In certain embodiments, R³ is hydrogen or methyl and R⁴ is phenyl.

In certain embodiments, R³ is methyl and R⁴ is —CH₂OMe.

Particular compounds of interest, and salts or solvates or stereoisomers thereof, include:
2-({7-(3,5-dimethyl-4-isoxazolyl)-8-(methyloxy)-1-[(1R)-1-phenylethyl]-1H-imidazo[4,5-c]quinolin-2-yl}amino)ethanol;
2-{[7-(3,5-dimethyl-4-isoxazolyl)-8-(methyloxy)-1-(tetrahydro-2H-pyran-4-ylmethyl)-1H-imidazo[4,5-c]quinolin-2-yl]amino}ethanol;
7-(3,5-dimethyl-4-isoxazolyl)-8-(methyloxy)-1-(tetrahydro-2H-pyran-4-ylmethyl)-1H-imidazo[4,5-c]quinolin-2-amine;
7-(3,5-dimethyl-4-isoxazolyl)-8-(methyloxy)-1-(2-pyridinylmethyl)-1H-imidazo[4,5-c]quinolin-2-amine;
7-(3,5-dimethyl-4-isoxazolyl)-1-[1-methyl-2-(methyloxy)ethyl]-8-(methyloxy)-1H-imidazo[4,5-c]quinolin-2-amine;
7-(3,5-dimethyl-4-isoxazolyl)-N-ethyl-8-(methyloxy)-1-(tetrahydro-2H-pyran-4-ylmethyl)-1H-imidazo[4,5-c]quinolin-2-amine;
7-(3,5-dimethylisoxazol-4-yl)-N-ethyl-8-methoxy-1-(1-methoxypropan-2-yl)-1H-imidazo[4,5-c]quinolin-2-amine;
N1-(7-(3,5-dimethylisoxazol-4-yl)-8-methoxy-1-((tetrahydro-2H-pyran-4-yl)methyl)-1H-imidazo[4,5-c]quinolin-2-yl)ethane-1,2-diamine;
7-(3,5-dimethyl-4-isoxazolyl)-8-(methyloxy)-N-[2-(4-morpholinyl)ethyl]-1-(tetrahydro-2H-pyran-4-ylmethyl)-1H-imidazo[4,5-c]quinolin-2-amine;
7-(3,5-dimethyl-4-isoxazolyl)-8-(methyloxy)-N-[2-(methyloxy)ethyl]-1-(tetrahydro-2H-pyran-4-ylmethyl)-1H-imidazo[4,5-c]quinolin-2-amine;
7-(3,5-dimethyl-4-isoxazolyl)-N-ethyl-1-[1-methyl-2-(methyloxy)ethyl]-8-(methyloxy)-1H-imidazo[4,5-c]quinolin-2-amine;
N-[7-(3,5-dimethyl-4-isoxazolyl)-1-[1-methyl-2-(methyloxy)ethyl]-8-(methyloxy)-1H-imidazo[4,5-c]quinolin-2-yl]-1,2-ethanediamine;
7-(3,5-dimethyl-4-isoxazolyl)-1-[1-methyl-2-(methyloxy)ethyl]-8-(methyloxy)-N-[2-(4-morpholinyl)ethyl]-1H-imidazo[4,5-c]quinolin-2-amine;
7-(3,5-dimethyl-4-isoxazolyl)-1-[1-methyl-2-(methyloxy)ethyl]-8-(methyloxy)-N-[2-(methyloxy)ethyl]-1H-imidazo[4,5-c]quinolin-2-amine;
7-(3,5-dimethyl-4-isoxazolyl)-8-(methyloxy)-N-[2-(methyloxy)ethyl]-1-(2-pyridinylmethyl)-1H-imidazo[4,5-c]quinolin-2-amine;
7-(3,5-dimethyl-4-isoxazolyl)-8-(methyloxy)-2-(4-morpholinyl)-1-[(1R)-1-phenylethyl]-1H-imidazo[4,5-c]quinoline;
7-(3,5-dimethyl-4-isoxazolyl)-8-(methyloxy)-1-[(1R)-1-phenylethyl]-2-(1-piperidinyl)-1H-imidazo[4,5-c]quinoline;
7-(3,5-dimethyl-4-isoxazolyl)-8-(methyloxy)-2-(4-morpholinyl)-1-(tetrahydro-2H-pyran-4-ylmethyl)-1H-imidazo[4,5-c]quinoline;
7-(3,5-dimethylisoxazol-4-yl)-N-ethyl-8-methoxy-1-(1-(1-methyl-1H-pyrazol-4-yl)ethyl)-1H-imidazo[4,5-c]quinolin-2-amine;
7-(3,5-dimethylisoxazol-4-yl)-8-methoxy-N,N-dimethyl-1-((tetrahydro-2H-pyran-4-yl)methyl)-1H-imidazo[4,5-c]quinolin-2-amine; and
7-(3,5-dimethylisoxazol-4-yl)-8-methoxy-N-(2-methoxyethyl)-1-((R)-1-(pyridin-2-yl)ethyl)-1H-imidazo[4,5-c]quinolin-2-amine.

In one of its composition aspects, the present embodiments provide a compound of formula X:

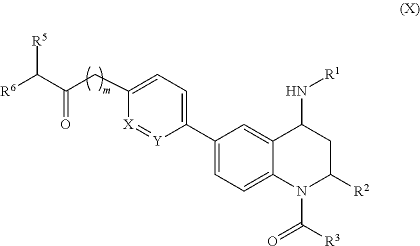

wherein

X and Y are independently CH or N provided that at least one of X and Y must be CH;

R¹ is a group —C(O)OR⁴ in which R⁴ is C₁₋₃ alkyl or C₃₋₇ cycloalkyl; or

R¹ is a group selected from phenyl, pyridyl, pyrazinyl and pyrimidinyl said groups being optionally substituted by one or two substituents selected from halogen, C₁₋₄ alkyl and CN;

R² is C₁₋₄ alkyl;

R³ is C₁₋₄alkyl;

R⁵ is hydrogen and R⁶ is C₁₋₄ alkyl substituted by one or more hydroxy or a —NR⁷R⁸ group in which R⁷ and R⁸ are independently hydrogen or a C₁₋₄ alkyl group; or

R⁵ and R⁶ together with the N to which they are attached form a 4, 5 or 6 membered heterocyclyl ring optionally containing a further heteroatom selected from N, O and S, said heterocyclyl ring being optionally substituted by one or more C₁₋₄ alkyl, hydroxyl or amino groups; and

m is 0, 1 or 2;

and salts or solvates or stereoisomers thereof.

In certain embodiments, compounds of formula (X) have cis relative stereochemistry across the tetrahydroquinoline ring in respect of the substituents in the 2 and 4 positions on the ring. In some embodiments, the compound of formula (X) or a salt thereof is the (2S, 4R) enantiomer.

In certain embodiments, X and Y are both CH. In some embodiments, X is CH and Y is N. In some embodiments, X is N and Y is CH.

In certain embodiments, R¹ is a group —C(O)OR⁴ in which R⁴ is isopropyl.

In certain embodiments, R¹ is selected from:

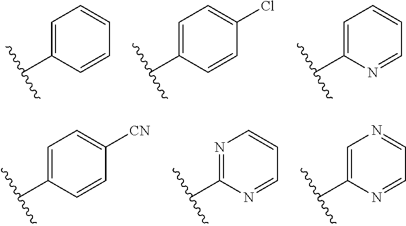

In certain embodiments, R is methyl.

In certain embodiments, R³ is methyl.

In certain embodiments, m is 0. In certain embodiments, m is 1. In certain embodiments, m is 2.

In certain embodiments, R⁵ is hydrogen and R⁶ is selected from:

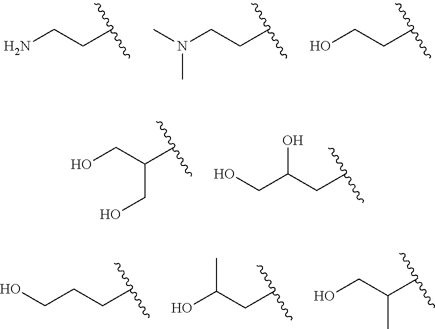

In certain embodiments, R⁵ and R⁶ together with the N to which they are attached form a 4, 5 or 6 membered heterocyclyl ring selected from azetidinyl, pyrrodinyl, piperazinyl, piperidinyl and morpholinyl, said heterocyclyl ring being optionally substituted by one or more (e.g., two or three) C₁₋₄ alkyl (such as methyl), hydroxyl, or amino groups.

In certain embodiments, R⁵ and R⁶ together with the N to which they are attached form group selected from:

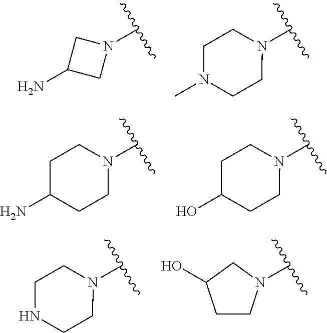

While the embodiments for each variable have generally been listed above separately for each variable, the present disclosure is intended to include all combinations of embodiments described hereinabove including salts thereof.

Particular compounds of interest, and salts or solvates or stereoisomers thereof, include:
2-(4-((2S,4R)-1-acetyl-4-((5-cyanopyridin-2-yl)amino)-2-methyl-1,2,3,4-tetrahydroquinolin-6-yl)phenyl)-N-(2-(dimethylamino)ethyl)acetamide;
6-((2S,4R)-1-acetyl-4((5-cyanopyridin-2-yl)amino)-2-methyl-1,2,3,4-tetrahydroquinolin-6-yl)-N-(2-(dimethylamino)ethyl)nicotinamide; and
6-((2S,4R)-1-acetyl-4-((5-cyanopyridin-2-yl)amino)-2-methyl-1,2,3,4-tetrahydroquinolin-6-yl)-N-(2-hydroxyethyl)nicotinamide;

or a salt thereof.

In one of its composition aspects, the present embodiments provide a compound of formula XI:

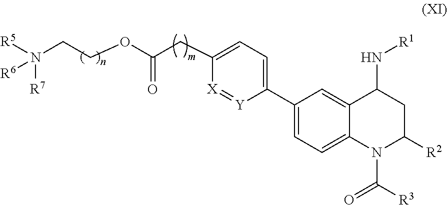

wherein

X and Y are independently CH or N provided that at least one of X and Y must be CH;

R¹ is a group —C(O)OR⁴ in which R⁴ is C₁₋₄ alkyl or C₃₋₇ cycloalkyl; or

R¹ is a group selected from phenyl, pyridyl, pyrazinyl and pyrimidinyl said groups being optionally substituted by one or two substituents selected from halogen, C₁₋₄ alkyl and CN;

R² is C₁₋₄ alkyl;

R³ is C₁₋₄ alkyl;

R⁵ and R⁶ are independently C₁₋₄ alkyl; or

R⁵ and R⁶ combined together with the N to which they are attached form a 5 or 6 membered heterocyclyl;

R⁷ is absent or is C₁₋₄ alkyl;

m is 0, 1 or 2; and

n is 1 or 2;

and salts or solvates or stereoisomers thereof.

In certain embodiments, compounds of formula (XI) have cis relative stereochemistry across the tetrahydroquinoline ring in respect of the substituents in the 2 and 4 positions on the ring. In some embodiments, the compound of formula (XI) or a salt thereof is the (2S, 4R) enantiomer.

In certain embodiments, X and Y are both CH. In certain embodiments, X is CH and Y is N.

In certain embodiments, R¹ is a group —C(O)OR⁴ in which R⁴ is isopropyl.

In certain embodiments, R¹ is phenyl or pyridyl optionally substituted by one or two substituents selected from halogen, C₁₋₄ alkyl and CN. In certain embodiments, R¹ is 4-chlorophenyl or R¹ is 5-cyanopyridin-2-yl.

In certain embodiments, R² is methyl.

In certain embodiments, R³ is methyl.

In certain embodiments, m is 0.

In certain embodiments, n is 0. In certain embodiments, n is 1.

In certain embodiments, R⁵ and R⁶ are both methyl.

It will be appreciated that when R⁷ is C₁₋₄ alkyl a quaternised ammonium moiety will be formed. In some embodiments, R⁷ is absent.

While the embodiments for each variable have generally been listed above separately for each variable, the present disclosure is intended to include all combinations of embodiments described hereinabove including salts thereof.

Particular compounds of interest, and salts or solvates or stereoisomers thereof, include:
2-(dimethylamino)ethyl-4-((2S,4R)-1-acetyl-4-((4-chlorophenyl)amino)-2-methyl-1,2,3,4-tetrahydroquinolin-6-yl)benzoate;
2-((4-((2S,4R)-1-acetyl-4-((4-chlorophenyl)amino)-2-methyl-1,2,3,4-tetrahydroquinolin-6-yl)benzoyl)oxy)-N,N,N-trimethylethanaminium;
3-((4-((2S,4R)-1-acetyl-4-((4-chlorophenyl)amino)-2-methyl-1,2,3,4-tetrahydroquinolin-6-yl)benzoyl)oxy)-N,N,N-trimethylpropan-1-aminium;
3-(dimethylamino)propyl-4-((2S,4R)-1-acetyl-4-((4-chlorophenyl)amino)-2-methyl-1,2,3,4-tetrahydroquinolin-6-yl)benzoate;
3-(dimethylamino)propyl-6-((2S,4R)-1-acetyl-4-((5-cyanopyridin-2-yl)amino)-2-methyl-1,2,3,4-tetrahydroquinolin-6-yl)nicotinate;
2-(dimethylamino)ethyl-6-((2R,4R)-1-acetyl-4-((5-cyanopyridin-2-yl)amino)-2-methyl-1,2,3,4-tetrahydroquinolin-6-yl)nicotinate;
3-(dimethylamino)propyl-4-((2S,4R)-1-acetyl-4-((isopropoxycarbonyl)amino)-2-methyl-1,2,3,4-tetrahydroquinolin-6-yl)benzoate; and
2-(dimethylamino)ethyl-4-((2S,4R)-1-acetyl-4-((isopropoxycarbonyl)amino)-2-methyl-1,2,3,4-tetrahydroquinolin-6-yl)benzoate;

or a salt thereof.

In one of its composition aspects, the present embodiments provide a compound of formula XII:

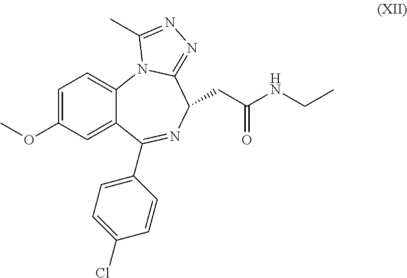 [SMILES: CCNC(=O)C[C@@H]1N=C(c2ccc(Cl)cc2)c2cc(OC)ccc2-n2c(C)nnc21]

((S)-2-(6-(4-chlorophenyl)-8-methoxy-1-methyl-4H-benzo[f][1,2,4]triazolo[4,3-a][1,4]diazepin-4-yl)-N-ethylacetamide),

and salts or solvates or stereoisomers thereof.

In one of its composition aspects, the present embodiments provide a compound of formula XIII:

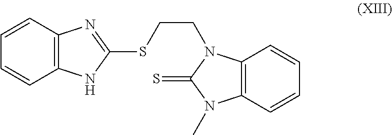 [SMILES: Cn1c(=S)n(CCSc2nc3ccccc3[nH]2)c2ccccc21]

(1-(2-(1H-benzo[d]imidazol-2-ylthio)ethyl)-3-methyl-1H-benzo[d]imidazole-2(3H)-thione),

and salts or solvates or stereoisomers thereof.

In one of its composition aspects, the present embodiments provide a compound of formula XIV:

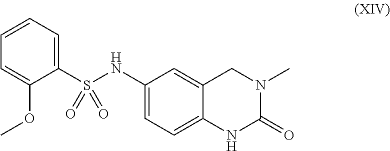 [SMILES: COc1ccccc1S(=O)(=O)Nc1ccc2c(c1)CN(C)C(=O)N2]

(2-methoxy-N-(3-methyl-2-oxo-1,2,3,4-tetrahydroquinazolin-6-yl)benzenesulfonamide),

and salts or solvates or stereoisomers thereof.

Additional compounds for reactivating latent immunodeficiency virus according to embodiments of the present disclosure are found in, e.g., WO2012/116170; WO2012/069525; WO2012/055880; WO2012/055879; WO2011/143651; WO2011/054848; WO2011/054846; WO2011/54845; WO2011/054843; and WO2011/054553, the disclosures of each of which are incorporated herein by reference.

### Formulations, Dosages, and Routes of Administration

In general, an active agent (e.g., a compound that binds a BRD and activates immunodeficiency virus transcription; also referred to herein as a “BRD inhibitor”) is prepared in a pharmaceutically acceptable composition(s) for delivery to a host. In the context of reducing immunodeficiency virus transcription, the terms “active agent,” “drug,” “agent,” “therapeutic agent,” and the like are used interchangeably herein to refer to a compound that binds a BRD and activates immunodeficiency virus transcription, or an active ester thereof.

Pharmaceutically acceptable carriers preferred for use with active agents (and optionally one or more additional therapeutic agent) may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, and microparticles, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition comprising an active agent (and optionally one or more additional therapeutic agent) may also be lyophilized using means well known in the art, for subsequent reconstitution and use according to the invention.

### Formulations

An active agent is administered to an individual in need thereof in a formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds. 3^{rd} ed. Amer. Pharmaceutical Assoc. For the purposes of the following description of formulations, “active agent” includes an active agent as described above, and optionally one or more additional therapeutic agent.

In a subject method, an active agent may be administered to the host using any convenient means capable of resulting in the desired degree of reduction of immunodeficiency virus transcription. Thus, an active agent can be incorporated into a variety of formulations for therapeutic administration. For example, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In an exemplary embodiment, an active agent is formulated as a gel, as a solution, or in some other form suitable for intravaginal administration. In a further exemplary embodiment, an active agent is formulated as a gel, as a solution, or in some other form suitable for rectal (e.g., intrarectal) administration.

In pharmaceutical dosage forms, an active agent may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, an active is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the active agent(s) in a composition as a solution in sterile water, normal saline or another pharmaceutically acceptable carrier.

Unit dosage forms for intravaginal or intrarectal administration such as syrups, elixirs, gels, and suspensions may be provided wherein each dosage unit, for example, teaspoonful, tablespoonful, tablet, unit gel volume, or suppository, contains a predetermined amount of the composition containing one or more active agents.

The term “unit dosage form,” as used herein, refers to physically discrete units suitable as unitary dosages for human and animal subjects, each unit containing a predetermined quantity of an active agent, calculated in an amount sufficient to produce the desired effect in association with a pharmaceutically acceptable diluent, carrier or vehicle. The specifications for a given active agent will depend in part on the particular compound employed and the effect to be achieved, and the pharmacodynamics associated with each compound in the host.

Other modes of administration will also find use with the subject invention. For instance, an active agent can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g. about 1% to about 2%.

An active agent can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

An active agent will in some embodiments be formulated for vaginal delivery. A subject formulation for intravaginal administration comprises an active agent formulated as an intravaginal bioadhesive tablet, intravaginal bioadhesive microparticle, intravaginal cream, intravaginal lotion, intravaginal foam, intravaginal ointment, intravaginal paste, intravaginal solution, or intravaginal gel.

An active agent will in some embodiments be formulated for rectal delivery. A subject formulation for intrarectal administration comprises an active agent formulated as an intrarectal bioadhesive tablet, intrarectal bioadhesive microparticle, intrarectal cream, intrarectal lotion, intrarectal foam, intrarectal ointment, intrarectal paste, intrarectal solution, or intrarectal gel.

A subject formulation comprising an active agent includes one or more of an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Tablets comprising an active agent may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and the like.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Dosages

Although the dosage used will vary depending on the clinical goals to be achieved, a suitable dosage range of an active agent is one which provides up to about 1 mg to about 1000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, or from about 500 mg to about 1000 mg of an active agent can be administered in a single dose.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, a single dose of an active agent is administered. In other embodiments, multiple doses of an active agent are administered. Where multiple doses are administered over a period of time, an active agent is administered twice daily (qid), daily (qd), every other day (qod), every third day, three times per week (tiw), or twice per week (biw) over a period of time. For example, an active agent is administered qid, qd, qod, tiw, or biw over a period of from one day to about 2 years or more. For example, an active agent is administered at any of the aforementioned frequencies for one week, two weeks, one month, two months, six months, one year, or two years, or more, depending on various factors.

Where two different active agents are administered, a first active agent and a second active agent can be administered in separate formulations. A first active agent and a second active agent can be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

### Routes of Administration

An active agent is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, transdermal, subcutaneous, intradermal, topical application, intravenous, vaginal, nasal, and other parenteral routes of administration. In some embodiments, an active agent is administered via an intravaginal route of administration. In other embodiments, an active agent is administered via an intrarectal route of administration. Routes of administration may be combined, if desired, or adjusted depending upon the agent and/or the desired effect. The composition can be administered in a single dose or in multiple doses.

An active agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, vaginal, transdermal, subcutaneous, intramuscular, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

An active agent can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as the number of viral particles per unit blood. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class mammalia, and primates (e.g., humans, chimpanzees, and monkeys), that are susceptible to immunodeficiency virus (e.g., HIV) infection. In many embodiments, the hosts will be humans.

### Kits, Containers, Devices, Delivery Systems

Kits with unit doses of the active agent, e.g. in oral, vaginal, rectal, transdermal, or injectable doses (e.g., for intramuscular, intravenous, or subcutaneous injection), are provided. In such kits, in addition to the containers containing the unit doses will be an informational package insert describing the use and attendant benefits of the drugs in treating an immunodeficiency virus (e.g., an HIV) infection. Suitable active agents and unit doses are those described herein above.

In many embodiments, a subject kit will further include instructions for practicing the subject methods or means for obtaining the same (e.g., a website URL directing the user to a webpage which provides the instructions), where these instructions are typically printed on a substrate, which substrate may be one or more of: a package insert, the packaging, formulation containers, and the like.

In some embodiments, a subject kit includes one or more components or features that increase patient compliance, e.g., a component or system to aid the patient in remembering to take the active agent at the appropriate time or interval. Such components include, but are not limited to, a calendaring system to aid the patient in remembering to take the active agent at the appropriate time or interval.

The present invention provides a delivery system comprising an active agent that inhibits LSD1 enzymatic activity. In some embodiments, the delivery system is a delivery system that provides for injection of a formulation comprising an active agent subcutaneously, intravenously, or intramuscularly. In other embodiments, the delivery system is a vaginal or rectal delivery system.

In some embodiments, an active agent is packaged for oral administration. The present invention provides a packaging unit comprising daily dosage units of an active agent. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

In some embodiments, a subject delivery system comprises an injection device. Exemplary, non-limiting drug delivery devices include injections devices, such as pen injectors, and needle/syringe devices. In some embodiments, the invention provides an injection delivery device that is pre-loaded with a formulation comprising an effective amount of a BRD inhibitor. For example, a subject delivery device comprises an injection device pre-loaded with a single dose of a BRD inhibitor. A subject injection device can be re-usable or disposable.

Pen injectors are well known in the art. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

The present invention provides a delivery system for vaginal or rectal delivery of an active agent to the vagina or rectum of an individual. The delivery system comprises a device for insertion into the vagina or rectum. In some embodiments, the delivery system comprises an applicator for delivery of a formulation into the vagina or rectum; and a container that contains a formulation comprising an active agent. In these embodiments, the container (e.g., a tube) is adapted for delivering a formulation into the applicator. In other embodiments, the delivery system comprises a device that is inserted into the vagina or rectum, which device includes an active agent. For example, the device is coated with, impregnated with, or otherwise contains a formulation comprising the active agent.

In some embodiments, the vaginal or rectal delivery system is a tampon or tampon-like device that comprises a subject formulation. Drug delivery tampons are known in the art, and any such tampon can be used in conjunction with a subject drug delivery system. Drug delivery tampons are described in, e.g., U.S. Pat. No. 6,086,909. If a tampon or tampon-like device is used, there are numerous methods by which an active agent can be incorporated into the device. For example, the drug can be incorporated into a gel-like bioadhesive reservoir in the tip of the device. Alternatively, the drug can be in the form of a powdered material positioned at the tip of the tampon. The drug can also be absorbed into fibers at the tip of the tampon, for example, by dissolving the drug in a pharmaceutically acceptable carrier and absorbing the drug solution into the tampon fibers. The drug can also be dissolved in a coating material which is applied to the tip of the tampon. Alternatively, the drug can be incorporated into an insertable suppository which is placed in association with the tip of the tampon.

In other embodiments, the drug delivery device is a vaginal or rectal ring. Vaginal or rectal rings usually consist of an inert elastomer ring coated by another layer of elastomer containing an active agent to be delivered. The rings can be easily inserted, left in place for the desired period of time (e.g., up to 7 days), then removed by the user. The ring can optionally include a third, outer, rate-controlling elastomer layer which contains no drug. Optionally, the third ring can contain a second drug for a dual release ring. The drug can be incorporated into polyethylene glycol throughout the silicone elastomer ring to act as a reservoir for drug to be delivered.

In other embodiments, a subject vaginal or rectal delivery system is a vaginal or rectal sponge. The active agent is incorporated into a silicone matrix which is coated onto a cylindrical drug-free polyurethane sponge, as described in the literature.

Pessaries, tablets, and suppositories are other examples of drug delivery systems which can be used, e.g., in carrying out a method of the present disclosure. These systems have been described extensively in the literature.

Bioadhesive microparticles constitute still another drug delivery system suitable for use in the present invention. This system is a multi-phase liquid or semi-solid preparation which does not seep from the vagina or rectum as do many suppository formulations. The substances cling to the wall of the vagina or rectum and release the drug over a period of time. Many of these systems were designed for nasal use but can be used in the vagina or rectum as well (e.g. U.S. Pat. No. 4,756,907). The system may comprise microspheres with an active agent; and a surfactant for enhancing uptake of the drug. The microparticles have a diameter of 10-100 μm and can be prepared from starch, gelatin, albumin, collagen, or dextran.

Another system is a container comprising a subject formulation (e.g., a tube) that is adapted for use with an applicator. The active agent is incorporated into creams, lotions, foams, paste, ointments, and gels which can be applied to the vagina or rectum using an applicator. Processes for preparing pharmaceuticals in cream, lotion, foam, paste, ointment and gel formats can be found throughout the literature. An example of a suitable system is a standard fragrance free lotion formulation containing glycerol, ceramides, mineral oil, petrolatum, parabens, fragrance and water such as the product sold under the trademark JERGENS™ (Andrew Jergens Co., Cincinnati, Ohio). Suitable nontoxic pharmaceutically acceptable systems for use in the compositions of the present invention will be apparent to those skilled in the art of pharmaceutical formulations and examples are described in Remington's Pharmaceutical Sciences, 19th Edition, A. R. Gennaro, ed., 1995. The choice of suitable carriers will depend on the exact nature of the particular vaginal or rectal dosage form desired, e.g., whether the active ingredient(s) is/are to be formulated into a cream, lotion, foam, ointment, paste, solution, or gel, as well as on the identity of the active ingredient(s). Other suitable delivery devices are those described in U.S. Pat. No. 6,476,079.

### Combination Therapy

In some embodiments, a BRD inhibitor is administered in combination therapy with one or more additional therapeutic agents. Suitable additional therapeutic agents include agents that inhibit one or more functions of an immunodeficiency virus; agents that treat or ameliorate a symptom of an immunodeficiency virus infection; agents that treat an infection that occurs secondary to an immunodeficiency virus infection; and the like.

Therapeutic agents include, e.g., beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, interferon, zidovudine (AZT, Retrovir™), didanosine (dideoxyinosine, ddI, Videx™), stavudine (d4T, Zerit™), zalcitabine (dideoxycytosine, ddC, Hivid™), nevirapine (Viramune™), lamivudine (Epivir™, 3TC), protease inhibitors, saquinavir (Invirase™, Fortovase™), ritonavir (Norvir™), nelfinavir (Viracept™), efavirenz (Sustiva™) abacavir (Ziagen™), amprenavir (Agenerase™) indinavir (Crixivan™), ganciclovir, AzDU, delavirdine (Rescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof. Anti-HIV agents are those in the preceding list that specifically target a function of one or more HIV proteins.

In some embodiments, a BRD inhibitor is administered in combination therapy with two or more anti-HIV agents. For example, a BRD inhibitor can be administered in combination therapy with one, two, or three nucleoside reverse transcriptase inhibitors (e.g., Combivir, Epivir, Hivid, Retrovir, Videx, Zerit, Ziagen, etc.). A BRD inhibitor can be administered in combination therapy with one or two non-nucleoside reverse transcriptase inhibitors (e.g., Rescriptor, Sustiva, Viramune, etc.). A BRD inhibitor can be administered in combination therapy with one or two protease inhibitors (e.g., Agenerase, Crixivan, Fortovase, Invirase, Kaletra, Norvir, Viracept, etc.). A BRD inhibitor can be administered in combination therapy with a protease inhibitor and a nucleoside reverse transcriptase inhibitor. A BRD inhibitor can be administered in combination therapy with a protease inhibitor, a nucleoside reverse transcriptase inhibitor, and a non-nucleoside reverse transcriptase inhibitor. A BRD inhibitor can be administered in combination therapy with a protease inhibitor and a non-nucleoside reverse transcriptase inhibitor. Other combinations of a BRD inhibitor with one or more of a protease inhibitor, a nucleoside reverse transcriptase inhibitor, and a non-nucleoside reverse transcriptase inhibitor are contemplated.

In some embodiments, a subject treatment method involves administering: a) a BRD inhibitor; and b) an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

In some embodiments, a subject treatment method involves administering: a) a BRD inhibitor; and b) an HIV inhibitor, where suitable HIV inhibitors include, but are not limited to, one or more nucleoside/nucleotide reverse transcriptase inhibitors (NRTIs), non-nucleoside reverse transcriptase inhibitors (NNRTIs), protease inhibitors (PIs), fusion inhibitors, integrase inhibitors, chemokine receptor (e.g., CXCR4, CCR5) inhibitors, and hydroxyurea.

Nucleoside reverse transcriptase inhibitors include, but are not limited to, abacavir (ABC; ZIAGEN™), didanosine (dideoxyinosine (ddI); VIDEX™), lamivudine (3TC; EPIVIR™), stavudine (d4T; ZERIT™, ZERIT XR™), zalcitabine (dideoxycytidine (ddC); HIVID™), zidovudine (ZDV, formerly known as azidothymidine (AZT); RETROVIR™), abacavir, zidovudine, and lamivudine (TRIZIVIR™), zidovudine and lamivudine (COMBIVIR™), and emtricitabine (EMTRIVA™). Nucleotide reverse transcriptase inhibitors include tenofovir disoproxil fumarate (VIREAD™). Non-nucleoside reverse transcriptase inhibitors for HIV include, but are not limited to, nevirapine (VIRAMUNE™), delavirdine mesylate (RESCRIPTOR™), and efavirenz (SUSTIVA™).

Protease inhibitors (PIs) for treating HIV infection include amprenavir (AGENERASE™), saquinavir mesylate (FORTOVASE™, INVIRASE™.), ritonavir (NORVIR™), indinavir sulfate (CRIXIVAN™), nelfmavir mesylate (VIRACEPT™), lopinavir and ritonavir (KALETRA™), atazanavir (REYATAZ™), and fosamprenavir (LEXIVA™)

Fusion inhibitors prevent fusion between the virus and the cell from occurring, and therefore, prevent HIV infection and multiplication. Fusion inhibitors include, but are not limited to, enfuvirtide (FUZEON™), Lalezari et al., New England J. Med., 348:2175-2185 (2003); and maraviroc (SELZENTRY™, Pfizer).

An integrase inhibitor blocks the action of integrase, preventing HIV-1 genetic material from integrating into the host DNA, and thereby stopping viral replication. Integrase inhibitors include, but are not limited to, raltegravir (ISENTRESS™, Merck); and elvitegravir (GS 9137, Gilead Sciences).

Maturation inhibitors include, e.g., bevirimat (3β-(3-carboxy-3-methyl-butanoyloxy) lup-20(29)-en-28-oic acid); and Vivecon (MPC9055).

In some embodiments, a subject treatment method involves administering: a) a BRD inhibitor; and b) one or more of: (1) an HIV protease inhibitor selected from amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, GW640385X, DG17, PPL-100, DG35, and AG 1859; (2) an HIV non-nucleoside inhibitor of reverse transcriptase selected from capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirene), efavirenz, BILR 355 BS, VRX 840773, UK-453061, and RDEA806; (3) an HIV nucleoside inhibitor of reverse transcriptase selected from zidovudine, emtricitabine, didanosine, stavudine, zalcitabine, lamivudine, abacavir, amdoxovir, elvucitabine, alovudine, MIV-210, racivir, D-d4FC, emtricitabine, phosphazide, fozivudine tidoxil, apricitibine (AVX754), amdoxovir, KP-1461, and fosalvudine tidoxil (formerly HDP 99.0003); (4) an HIV nucleotide inhibitor of reverse transcriptase selected from tenofovir and adefovir; (5) an HIV integrase inhibitor selected from curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), BMS-538158, GSK364735C, BMS-707035, MK-2048, and BA 011; (6) a gp41 inhibitor selected from enfuvirtide, sifuvirtide, FB006M, and TRI-1144; (7) a CXCR4 inhibitor, such as AMD-070; (8) an entry inhibitor, such as SP01A; (9) a gp120 inhibitor, such as BMS-488043 and/or BlockAide/CR; (10) a G6PD and NADH-oxidase inhibitor, such as immunitin; (11) a CCR5 inhibitors selected from the group consisting of aplaviroc, vicriviroc, maraviroc, PRO-140, INCB15050, PF-232798 (Pfizer), and CCR5 mAb004; (12) another drug for treating HIV selected from BAS-100, SPI-452, REP 9, SP-01A, TNX-355, DES6, ODN-93, ODN-112, VGV-1, PA-457 (bevirimat), Ampligen, HRG214, Cytolin, VGX-410, KD-247, AMZ 0026, CYT 99007A-221 HIV, DEBIO-025, BAY 50-4798, MDXO10 (ipilimumab), PBS119, ALG 889, and PA-1050040 (PA-040); (13) any combinations or mixtures of the above.

As further examples, in some embodiments, a subject treatment method involves administering: a) a BRD inhibitor; and b) one or more of: i) amprenavir (Agenerase; (3S)-oxolan-3-yl N-[(2S,3R)-3-hydroxy-4-[N-(2-methylpropyl)(4-aminobenzene)sulfonamido]-1-phenylbutan-2-yl]carbamate) in an amount of 600 mg or 1200 mg twice daily; ii) tipranavir (Aptivus; N-{3-[(1R)-1-[(2R)-6-hydroxy-4-oxo-2-(2-phenylethyl)-2-propyl-3,4-dihydro-2H-pyran-5-yl]propyl]phenyl}-5-(trifluoromethyl)pyridine-2-sulfonamide) in an amount of 500 mg twice daily; iii) idinavir (Crixivan; (2S)-1-[(2S,4R)-4-benzyl-2-hydroxy-4-{[(1S,2R)-2-hydroxy-2,3-dihydro-1H-inden-1-yl]carbamoyl}butyl]-N-tert-butyl-4-(pyridin-3-ylmethyl)piperazine-2-carboxamide) in an amount of 800 mg three times daily; iv) saquinavir (Invirase; 2S)—N-[(2S,3R)-4-[(3S)-3-(tert-butylcarbamoyl)-decahydroisoquinolin-2-yl]-3-hydroxy-1-phenylbutan-2-yl]-2-(quinolin-2-ylformamido)butanediamide) in an amount of 1,000 mg twice daily; v) lopinavir and ritonavir (Kaleta; where lopinavir is 2S)—N-[(2S,4S,5S)-5-[2-(2,6-dimethylphenoxyl)acetamido]-4-hydroxy-1,6-diphenylhexan-2-yl]-3-methyl-2-(2-oxo-1,3-diazinan-1-yl)butanamide; and ritonavir is 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate) in an amount of 133 mg twice daily; vi) fosamprenavir (Lexiva; {[(2R,3S)-1-[N-(2-methylpropyl)(4-aminobenzene)sulfonamido]-3-({[(3S)-oxolan-3-yloxy]carbonyl}amino)-4-phenylbutan-2-yl]oxy}phosphonic acid) in an amount of 700 mg or 1400 mg twice daily); vii) ritonavir (Norvir) in an amount of 600 mg twice daily; viii) nelfinavir (Viracept; (3S,4aS,8aS)—N-tert-butyl-2-[(2R,3R)-2-hydroxy-3-[(3-hydroxy-2-methylphenyl)formamido]-4-(phenylsulfanyl)butyl]-decahydroisoquinoline-3-carboxamide) in an amount of 750 mg three times daily or in an amount of 1250 mg twice daily; ix) Fuzeon (Acetyl-YTSLIHSLIEESQNQ QEKNEQELLELDKWASLWNWF-amide; SEQ ID NO:8) in an amount of 90 mg twice daily; x) Combivir in an amount of 150 mg lamivudine (3TC; 2′,3′-dideoxy-3′-thiacytidine) and 300 mg zidovudine (AZT; azidothymidine) twice daily; xi) emtricitabine (Emtriva; 4-amino-5-fluoro-1-[(2R,5S)-2-(hydroxymethyl)-1,3-oxathiolan-5-yl]-1,2-dihydropyrimidin-2-one) in an amount of 200 mg once daily; xii) Epzicom in an amount of 600 mg abacavir (ABV; {(1S,4R)-4-[2-amino-6-(cyclopropylamino)-9H-purin-9-yl]cyclopent-2-en-1-yl}methanol) and 300 mg 3TC once daily; xiii) zidovudine (Retrovir; AZT or azidothymidine) in an amount of 200 mg three times daily; xiv) Trizivir in an amount of 150 mg 3TC and 300 mg ABV and 300 mg AZT twice daily; xv) Truvada in an amount of 200 mg emtricitabine and 300 mg tenofovir (({[(2R)-1-(6-amino-9H-purin-9-yl)propan-2-yl]oxy}methyl)phosphonic acid) once daily; xvi) didanosine (Videx; 2′,3′-dideoxyinosine) in an amount of 400 mg once daily; xvii) tenofovir (Viread) in an amount of 300 mg once daily; xviii) abacavir (Ziagen) in an amount of 300 mg twice daily; xix) atazanavir (Reyataz; methyl N-[(1S)-1-{[(2S,3S)-3-hydroxy-4-[(2S)-2-[(methoxycarbonyl)amino]-3,3-dimethyl-N′-{[4-(pyridin-2-yl)phenyl]methyl}butanehydrazido]-1-phenylbutan-2-yl]carbamoyl}-2,2-dimethylpropyl]carbamate) in an amount of 300 mg once daily or 400 mg once daily; xx) lamivudine (Epivir) in an amount of 150 mg twice daily; xxi) stavudine (Zerit; 2′-3′-didehydro-2′-3′-dideoxythymidine) in an amount of 40 mg twice daily; xxii) delavirdine (Rescriptor; N-[2-({4-[3-(propan-2-ylamino)pyridin-2-yl]piperazin-1-yl}carbonyl)-1H-indol-5-yl]methanesulfonamide) in an amount of 400 mg three times daily; xxiii) efavirenz (Sustiva; (4S)-6-chloro-4-(2-cyclopropylethynyl)-4-(trifluoromethyl)-2,4-dihydro-1H-3,1-benzoxazin-2-one) in an amount of 600 mg once daily); xxiv) nevirapine (Viramune; 11-cyclopropyl-4-methyl-5,11-dihydro-6H-dipyrido[3,2-b:2′,3′-e][1,4]diazepin-6-one) in an amount of 200 mg twice daily); xxv) bevirimat; and xxvi) Vivecon.

In some embodiments, a subject treatment method involves administering: a) a BRD inhibitor; and b) a PKC activator. An example of a suitable PKC activator is prostratin ((1aR,1bS,4aR,7aS,7bR,8R,9aS)-4a,7b-dihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl). The PKC activator can be administered in a separate formulation from a BRD inhibitor. A PKC activator can be co-formulated with a BRD inhibitor, and the co-formulation administered to an individual. The present disclosure provides a kit comprising a PKC activator in a first container; and a BRD inhibitor in a second container.

### Subjects Suitable for Treatment

The methods of the present disclosure are suitable for treating individuals who have an immunodeficiency virus infection, e.g., who have been diagnosed as having an immunodeficiency virus infection.

The methods of the present disclosure are suitable for treating individuals who have an HIV infection (e.g., who have been diagnosed as having an HIV infection), and individuals who are at risk of contracting an HIV infection. Such individuals include, but are not limited to, individuals with healthy, intact immune systems, but who are at risk for becoming HIV infected (“at-risk” individuals). At-risk individuals include, but are not limited to, individuals who have a greater likelihood than the general population of becoming HIV infected. Individuals at risk for becoming HIV infected include, but are not limited to, individuals at risk for HIV infection due to sexual activity with HIV-infected individuals. Individuals suitable for treatment include individuals infected with, or at risk of becoming infected with, HIV-1 and/or HIV-2 and/or HIV-3, or any variant thereof.

### Detection Methods

The present disclosure provides detection methods for identifying a cell that has latent HIV. The methods generally involve contacting a cell obtained from an individual with a bromodomain (BRD) inhibitor; and detecting expression of an HIV-encoded gene product. If the cell expresses an HIV-encoded gene product when contacted with the BRD inhibitor, but does not express detectable levels of the HIV-encoded gene product in the absence of the BRD inhibitor, the cell is considered to harbor latent HIV (i.e., to have latent HIV present in the cell genome). Thus, a subject detection method can comprise contacting a cell obtained from an individual with a BRD inhibitor; detecting expression of an HIV-encoded gene product; and comparing the expression, if any, of the HIV-encoded gene product in the cell contacted with the BRD inhibitor with expression of the HIV-encoded gene product in a control cell not contacted with the BRD inhibitor.

Cells obtained from an individual include cells in a liquid cell suspension sample, and cells in a solid tissue sample. A cell sample obtained from an individual can be from any of a variety of tissues, e.g., brain, blood, saliva, muscle, liver, bronchoalveolar lavage, sputum, etc. The cells can be obtained in any of a variety of forms, e.g., in a buccal swab, in a blood sample, or in any type of tissue biopsy. The cell sample can be obtained from a living individual. The cell sample can be a post-mortem sample. Cells present in the cell sample can be living cells.

A cell in a cell sample obtained from an individual is contacted with a BRD inhibitor; and expression of an HIV-encoded gene product is detected. Gene products include nucleic acids (e.g., mRNA) and protein.

Methods of detecting nucleic acid gene products are well known in the art; any such method can be used in a subject detection method. For example, a hybridization method can be used, using a suitably labeled nucleic acid probe. Detection can be accomplished by any known method, including, but not limited to, in situ hybridization, PCR, RT-PCR, and “Northern” or RNA blotting, or combinations of such techniques, using a suitably labeled nucleic acid probe.

In some cases, a polymerase chain reaction (PCR) method (e.g., a reverse transcription-PCR method; a quantitative PCR method; etc.) is used, employing primers (e.g., pairs of primer oligonucleotides) that amplify an HIV gene. The primer nucleic acids are prepared using any known method, e.g., automated synthesis, and the like. The primer pairs are chosen such that they specifically amplify a cDNA copy of an mRNA encoding an HIV polypeptide.

Methods using PCR amplification can be performed on the DNA from a single cell, although it is convenient to use at least about 10⁵ cells. A detectable label may be included in the amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high-affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

A number of methods are available for determining the expression level of a gene or protein in a particular sample. For example, detection may utilize staining of cells or histological sections with labeled antibodies, performed in accordance with conventional methods. Cells are permeabilized to stain cytoplasmic molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g. fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc.

Methods of detecting polypeptide gene products are known in the art, and include, e.g., immunological assays such as an enzyme-linked immunosorbent assay (ELISA), a protein blot assay, a radioimmunoassay, and the like, where such assays employ an antibody specific for an HIV-encoded polypeptide.

A subject detection method can be used to detect the presence, in a cell sample obtained from an individual, of a cell harboring latent HIV. In some cases, detection in a cell sample obtained from a living individual of a cell harboring latent HIV may indicate that the individual should be treated with an agent that reactivates latent HIV. For example, the individual may be undergoing treatment for an HIV infection at the time the individual is subjected to a subject detection method; in such cases, the individual may be treated with both a treatment regimen for treating the HIV infection, and with an agent that reactivates latent HIV.

A subject detection method can be used to isolate primary cells harboring latent HIV. Such cells can be used in a subject screening method, as described below.

In some cases, a subject detection method further comprises isolating a cell that has been identified as harboring latent HIV in its genome.

### Screening Methods

The present disclosure provides a method of identifying a candidate agent for treating an HIV infection in an individual. The method generally involves contacting a primary cell identified using a subject detection method with a bromodomain (BRD) inhibitor and a test agent; and determining the effect of the test agent on the level of HIV produced in the cell and/or the level of an HIV-encoded gene product in the cell. A test agent that reduces the level of HIV produced in the cell and/or the level of production of an HIV-encoded gene product, compared to the level of HIV produced and/or the level of an HIV-encoded gene product in a control cell contacted with the BRD inhibitor but not with the test agent, is considered a candidate agent for inhibiting HIV and treating an HIV infection.

A test agent that reduces level of HIV produced in the cell and/or the level of production of an HIV-encoded gene product by at least about 10%, at least about 20%, at least about 25%, at least about 30%, at least about 40%, at least about 50%, at least about 60%, at least about 70%, at least about 80%, or more than 80%, is considered a candidate agent for treating an HIV infection.

As used herein, the term “determining” refers to both quantitative and qualitative determinations and as such, the term “determining” is used interchangeably herein with “assaying,” “measuring,” and the like.

The terms “candidate agent,” “test agent,” “agent”, “substance” and “compound” are used interchangeably herein. Candidate agents encompass numerous chemical classes, typically synthetic, semi-synthetic, or naturally occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.) and can also be used. Alternatively, libraries of natural compounds in the form of bacterial, fungal, plant and animal extracts are available from Pan Labs (Bothell, Wash.) or are readily producible.

Candidate agents can be small organic or inorganic compounds having a molecular weight of more than 50 and less than about 2,500 daltons. Candidate agents can comprise functional groups necessary for structural interaction with proteins, e.g., hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, and derivatives, structural analogs or combinations thereof.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Activation of Latent HIV with BRD Inhibitors

Compounds that specifically bind to the bromodomain of PCAF in vitro were tested in an in vivo assay that measures the transactivation of the HIV promoter by Tat. The Jurkat 1G5 cells contain a stably integrated and silent HIV LTR-luciferase reporter construct. These cells were infected with an HIV-based retroviral vector expressing a 101-amino acids Tat (Tat2ex), generating the 1G5-Tat cells. Introduction of Tat protein activates HIV transcription and produces a stable increase in luciferase activity. Clonal cell lines were derived from the original population and have characterized them in terms of luciferase expression and response to Tat inhibitors. Luciferase expression is stable over time in each clone and is inhibited by more than 80% in response to DRB, a known inhibitor of CDK9 kinase activity and Tat transactivation.

Compounds were tested by incubating them at various concentrations ranging from 1.25 to 40 μM on either 1G5 cells, to test for effects on the HIV LTR, or on 1G5-Tat cells to test for effects on Tat-induced HIV transcription. After 24 hr, cells were harvested and processed for luciferase activity measurement.

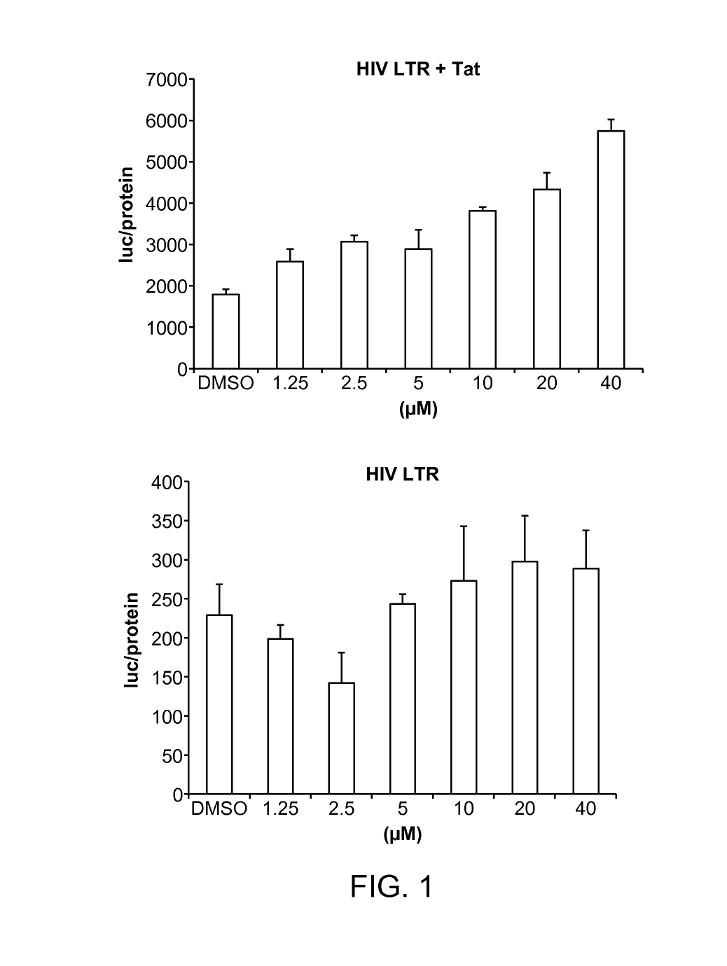

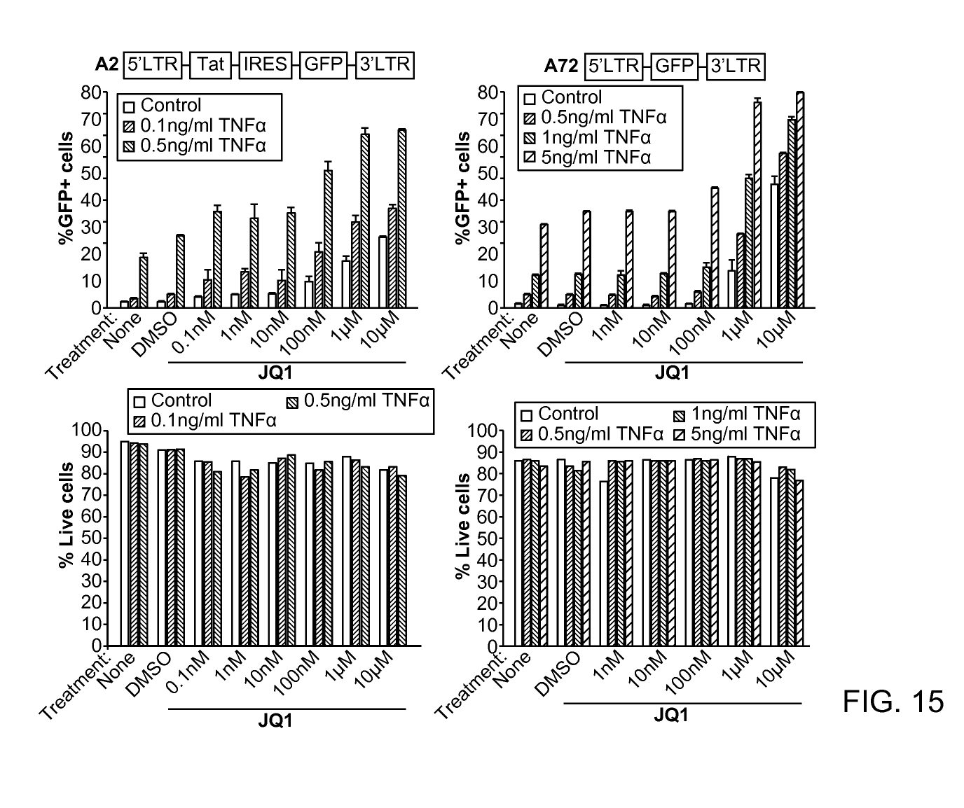

Additional cell lines, comprising latent HIV constructs, were used to test compounds. The results are shown in FIGS. 1-15. Examples of cell lines and constructs are depicted schematically in FIG. 15.

FIG. 1 depicts the effect of Compound 5110065 on the activity of the HIV promoter in the presence (HIV LTR+Tat) and absence (HIV LTR) of Tat. The experiments used Jurkat 1G5 cells, which contain a stably integrated HIV LTR luciferase vector; and Jurkat 1G5-Tat cells, which are further infected with a lentivirus expressing Tat. The concentration of compound was varied from 1.25 μM to 40 μM and luciferase activity was used as a measurement of HIV transcription.

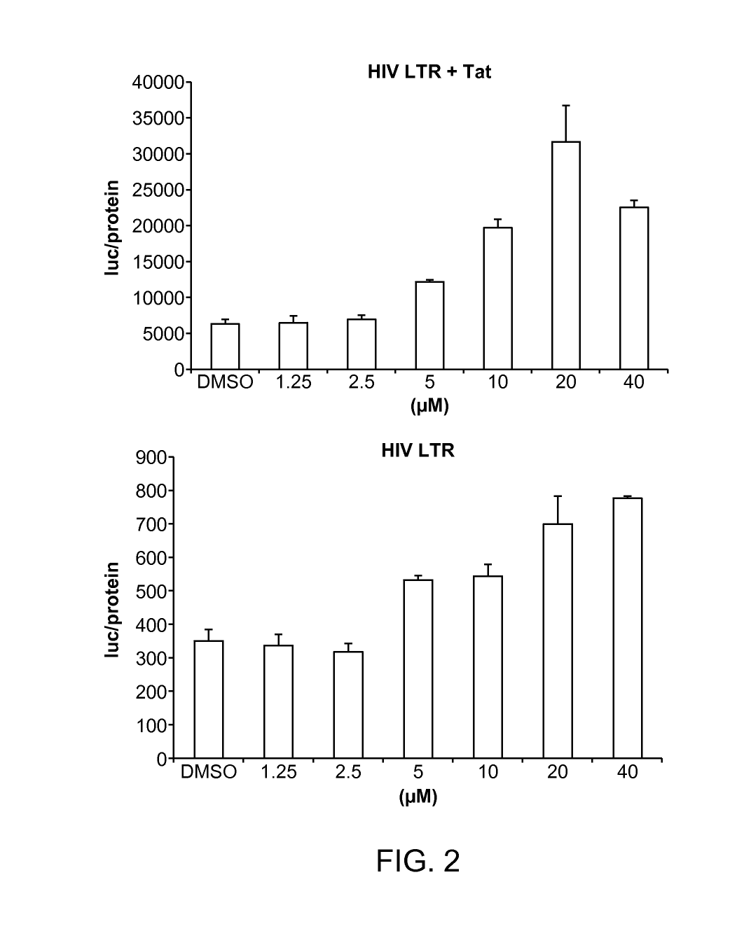

FIG. 2 depicts the effect of compound 6163501 on the activity of the HIV promoter in the presence (HIV LTR+Tat) and absence (HIV LTR) of Tat.

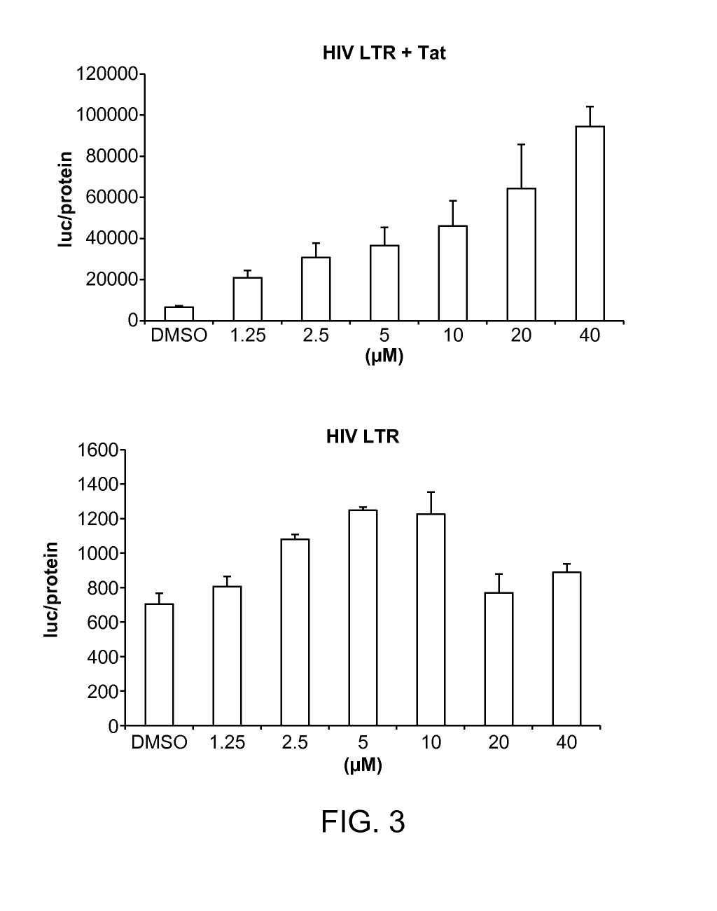

FIG. 3 depicts the effect of compound 791084 on the activity of the HIV promoter in the presence (HIV LTR+Tat) and absence (HIV LTR) of Tat.

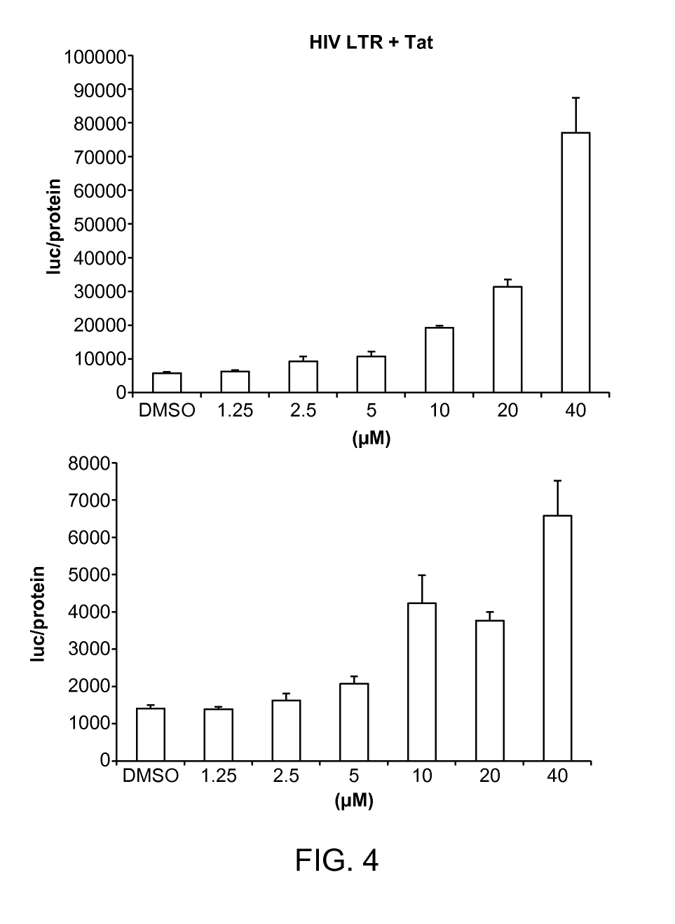

FIG. 4 depicts the effect of compound 7910896 on the activity of the HIV promoter in the presence (HIV LTR+Tat) and absence (HIV LTR) of Tat.

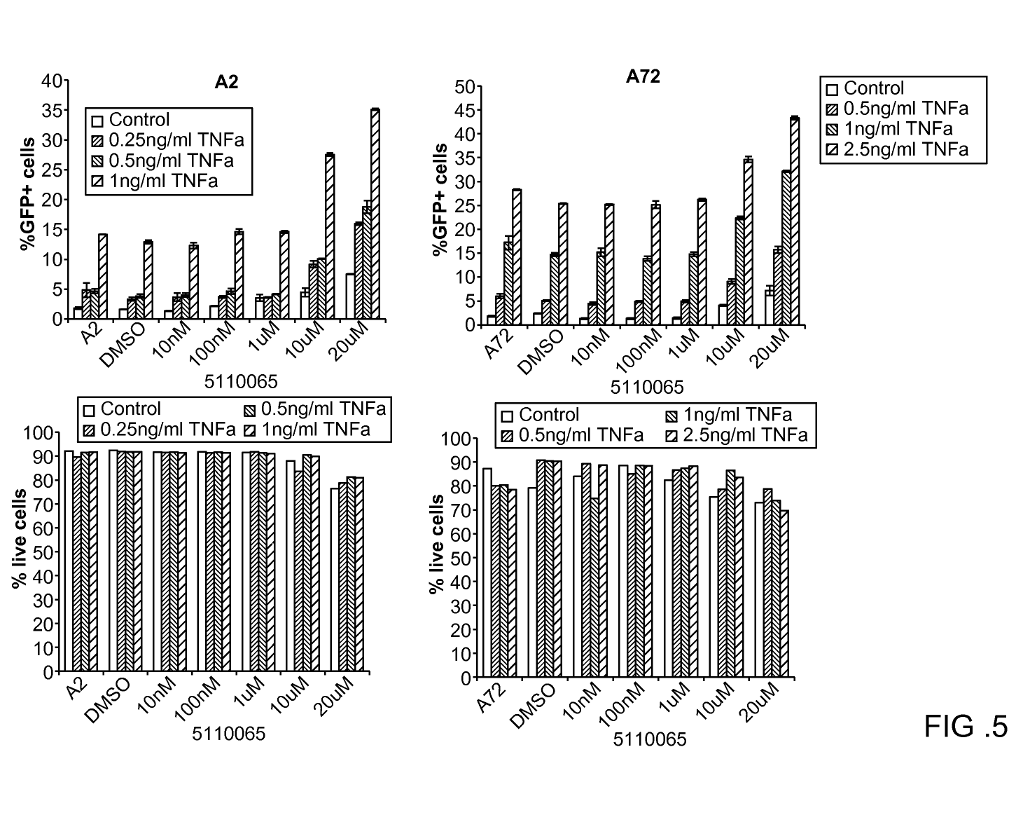

FIG. 5 depicts the effect of compound 5110065 on HIV expression as monitored by flow cytometry measurement (or FACS analysis) of GFP expressing cells. Comparative data is shown for A2 cells, (Jurkat cells contain a latent integrated LTR-Tat-IRES-GFP retroviral vector (Jordan et al. (2003) EMBO J. 22(8): 1868-1877)) and A72 cells (Jurkat cells containing an integrated LTR-GTP retroviral vector lacking Tat (Jordan et al. EMBO J. 2001, 20 1726-1738)). The concentration of compound was varied from 10 nM to 20 μM. The bottom panels depict the % live cells following treatment. The upper panels depict the % of those live cells that are GFP+. For the left panels, for each condition on the X axis, 4 bars are shown (from left to right: Control, 0.25 ng/ml TNFa (TNF-α), 0.5 ng/ml TNFa, and 1 ng/ml TNFa. For the right panels, for each condition on the X axis, 4 bars are shown (from left to right: Control, 0.5 ng/ml TNFa, 1 ng/ml TNFa, and 2.5 ng/ml TNFa.

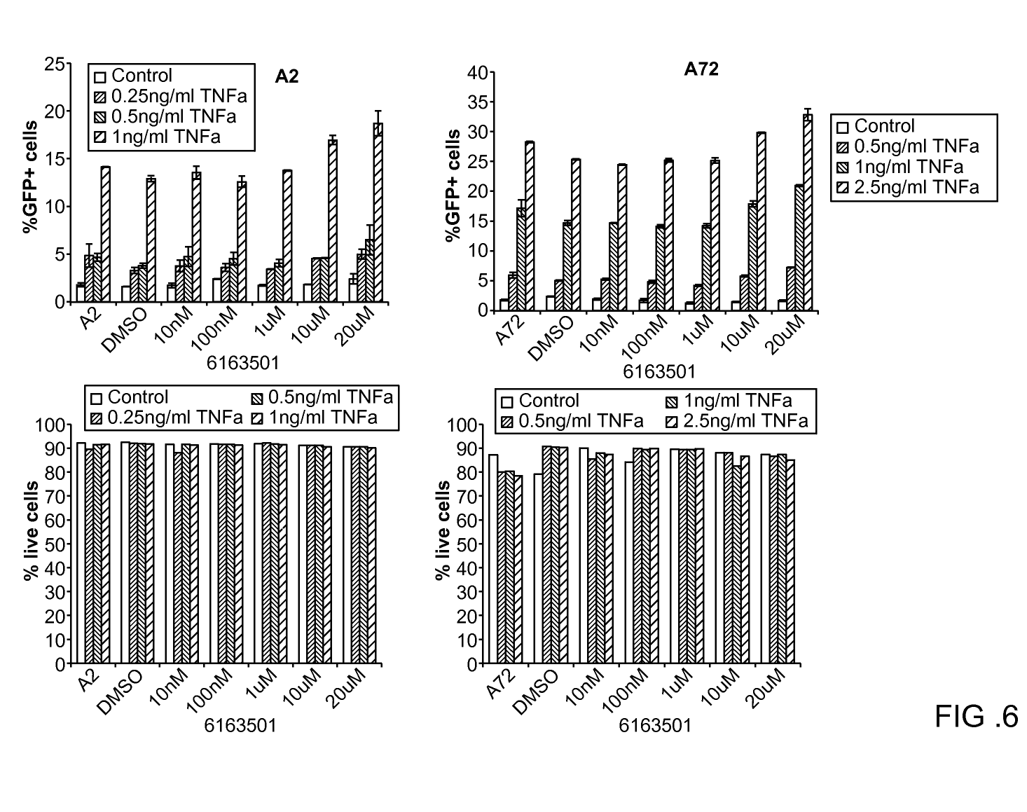

FIG. 6 depicts the effect of compound 6163501 on HIV expression as monitored by flow cytometry measurement of GFP expressing cells. Comparative data are shown for A2 and A72 cells. For the left panels, for each condition on the X axis, 4 bars are shown (from left to right: Control, 0.25 ng/ml TNFa, 0.5 ng/ml TNFa, and 1 ng/ml TNFa. For the right panels, for each condition on the X axis, 4 bars are shown (from left to right: Control, 0.5 ng/ml TNFa, 1 ng/ml TNFa, and 2.5 ng/ml TNFa.

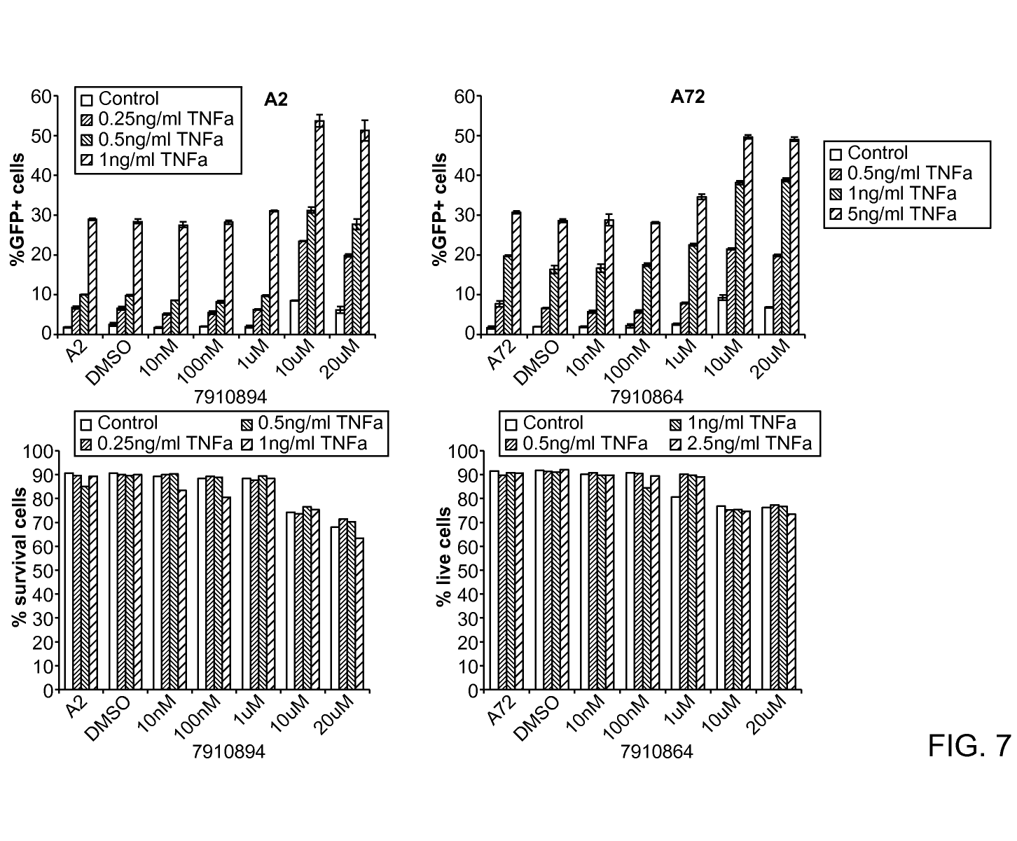

FIG. 7 depicts the effect of compound 7910894 on HIV expression as monitored by flow cytometry measurement of GFP expressing cells. Comparative data are shown for A2 and A72 cells. For the left panels, for each condition on the X axis, 4 bars are shown (from left to right: Control, 0.25 ng/ml TNFa, 0.5 ng/ml TNFa, and 1 ng/ml TNFa. For the upper right panel, for each condition on the X axis, 4 bars are shown (from left to right: Control, 0.5 ng/ml TNFa, 1 ng/ml TNFa, and 5 ng/ml TNFa. For the lower right panel, for each condition on the X axis, 4 bars are shown (from left to right: Control, 0.5 ng/ml TNFa, 1 ng/ml TNFa, and 2.5 ng/ml TNFa.

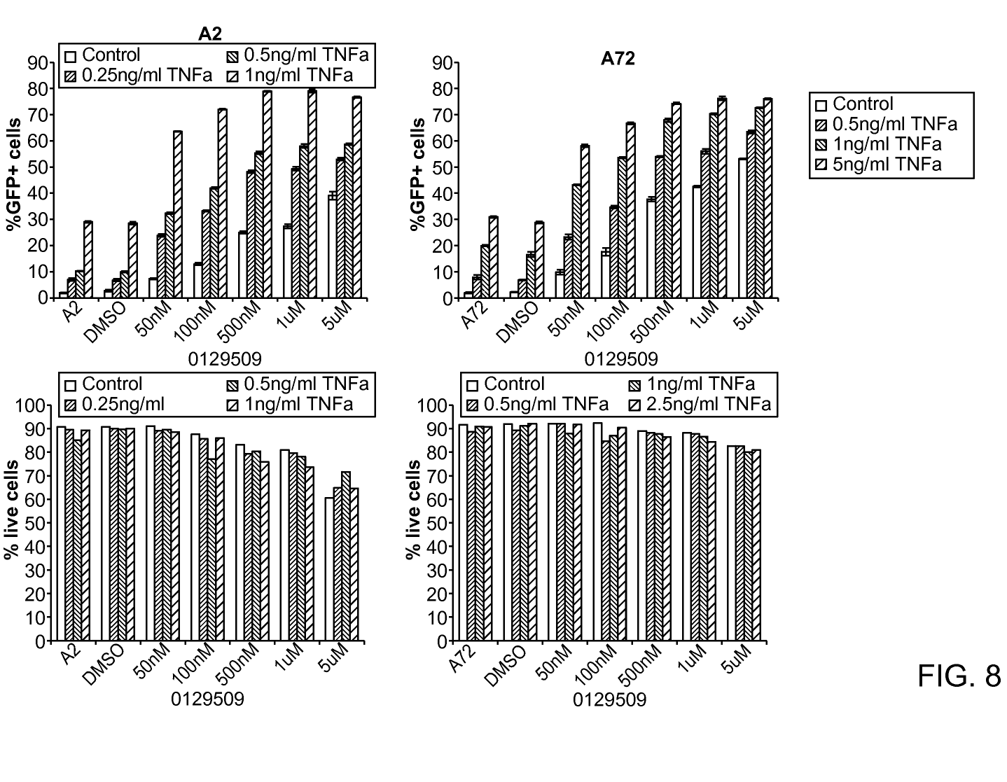

FIG. 8 depicts the effect of compound 129509 on HIV expression as monitored by flow cytometry measurement (of GFP expressing cells. Comparative data are shown for A2 and A72 cells. For the left panels, for each condition on the X axis, 4 bars are shown (from left to right: Control, 0.25 ng/ml TNFa, 0.5 ng/ml TNFa, and 1 ng/ml TNFa. For the upper right panel, for each condition on the X axis, 4 bars are shown (from left to right: Control, 0.5 ng/ml TNFa, 1 ng/ml TNFa, and 5 ng/ml TNFa. For the lower right panel, for each condition on the X axis, 4 bars are shown (from left to right: Control, 0.5 ng/ml TNFa, 1 ng/ml TNFa, and 2.5 ng/ml TNFa.

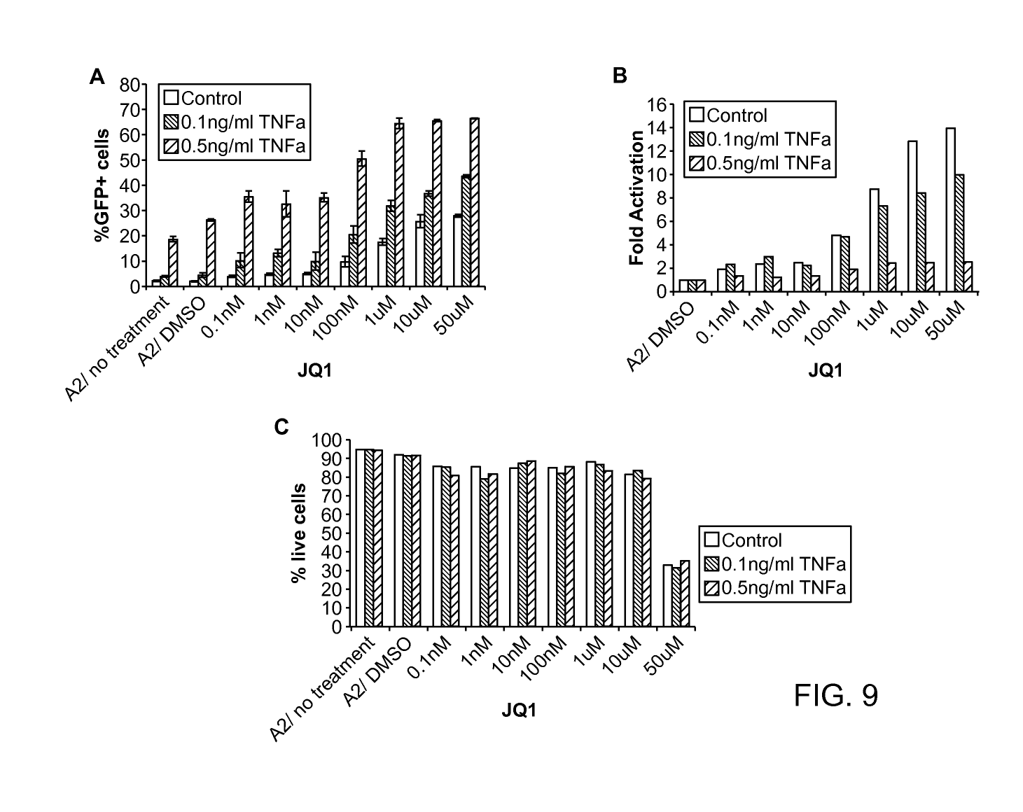

FIGS. 9A-C depict the effect of compound JQ1 on HIV expression in A2 cells as monitored by flow cytometry measurement of GFP expression. Panel (A) shows the % of live cells that are GFP+, Panel (B) demonstrates the level of HIV activation and Panel (C) indicates the % live cells. For all panels, for each condition on the X axis, 3 bars are shown (from left to right: Control, 0.1 ng/ml TNFa, 0.5 ng/ml TNFa.

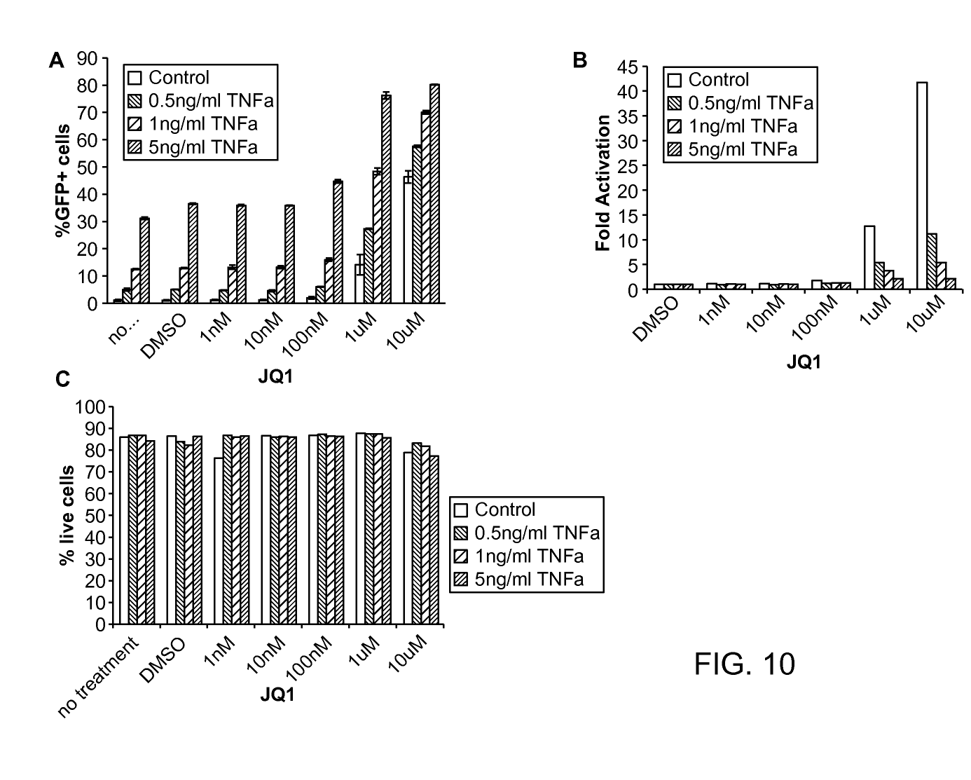

FIGS. 10A-C depict the effect of compound JQ1 on HIV expression in A72 cells as monitored by flow cytometry measurement of GFP expression. Panel (A) shows the % of live cells that are GFP+, Panel (B) demonstrates the level of HIV activation and Panel (C) indicates the % live cells. For all panels, for each condition on the X axis, 4 bars are shown (from left to right: Control, 0.5 ng/ml TNFa, 1 ng/ml TNFa, and 5 ng/ml TNFa.

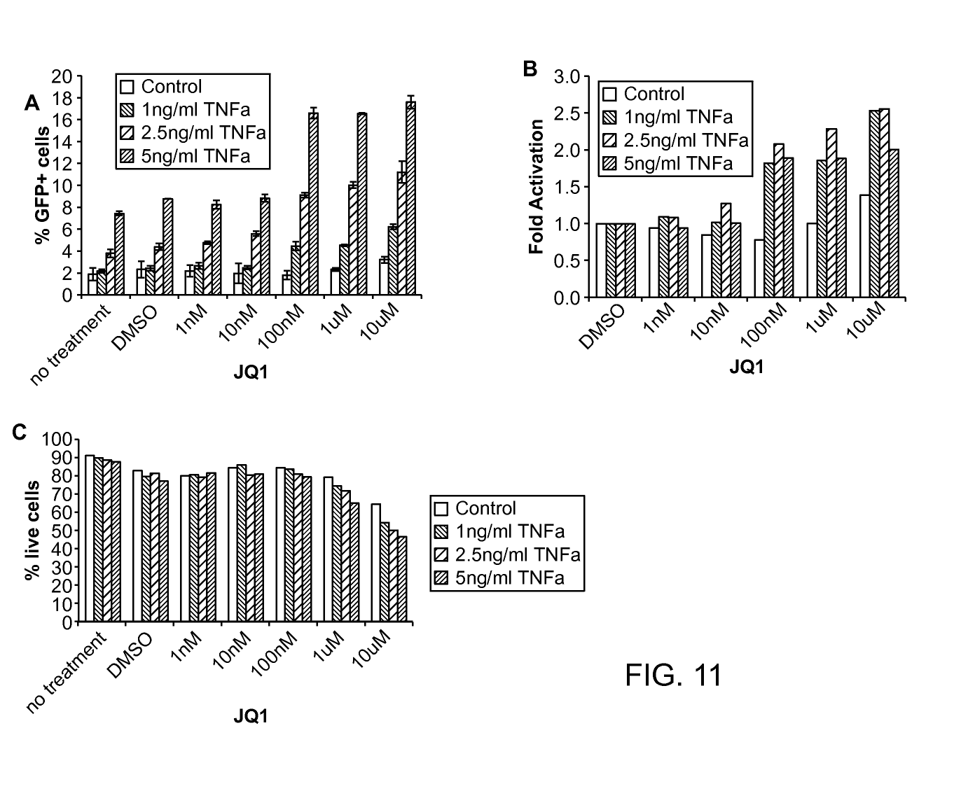

FIGS. 11A-C depict the effect of compound JQ1 on HIV expression in J-Lat 6.3 cells as monitored by flow cytometry measurement of GFP expression. Panel (A) shows the % of live cells that are GFP+, Panel (B) demonstrates the level of HIV activation and Panel (C) indicates the % live cells. For all panels, for each condition on the X axis, 4 bars are shown (from left to right: Control, 1 ng/ml TNFa, 2.5 ng/ml TNFa, and 5 ng/ml TNFa.

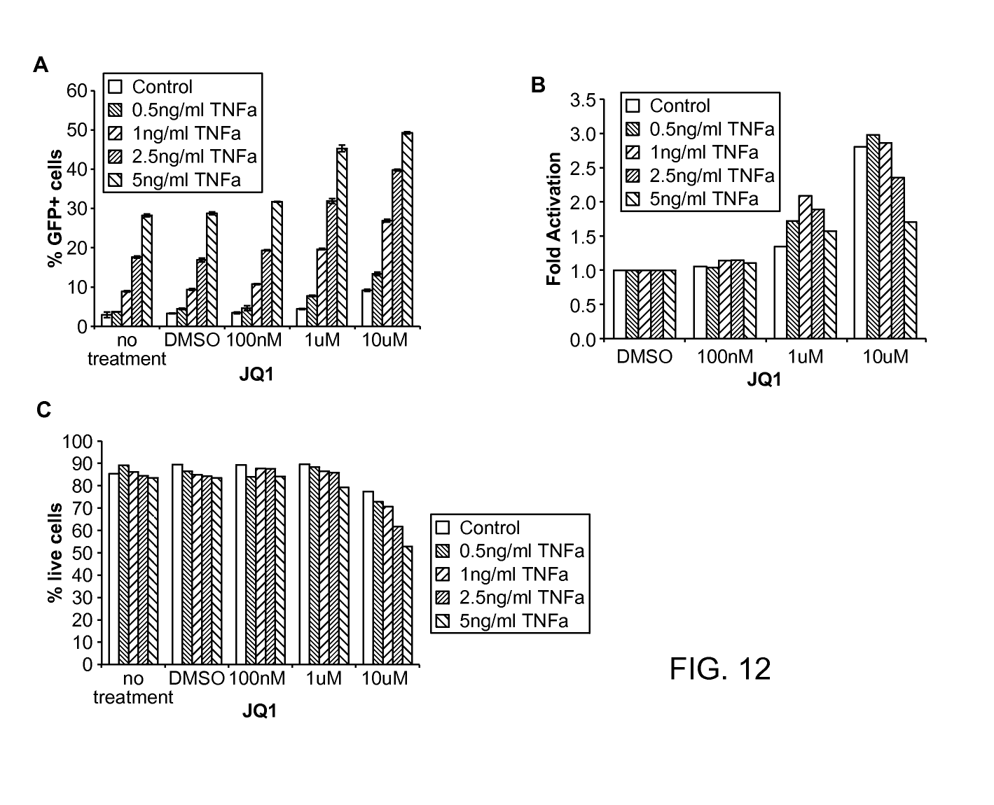

FIGS. 12A-C depict the effect of compound JQ1 on HIV expression in J-Lat 11.1 cells as monitored by flow cytometry measurement of GFP expression. Panel (A) shows the % of live cells that are GFP+, Panel (B) demonstrates the level of HIV activation and Panel (C) indicates the % live cells. For all panels, for each condition on the X axis, 5 bars are shown (from left to right: Control, 0.5 ng/ml TNFa, 1 ng/ml TNFa, 2.5 ng/ml TNFa, and 5 ng/ml TNFa.

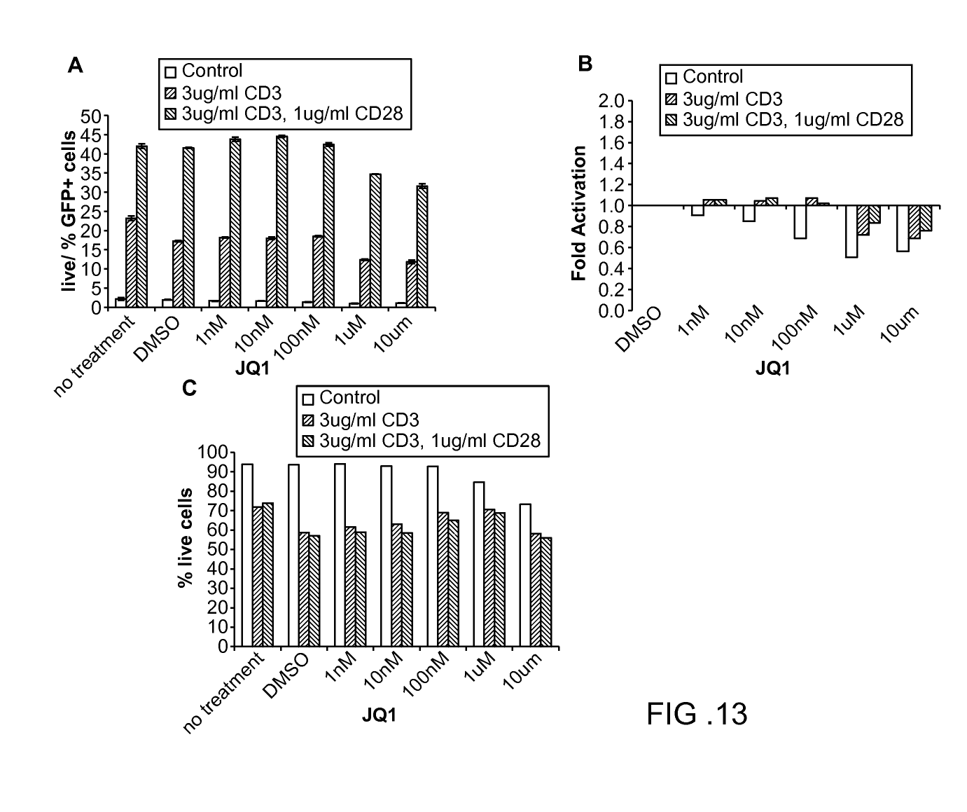

FIGS. 13A-C depict the effect of compound JQ1 on HIV expression in J-Lat 5A8 cells as monitored by flow cytometry measurement of GFP expression. Panel (A) shows the % of live cells that are GFP+, Panel (B) demonstrates the level of HIV activation and Panel (C) indicates the % live cells. The 5A8 cells are responsive to CD3/CD28 activation and not TNF-alpha. For all panels, for each condition on the X axis, 3 bars are shown (from left to right: Control; 3 μg/ml CD3; and 3 μg/ml CD3 with 1 μg/ml CD28.

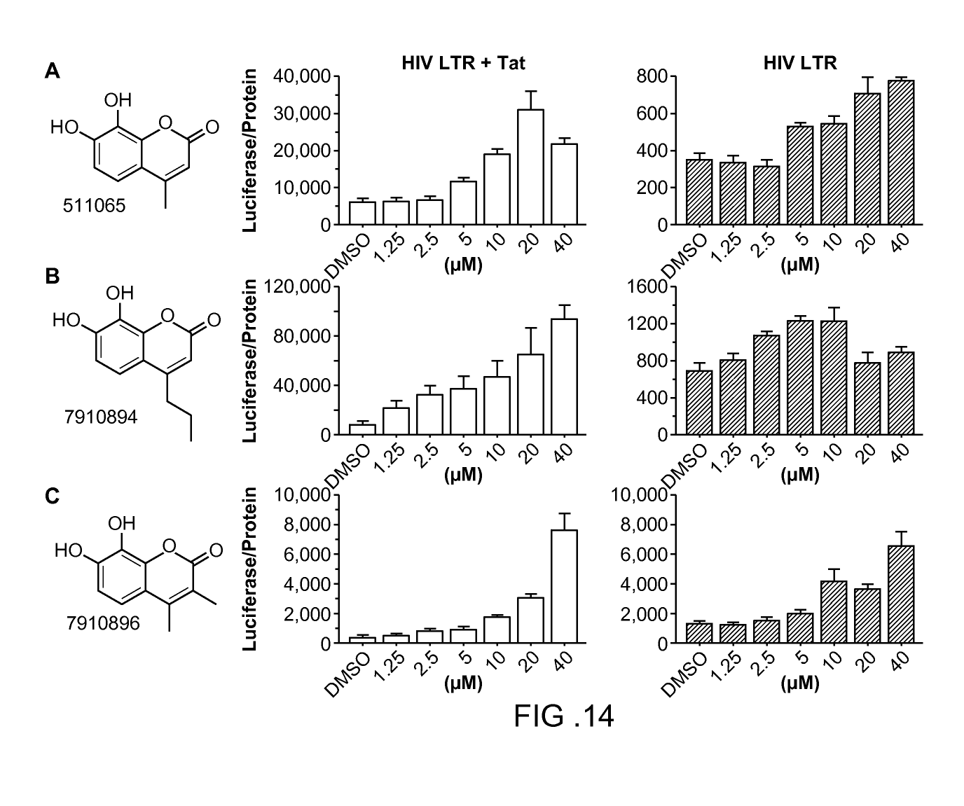

FIGS. 14A-C depict the effect of compounds 5110065, 7910894, and 7910896 on the activity of the HIV promoter in the presence (HIV LTR+Tat) and absence (HIV LTR) of Tat.

FIG. 15 depicts the effect of compound JQ1 on HIV expression in A2 and A72 cells. For the Left panels, for each condition on the X axis, 3 bars are shown (from left to right: Control; 0.1 ng/ml TNFα, and 0.5 ng/ml TNFα. For the Right panels, for each condition on the X axis, 4 bars are shown (from left to right: Control; 0.5 ng/ml TNFα, 1 ng/ml TNFα, and 5 ng/ml TNFα.

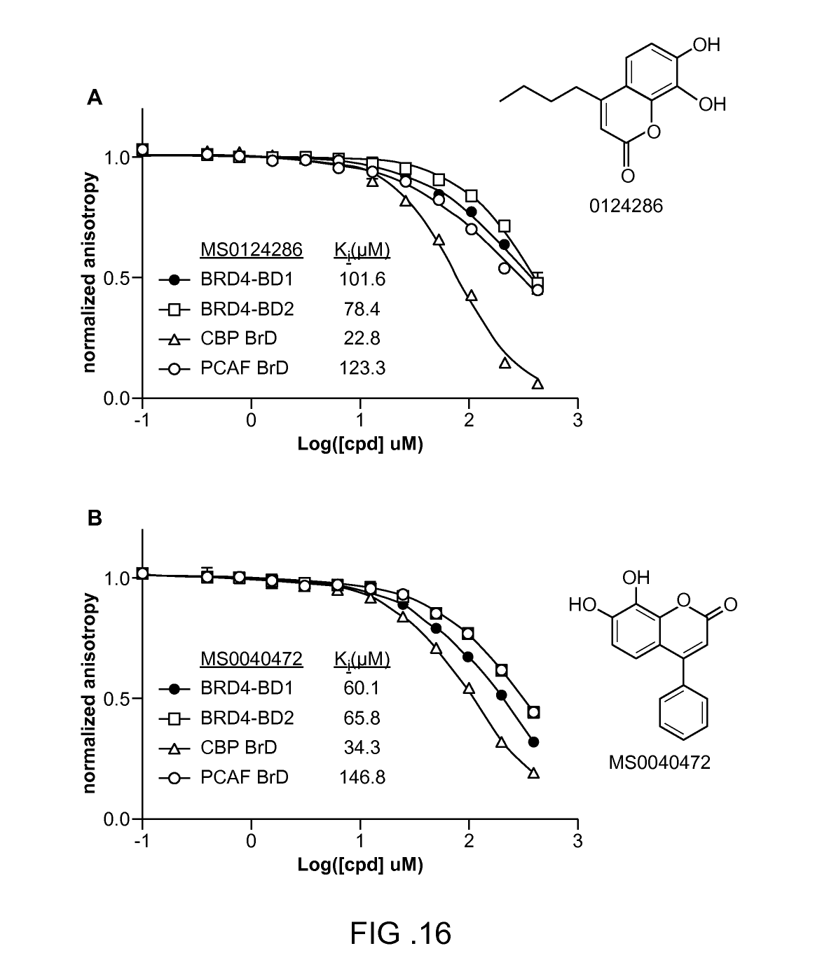

FIGS. 16A and 16B depict the binding affinity measurements of MS0124286 and MS0040472 to the bromodomains from the human proteins BRD4, CBP, and PCAF, as determined in a fluorescence anisotropy competition assay using a FITC-labelled MS417 as an assay probe.

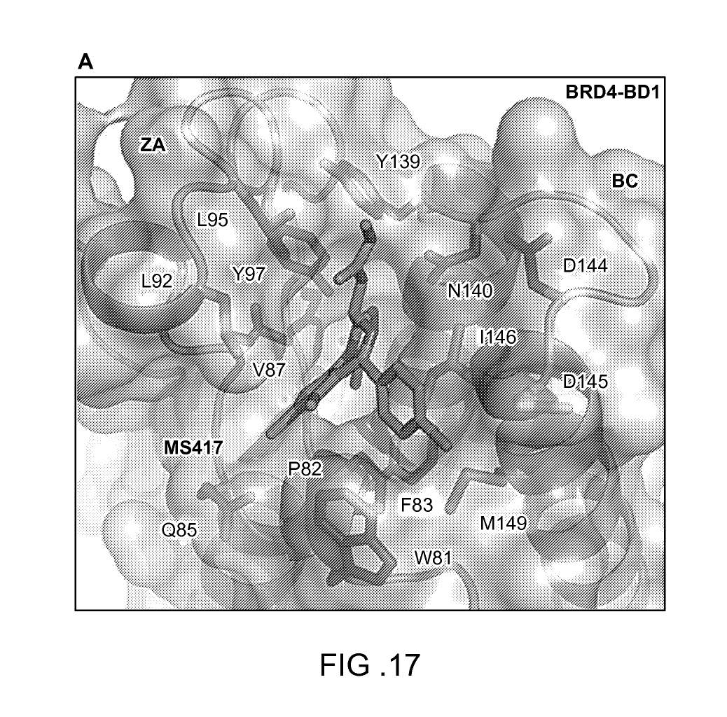

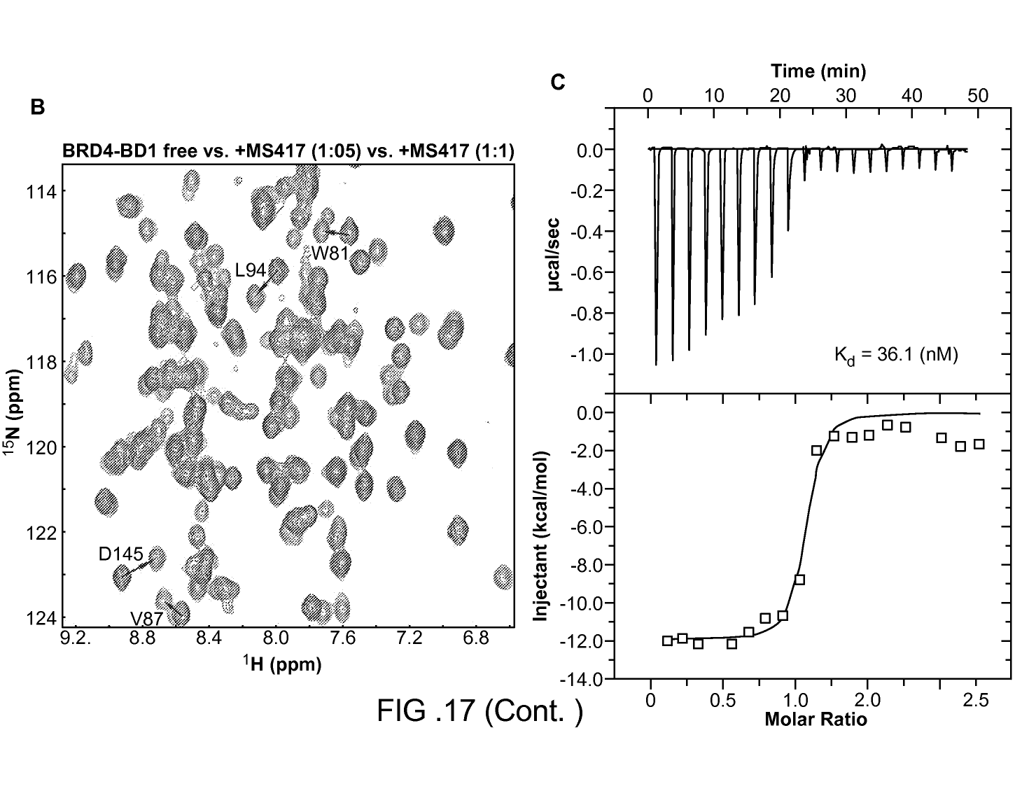

FIGS. 17A-C depict features of the BRD4 bromodomain inhibitor MS147. (A) X-ray crystal structure of MS417 bound to BRD4-BD1. (B) 2D 1H-15N HSQC spectra of BRD4-BD1 in the free form and in the presence of the BrDi MS417 with protein:ligand molar ratio of 1:0.5 and 1:1. (C) Isothermal titration calorimetry (ITC) measurement of BRD4-BD1 binding MS417.

FIG. 18 depicts the chemical structures of various exemplary BRD inhibitors.

### Example 2

### BET Bromodomain-Targeting Compounds Reactivate HIV from Latency Via a Tat-Independent Mechanism

### Materials and Methods

HEK293T and Jurkat cells were obtained from the American Type Culture Collection. J-Lat (clones A2 and A72) cell lines were described. HEK293T cells were cultured in DMEM supplemented with 10% FBS, 1% L-glutamine and 1% penicillin-streptomycin (Life Technologies). Jurkat and J-Lat cells were cultured in RPMI supplemented with 10% FBS, 1% L-glutamine, and 1% penicillin-streptomycin. TNFα (Sigma-Aldrich) was used at concentrations of 0.25 or 1 ng/ml. Human αCD3 and αCD28 (BD Biosciences) were used at concentrations of 3 and 1 μg/ml, respectively. Prostratin (Sigma) was used at a suboptimal concentration of 0.5 μM, and SAHA (NCI Chemical Carcinogen Repository, Midwest Research Institute) was used at 2.5 μM.

### Primary T-Cell Model of HIV Latency in Bcl2-Transduced T Cells

Primary CD4⁺ T cells from healthy donors were isolated to generate HIV-1 latent infection in vitro as described (Yang et al. J Clin Invest 2009; 119:3473-86). In brief, primary CD4⁺ T cells were costimulated with αCD3 and αCD28 antibodies and then transduced with a Bcl-2-expressing lentiviral vector to allow long-term culture. After 3 weeks of culture in absence of supplemental cytokines, Bcl-2-transduced primary CD4⁺ T cells return to a quiescent state. Bcl-2-transduced cells were then costimulated and infected with NL4-3-Δ6-drEGFP virus. The infected cells were cultured for several weeks without supplemental cytokines to allow establishment of latency in surviving cells. Flow cytometric cell sorting was used to remove residual GFP⁺ cells. This approach produces cultures, in which 1-3% of the cells are latently infected, with the remaining cells (>97%) being uninfected. After cell sorting, the purified GFP⁻ bcl-2-transduced resting CD4⁺ T cells, including latently infected cells, were plated at 5×10⁴ cells/well, in 200 μL of RPMI 1640+10% FBS in U-bottomed 96-well plates, and treated with the indicated compounds for 24-72 h at 3TC. Cells treated with 2.5 μg/ml αCD3 plus 1 μg/ml αCD28 antibodies were used as positive controls. At the indicated times, the fraction of GFP⁺CD4⁺ T cells was measured by FACS.

### ShRNA-Mediated Knockdown Experiments and Flow Cytometry Analysis

ShRNA-expressing lentiviral vectors were purchased from Open Biosystems. The plasmids TRCN0000006308 and TRCN0000006310 were used to deplete BRD2, plasmids TRCN0000021424 and TRCN0000021428 were used to deplete BRD4, and plasmids TRCN0000013673 and TRCN0000013675 were used to deplete cyclin T1. The pLKO.1 vector containing scramble shRNA was used as control. Pseudotyped viral stocks were produced in 2×10⁶ HEK293T cells by the calcium phosphate method by cotransfection 10 μg of shRNA-expressing lentiviral vectors, together with 6.5 μg of the lentiviral packaging construct pCMVdelta R8.91 and 3.5 μg of VSV-G glycoprotein-expressing vector, and titered for p24 content. J-Lat A72 cells (containing a LTR-GFP construct) were spininfected with virus (1 ng of p24 per 10⁶ cells) containing shRNAs against BRD2, BRD4, cyclin T1 or nontargeting control shRNAs and were selected with puromycin (2 μg/ml; Sigma) (Naldini L, et al., Science 1996; 272:263-7). After 4 days of selection, cells were treated with the indicated concentration of drugs. The percentage of GFP⁺ cells was determined after 18 h using a MACSQuant VYB FACS analyzer (Miltenyi Biotech GmbH). Cell viability was monitored by forward and side scatter analysis. Analysis was conducted on 3×10,000 live cells per condition, and all experiments were independently repeated at least three times. Data were analyzed using FlowJo 9.4 (Tree Star).

### Single-Cell Analysis of JQ1-Treated Cells

Lentiviral vectors expressing the LTR-GFP cassette in the absence of Tat were used to infect 5×10⁵ Jurkat cells at a multiplicity of infection <0.1, resulting in 25,000-50,000 infected cells each presumably with a unique integration site. Cells were then sorted by FACS to isolate green fluorescent protein-labeled (GFP⁺) cells and fluorescently imaged on glass-bottom dishes in RPMI 1640 with 10% fetal calf serum and 1% penicillin-streptomycin and 1 μM JQ1 at t=0 h for the treated population. The imaging took place in humidified conditions at 37° C. and 5% CO₂ for 12-24 hours with a 40× (1.2 NA) oil-immersion objective on a Zeiss Observer Z1 microscope equipped with an automated linear-encoded X-Y stage. Image processing and cell tracking were performed in Matlab™ with an in-house algorithm (Weinberger et al. Nature Genet 2008; 40:466-70) and a single 12-hour experiment could generate up to 1000 single-cell trajectories for analysis.

For each trajectory, noise autocorrelation (Φ(t)) and magnitude (CV2) were calculated using an established noise processing algorithm (Weinberger et al. Nature Genet 2008; 40:466-70; Austin D W et al., Nature 2006; 439:608-11) A published theory (Simpson M L, et al. J Theor Biol 2004; 229:383-94; Cox C D, et al. Proc Natl Acad Sci USA 2008; 105:10809-14) of the two-state transcriptional bursting model yields analytical expressions for the autocorrelation of the noise, Φ(τ), and noise magnitude. Derivations and calculated burst size and frequency have been reported (Singh A, et al. Biophysical Journal 2010; 98:L32-L4). Exogenous JQ1 addition can change either mean fluorescence, variability of expression (defined by the coefficient of variation, CV), or both. Modeling of 2-state transcription enables the differentiation between modulations in transcriptional initiation (Dkₒₙ) or in the burst size (DT/k_{off}).

### Results

### JQ1 Activates HIV Transcription in a Tat-Independent Manner

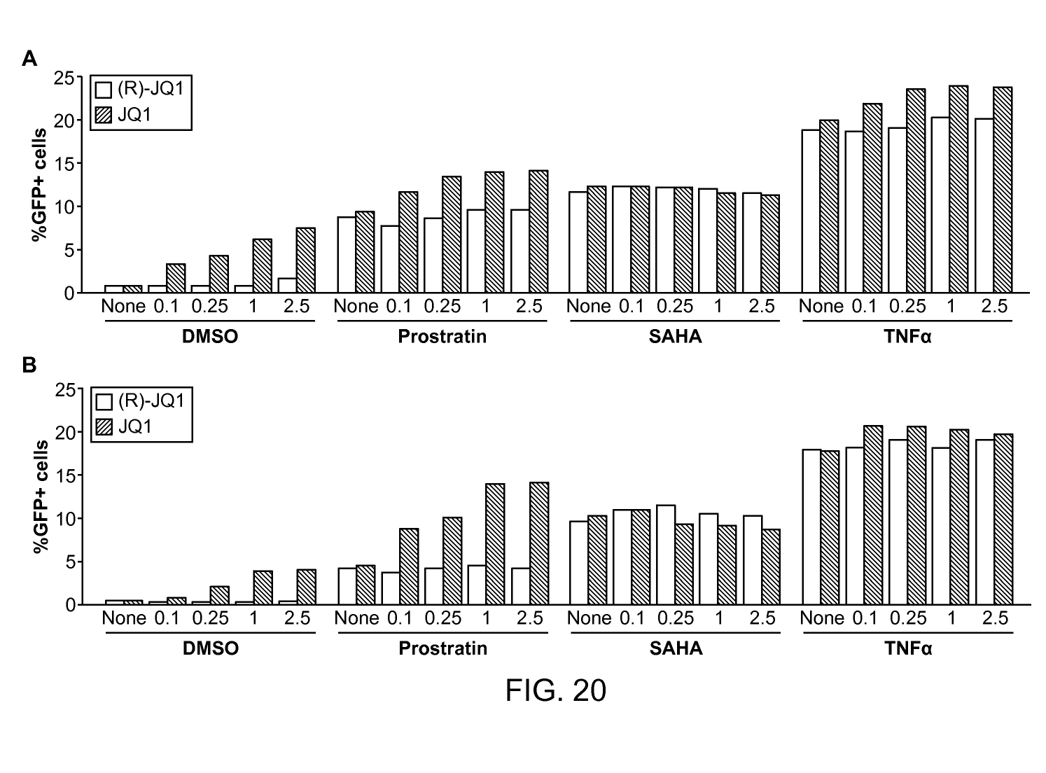

A polyclonal population of Jurkat T cells containing latent HIV (clone R7/E-/GFP) was treated with increasing amounts of JQ1. This viral clone contains a frameshift mutation in the viral Env gene to prevent viral spread and expresses GFP in the Nef open reading frame, which allows separation of actively infected GFP⁺ from GFP⁻ cells by cell sorting. GFP⁻ cells, which are mostly uninfected but contain a small fraction of latently infected cells with silenced HIV transcription, were treated with JQ1. Activation of transcription was measured by flow cytometry of GFP. JQ1, but not the stereoisomer control (R)-JQ1, reactivated HIV-1 in a dose-dependent manner (FIG. 20A). Stimulation of cells with JQ1 produced up to fivefold more GFP-expressing cells than control-treated cells. Similar results were obtained with another viral clone (NL4-3/E-/GFP-IRES-nef), which also expresses GFP in the Nef position and also has Nef expressed under the control of an IRES element (FIG. 20B).

Next, JQ1 reactivation was tested in combination with HDAC inhibitor suberoylanilidehydroxamic acid (SAHA), the protein kinase C (PKC) activator prostratin or the proinflammatory cytokine TNFα. Enhanced activation resulted when JQ1 was added with prostratin, while no additive or synergistic effects were observed with SAHA (FIG. 20A, B). Co-treatment with TNFα led to a very modest enhancement of the JQ1 effect in this system (FIG. 20A, B).

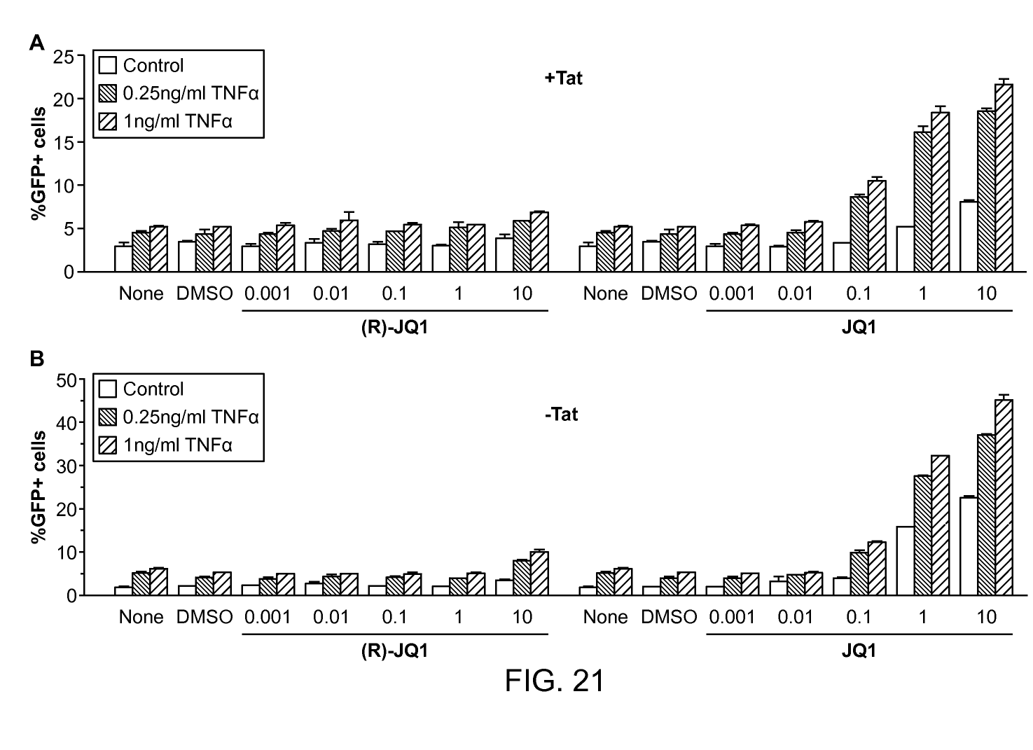

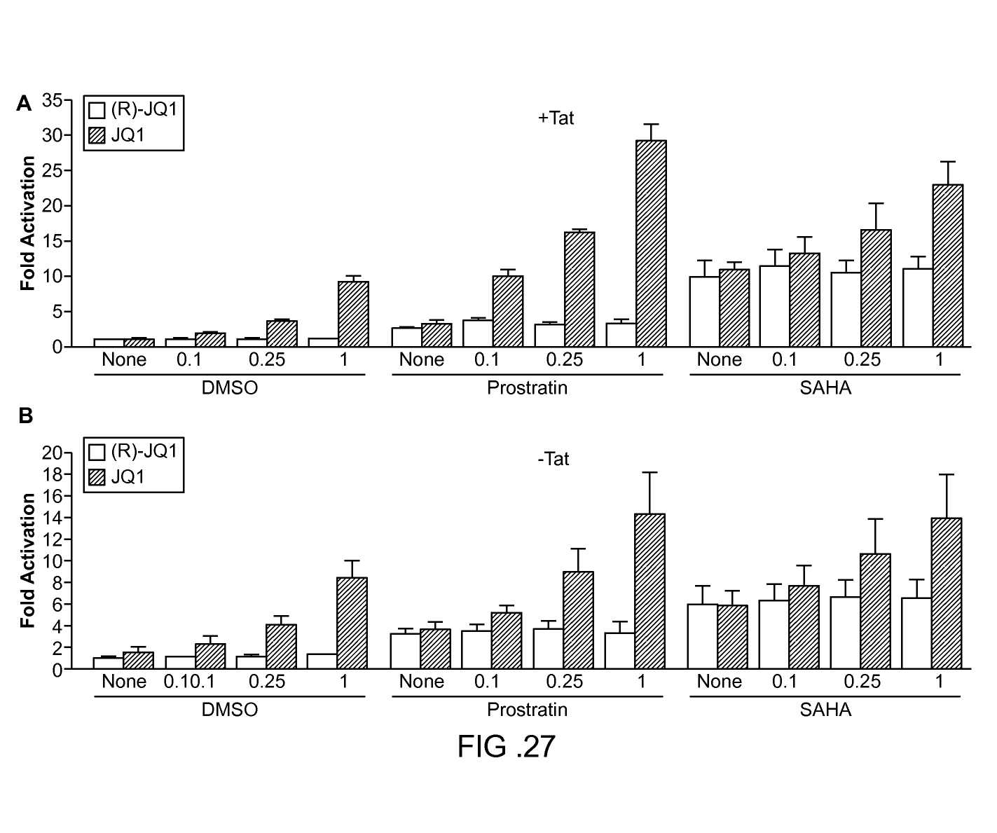

To determine if BET inhibition specifically activates Tat-dependent transcription, a J-Lat cell line was utilized that harbored a latent lentiviral construct expressing Tat with GFP from the HIV LTR (clone A2; LTR-Tat-IRES-GFP). Treatment with JQ1, but not inactive (R)-JQ1, activated HIV transcription in a dose-dependent manner as measured by flow cytometry of GFP (FIG. 21A). Stimulation with JQ1 yielded up to ninefold more GFP-expressing cells than control-treated cells, and a 22-fold increase was observed when cells were co-treated with JQ1 and low doses of TNFα (FIG. 21A). However, this effect was not specific for Tat: the same effect was observed in A72 cells, containing a latent LTR-GFP construct lacking Tat. Here, an up to 22-fold increase in GFP⁺ cells resulted from JQ1 treatment alone and a 45-fold increase when TNFα was added with JQ1 (FIG. 21B). Both cell lines were also treated with prostratin and SAHA (FIG. 27). As observed with the polyclonal cell populations, adding prostratin to JQ1 enhanced the JQ1 effect, while only a very modest increase was observed with SAHA, indicating that SAHA and JQ1 target a similar cellular pathway. Collectively, these results establish the effectiveness of JQ1 to reverse HIV latency in a Tat-independent manner.

### Activating Potential of Known BET Inhibitors in Cell Lines and a Primary T-Cell Model of HIV Latency

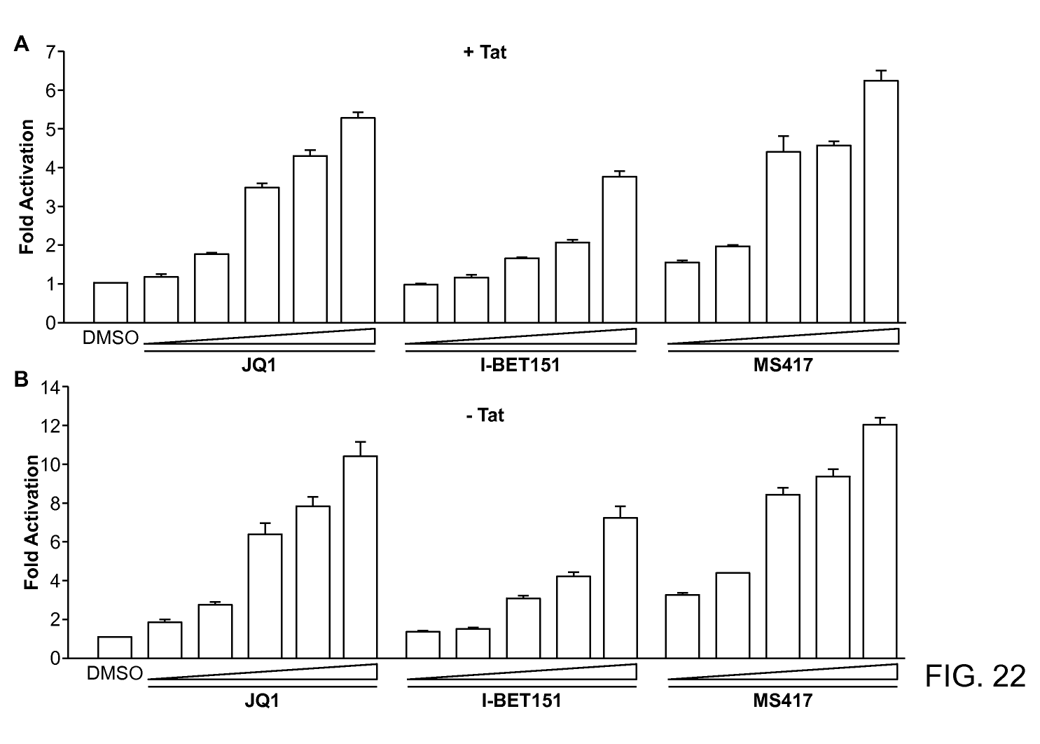

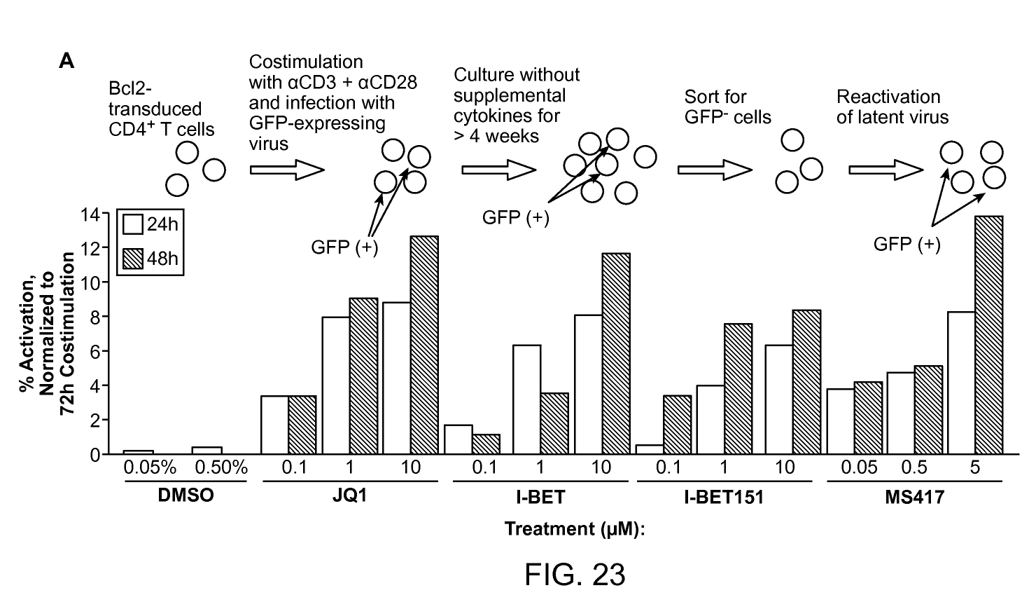

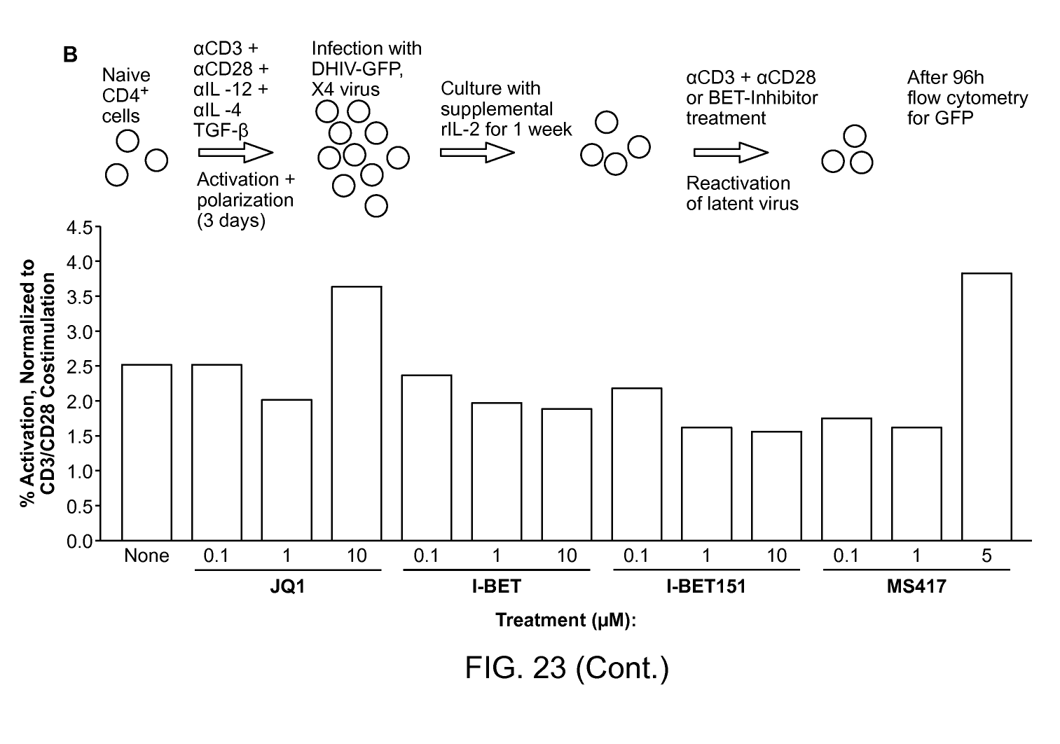

The activating effect was not unique to JQ1 but was also observed with I-Bet151 and MS417, two recently reported small-molecule bromodomain inhibitors with similar binding affinities to BET proteins (FIG. 22A, B). Both compounds effectively activated HIV from latency in A2 and A72 cell lines, underscoring the notion that the BET inhibitor effect on HIV latency is independent of Tat. These compounds were also tested in a primary T-cell model of latency. In this model, Bcl-2-transduced CD4⁺ T cells were infected in a single-round infection with HIV clone NL4-3-Δnef-Δpol-EGFP to generate robust latent infection in vitro (FIG. 23A). To reactivate latent HIV-1, cells were treated with the indicated compounds or a combination of αCD3 and αCD28 antibodies as a control for maximal activation. JQ1 reactivated latent HIV-1 at ˜14% of the rate achieved by costimulation with αCD3 and αCD28 antibodies (FIG. 23A). The same activation was observed in cells activated with I-Bet, I-Bet151, and MS417, supporting the model that BET inhibition has the potential to reverse latency in primary T cells. However, when a second primary T-cell model of latency was tested using ex vivo differentiated nonpolarized CD4⁺ T cells, there was no significant reversal of latency with any of the BET inhibitor compounds with only a minor activatory effect observed with the highest doses of JQ1 and MS417 (FIG. 23B). Notably, current primary T-cell models of latency are diverse, and it is unknown which one faithfully reproduces the in vivo situation of latently infected cells. Interestingly, the nonpolarized T helper cell model of HIV latency is also resistant to reactivation by SAHA, underlining that BET inhibitors and SAHA may target common mechanistic pathways.

### Involvement of P-TEFb in the JQ1 Effect on HIV Latency

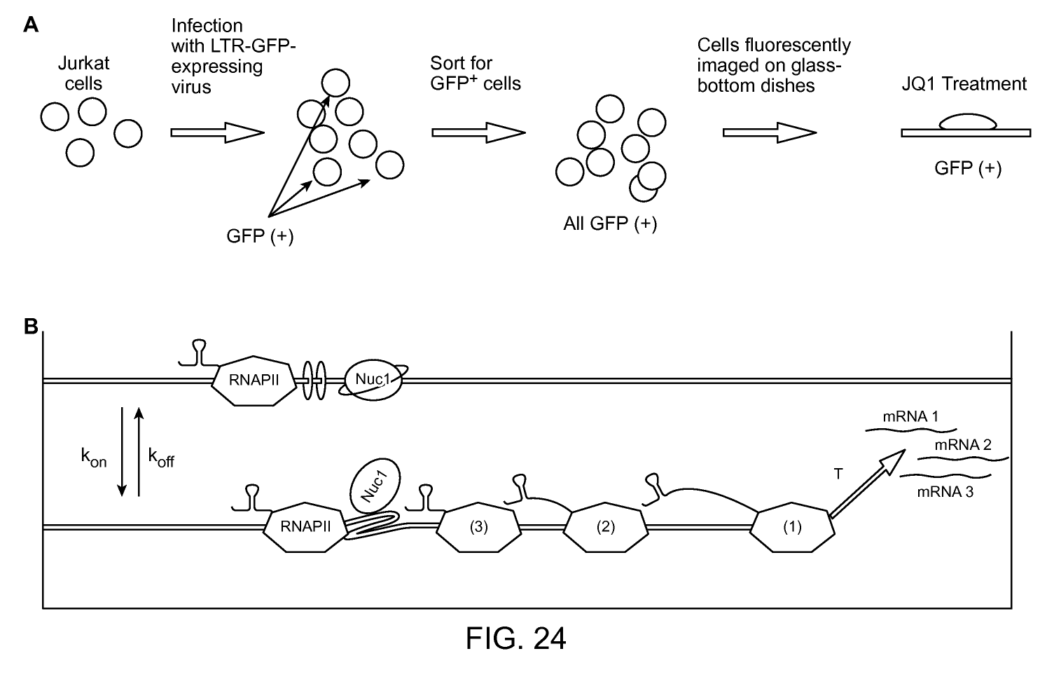

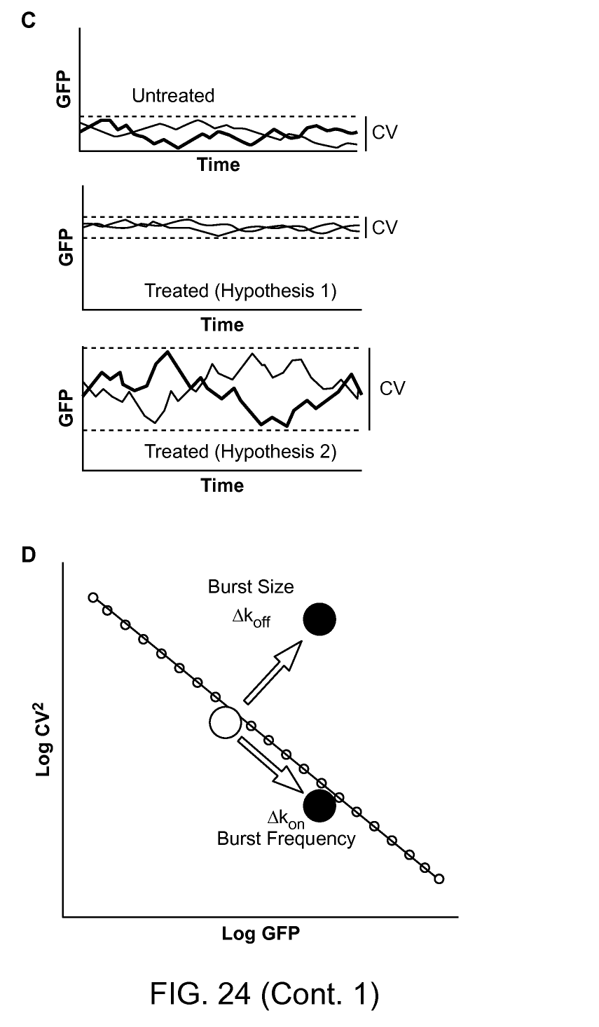

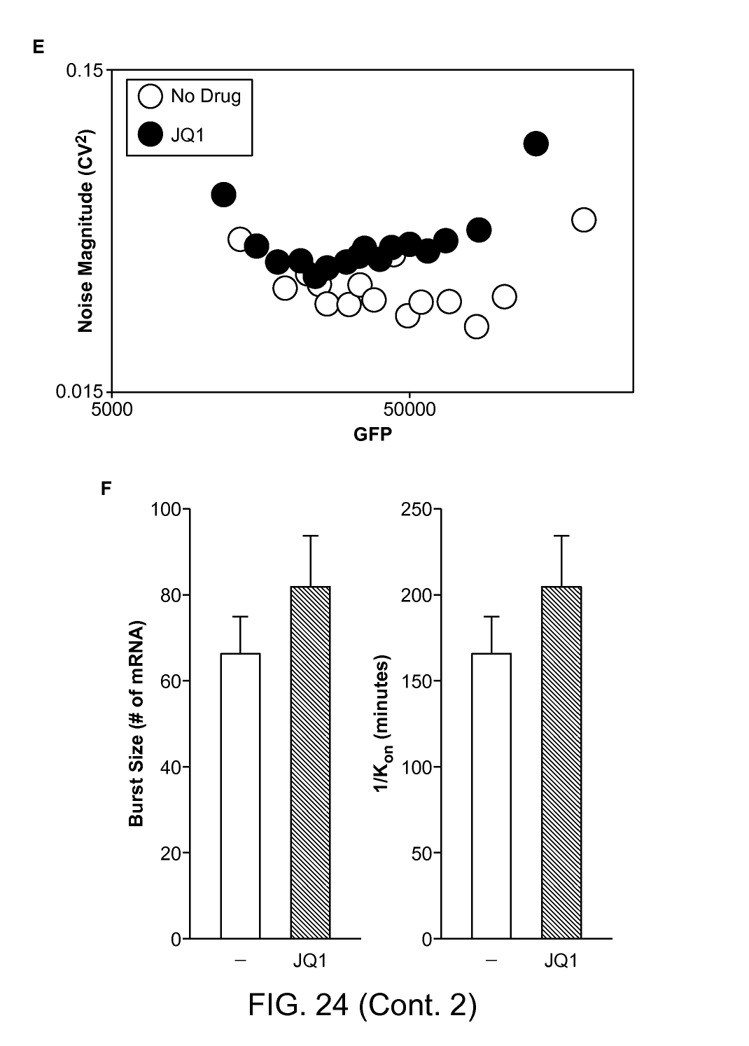

To biophysically characterize the Tat-independent JQ1 effect on the HIV LTR, single-cell time-lapse fluorescence microscopy was performed on ˜2,000 Jurkat cells where each cell carried a different integration site of the LTR promoter driving a destabilized GFP reporter (FIG. 24A). Mean fluorescence intensity (MFI) and the magnitude of intensity fluctuations (i.e. the ‘noise’) were quantified for sub-clusters of polyclonal cells in response to JQ1 treatment (FIG. 24). The data was analyzed using an established two-state model of episodic transcription in which the LTR switches between a transcriptional OFF state, where RNA polymerase II is stalled, and a transcriptional ON state where elongation occurs and multiple mRNA transcripts are produced at a rate=T (FIG. 24B). In this model, promoter switching occurs with rates kₒₙ and k_{off} which generates pulses or bursts of transcription. Previous measurements show that the LTR typically exhibits k_{off}>>kₒₙ, such that large bursts in expression are punctuated by long dwell times in the OFF state.

To determine how JQ1 influences these biophysical parameters (k_{off}, kₒₙ, T), changes in LTR MFI and noise (measured by the coefficient of variation, CV, which is defined as the standard deviation over the mean) were examined and compared to the untreated LTR (FIG. 24C, D). By qualifying MFI and CV in time-lapse trajectories, changes in both burst frequency (kₒₙ) and burst size, or the # of mRNA produced per activity pulse, (T/k_{off}) can be determined.

The results show that, across thousands of integration sites, JQ1 treatment increases LTR noise without a significant shift in MFI (FIG. 24E). It is important to note that the lack of change in MFI change is only relative to cells already expressing high levels of GFP. Quantitative analysis of the increase in variability shows a JQ1-specific enhancement in transcription burst size (T/k_{off}) that occurs in parallel with an increase in the average dwell time in the OFF state (l/kₒₙ) (FIG. 24F). On average, these JQ1-induced changes are equivalent to ˜50-minute delays in transcriptional initiation coupled to a concomitant increase of ˜15 mRNAs per pulse of transcriptional activity. These results indicate that JQ1 enhances transcription elongation from the LTR, in the absence of Tat, while delaying re-initiation of the polymerase complex.

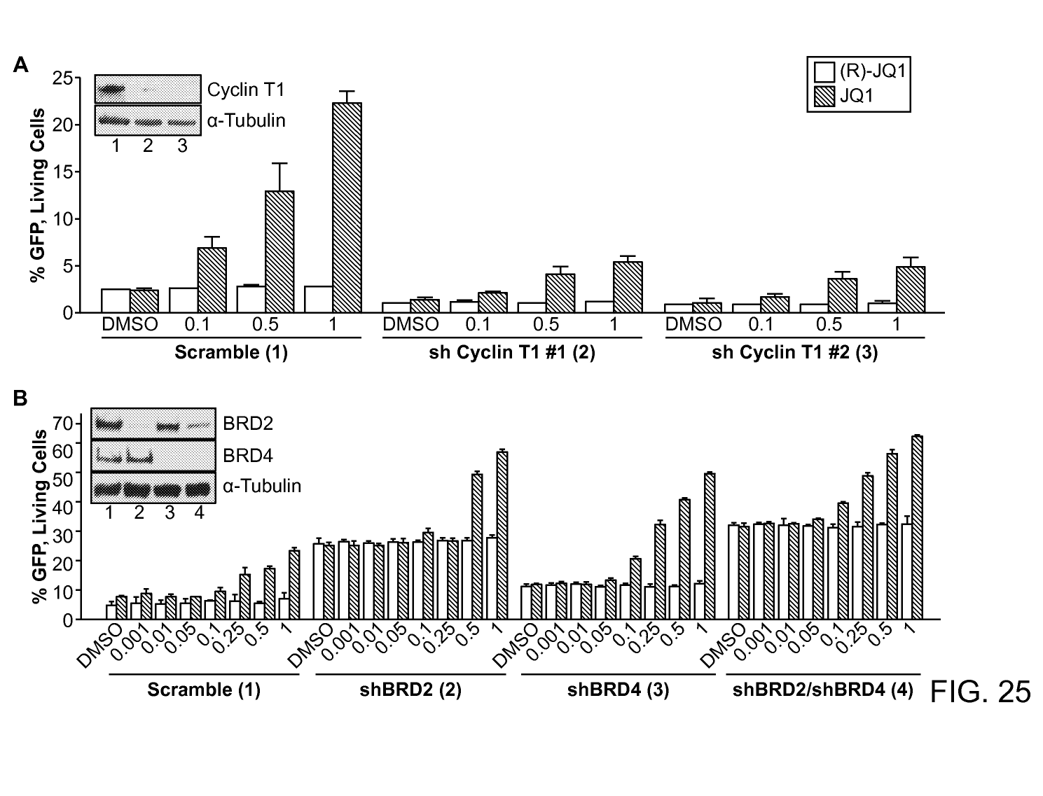

As transcription elongation at the HIV promoter uniquely depends on P-TEFb, whether the JQ1 effect in latent cells requires intact P-TEFb was determined. Short hairpin RNAs (shRNAs) directed against cyclin T1, an important component of P-TEFb, were introduced into A72 cells with lentiviral vectors. Knockdown of cyclin T1 yielded lower basal levels in GFP⁺ cells than control cells expressing nontargeting shRNAs (FIG. 25A). Importantly, cyclin T1 knockdown also decreased the ability of the cells to respond to JQ1 treatment. Only half as many cells responded with GFP expression when JQ1 was added than in control cells (FIG. 25A). Similar results were observed with two independent cyclin T1-targeting shRNAs, confirming that P-TEFb is involved in the reactivating effect of JQ1 in the absence of Tat.

### BRD2 Suppresses HIV Transcription in the Absence of Tat

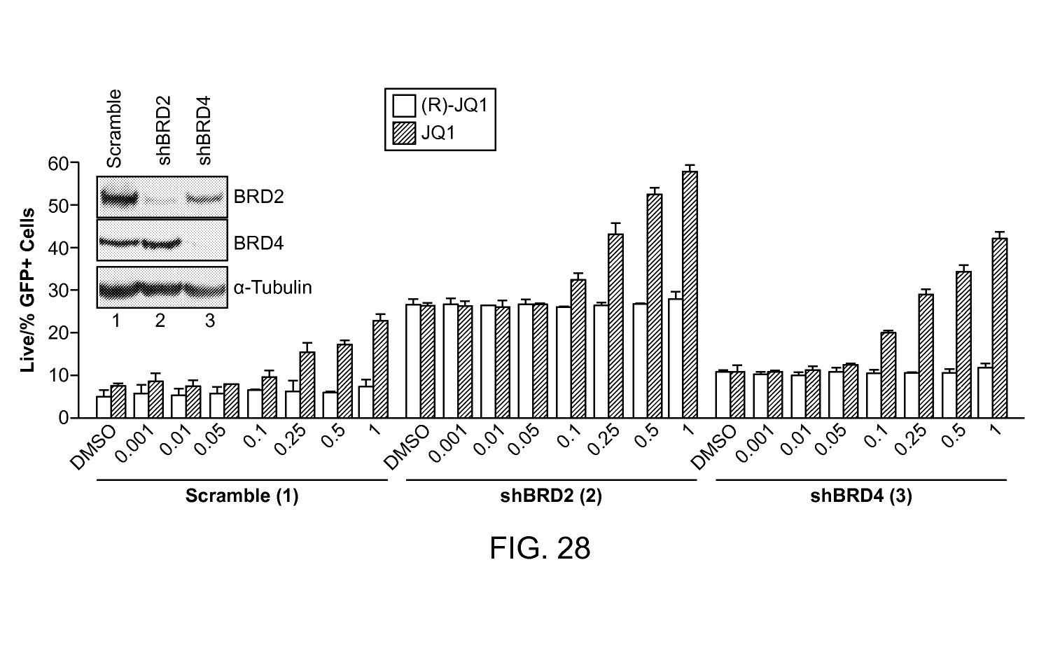

To test the functional relevance of BET proteins in HIV latency, lentiviral shRNA knockdown studies of endogenous BRD2 and BRD4 proteins were performed in A72 cells lacking Tat. BRD2 is a close relative of BRD4, but lacks a C-terminal PID domain. However, in the nuclear complexosome identified by Malovannaya et al. BRD2 was found to coimmunoprecipitate with CDK9/Cyclin T1 or Cyclin T2. BRD2 binds JQ1 and other BET inhibitors, albeit with lower binding affinities than BRD4. Knockdown of BRD2 resulted in a robust activation of the HIV LTR, and this effect was only slightly enhanced in response to JQ1 (twofold as compared to 3.3 fold activation in control cells) (FIG. 25B). BRD4 knockdown also resulted in spontaneous activation of the HIV LTR, albeit to a lesser extent than BRD2 (2.4 fold versus 5.4 fold), and the response to JQ1 was not affected (FIG. 25B). No significant additive or synergistic effects were observed when both factors were knocked down together, indicating that the two factors work in the same biological pathway (FIG. 25B). Similar results were obtained when different sets of shRNAs against BRD2 and BRD4 were used (FIG. 28). These results identify BRD2 as a factor involved in HIV transcription in latent cells.

FIG. 20 demonstrates that JQ1 activates latent HIV. HIV clones R7/E-/GFP and NL4-3/E-/GFP-IRES-nef were derived from pR7-GFP and pNLENG1-EGFP by mutating the Env gene by inserting an early stop codon in the NdeI site. Viral stocks were produced and VSV-G-pseudotyped in 293T cells and titered for p24. Jurkat cells were spininfected with 25 ng of p24 per 10⁶ cells, and GFP⁻ cells where collected in two rounds of cell sorting 5 and 15 days after infection. The expanded population of GFP⁻ cells, composed of uninfected and latently infected cells, was seeded in 96-well plates and treated with the indicated concentration of drugs in duplicates. The percentage of GFP⁺ cells was detected after 24 h at the MACSQuant VYB FACS analyzer. Data are expressed as the mean percentage of GFP⁺ cells, subtracting the average percentage of spontaneous GFP-reactivation in the untreated samples. (A) Jurkat cells containing latent R7/E-/GFP virus were treated with JQ1 or (R)-JQ1 in combination with Prostratin (0.5 μM), SAHA (2.5 μM), TNFα (1 ng/μl), or control at the indicated concentrations for 18 h, followed by flow cytometry analysis. Alone or in combination with Prostratin or TNFα, the BET-inhibitor JQ1, but not the stereoisomer control (R)-JQ1, reactivated HIV-1 in a dose-dependent manner. Similar results were seen in Jurkat cells containing latent NL4-3/E-/GFP-IRES-nef (B). Results represent average of two independent experiments. All treatments in (A) and (B) on the X axis are in μM.

FIG. 21 demonstrates that the JQ1 effect is Tat-independent. Two latent J-Lat cell lines A2 (containing a LTR-Tat-IRES-GFP construct) or A72 (containing a LTR-GFP construct) were treated with JQ1 or (R)-JQ1 in combination with TNFα or control at the indicated concentrations for 18 h, followed by flow cytometry analysis. (A) In A2 cells JQ1, but not the control (R)-JQ1, reactivated HIV-1 in a dose-dependent manner. Similar results were seen in the Tat-deficient A72 Jurkat cell line (B). Data represent average (±SD) of three independent experiments. All treatments in (A) and (B) on the X axis are in μM.

FIG. 22 demonstrates the reactivation of latent HIV with additional bromodomain-targeting compounds. J-Lat cell lines A2 (A) and A72 (B) were treated with JQ1 or two other bromodomain-targeting compounds, I-BET151 and MS417, at the indicated concentrations for 18 h and analyzed by flow cytometry. As indicated, in both A2 and A72 cells, stimulation with all three compounds increased GFP expression. Data represent average (±SD) of three independent experiments.

FIG. 23 demonstrates the effect of bromodomain-targeting compounds in primary T-cell models of HIV latency. (A) Detection of latently infected cells in sorted GFP-negative Bcl-2-transduced cells. The sorted GFP-Bcl-2-transduced resting CD4⁺ T cells were treated with stimuli for 24-72 h at 3TC. Cells treated with 2.5 μg/ml αCD3 and 1 μg/ml αCD28 antibodies were used as positive controls. Reactivation of latent HIV-1 was determined by quantifying % GFP⁺ cells with a MACSQuant flow cytometer (Milteny Biotech GmbH). Results are expressed as percentage of reactivation in response to αCD3 plus αCD28 activation and represent the average of two independent donors. (B) Effect of bromodomain-targeting compounds in latently infected primary nonpolarized T helper cells. Latently infected T cells were generated using healthy, uninfected CD4+T cells (DONOR 144) that were ex vivo differentiated into nonpolarized T cells and infected with DHIV-GFP, X4 virus as previously described.^{49, 50} Reactivation was monitored by analysis of GFP by flow cytometry 96 h after compound addition. Beads coated with αCD3/αCD28 antibodies were used as positive controls. All compounds were tested also in non-infected cells to distinguish between HIV-1 reactivation and compound fluorescence. Similar results were observed in two independent donors.

FIG. 24 demonstrates that JQ1 enhances transcription burst size. (A, B) Depiction of the experimental model with polyclonal LTR-GFP-containing Jurkat cells. (C, D) Cells treated with JQ1 can change the mean expression level of GFP (hypothesis 1), the variability (or coefficient of variation, CV, defined as the standard deviation over the mean; hypothesis 2), or both, compared to the untreated basal expression state. (E) Genome-wide signatures of JQ1 exposure by time-lapse microscopy. Over 2000 cells were accounted for and imaged for durations of 12-18 h. JQ1 displays a similar abundance range with elevated noise magnitude compared to the untreated cell population. (F) Histogram/bar representation of the quantified shifts in burst size (or # of mRNA per pulse, T/k_{off}) and the average dwell time in the OFF state (of 1/kₒₙ) with and without JQ1 treatment. JQ1 increases burst size and 1/kₒₙ.

FIG. 25 demonstrates that the JQ1 effect in A72 cells is dependent on P-TEFb and BRD2. (A) A72 cells were infected with virus containing shRNA constructs targeting cyclin T1 or a nontargeting control. Knockdown of cyclin T1 protein levels are shown by immunoblotting with cyclin T1 or the control α-tubulin antibody. At 4 days after infection, cells were treated with JQ1 or DMSO at the indicated concentrations for 18 h and analyzed by flow cytometry. As indicated, knockdown of Cyclin T1 resulted in a decrease in GFP expression under basal condition and in JQ1-treated cells. Average (±SD) of three experiments is shown. (B) A72 cells were infected with virus containing shRNA constructs targeting BRD2, BRD4 or a nontargeting control. At 4 days after infection, cells were treated with JQ1 or DMSO at the indicated concentrations for 18 h and examined by flow cytometry. As indicated, knockdown of BRD2 and BRD4 resulted in an increase in GFP expression. JQ1 treatment enhanced this effect. Results represent average (±SD) of three experiments. Knockdown of BRD2 and BRD4 protein levels were confirmed by immunoblotting with BRD2 and BRD4 antibodies or the control α-tubulin. All treatments in (A) and (B) on the X axis are in μM.

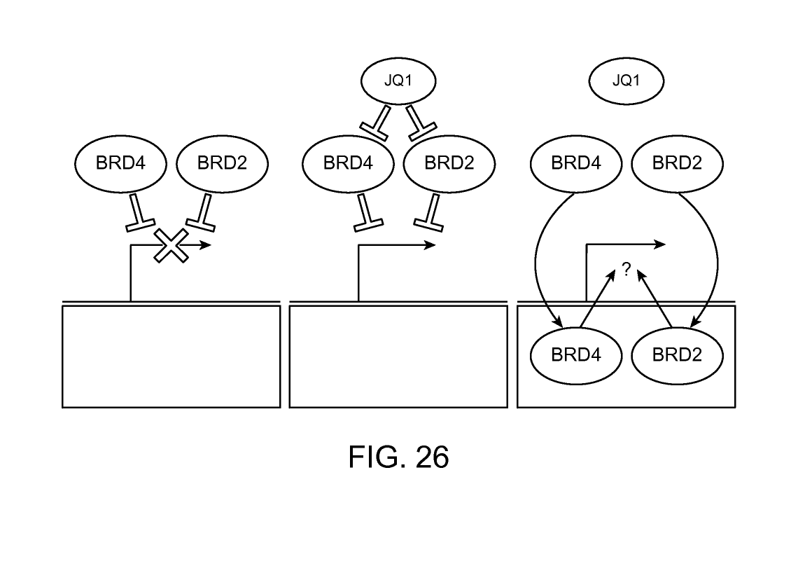

FIG. 26 presents a schematic (model) illustrating that BET proteins restrict HIV transcription in the absence of Tat. JQ1 removes the inhibiting function of BRD2 and BRD4 proteins from latent HIV, a process that may allow both factors to turn into activators of HIV transcription in conjunction with P-TEFb.

FIG. 27 demonstrates that co-treatment with JQ1 and prostratin activates latent HIV. J-Lat cell lines A2 (A) and A72 (B) were treated with JQ1 or (R)-JQ1 in combination with prostratin, SAHA, or control at the indicated concentrations for 18 h, followed by flow cytometry analysis. In both cell lines, JQ1 reactivated HIV-1 in a dose-dependent manner, alone or in combination with prostratin or TNFα. The control (R)-JQ1 did not activate the integrated HIV promoter. Results represent average (±SD) of three experiments. All treatments in (A) and (B) on the X axis are in μM.

FIG. 28 demonstrates the reactivation of latent HIV-1 by inhibition of BRD2 and BRD4. A72 cells were infected with virus containing shRNA constructs targeting different target sequences in BRD2 and BRD4 mRNAs as shown in FIG. 7 or a nontargeting control. Four days after infection, cells were treated with JQ1 or DMSO at the indicated concentrations for 18 h, followed by flow cytometry analysis. As indicated, knockdown of BRD2 and BRD4 resulted in an increase in GFP expression. JQ1 treatment enhanced this effect. Results represent average (±SD) of three experiments. Knockdown of BRD2 and BRD4 protein levels were confirmed by immunoblotting with BRD2 and BRD4 antibodies or the control α-Tubulin. All treatments on the X axis are in μM.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of reactivating latent human immunodeficiency virus (HIV) integrated into the genome of a cell infected with HIV, the method comprising contacting the cell with an agent that binds a bromodomain and reactivates latent HIV integrated into the genome of the cell.

2. The method of claim 1, wherein the agent is a compound of one of Formulas I-XIV.

3. The method of claim 1, wherein the bromodomain is present in a BRD4 polypeptide, a CBP polypeptide, a BRD2 polypeptide, or a PCAF polypeptide.

4. A method of reducing the number of cells containing a latent human immunodeficiency virus in an individual, the method comprising administering to the individual an effective amount of an agent that binds a bromodomain and reactivates latent HIV integrated into the genome of one or more cells in the individual.

5. The method of claim 4, wherein said administering is effective to reduce the number of cells containing a latent human immunodeficiency virus in the individual by at least 20%.

6. The method of claim 4, wherein the agent is a compound of one of Formulas I-XIV.

7. A method of treating a human immunodeficiency virus (HIV) infection in an individual, the method comprising:
administering to an individual an effective amount of a first active agent, wherein the first active agent binds a bromodomain and reactivates latent HIV integrated into the genome of a cell in the individual; and
administering to the individual an effective amount of a second active agent, wherein the second active agent inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

8. The method of claim 7, wherein the first active agent is a compound of one of Formulas I-VI.

9. The method of claim 7, wherein one or both of said administering steps is by a vaginal route of administration, by a rectal route of administration, by an oral route of administration, or by an intravenous route of administration.

10. A drug delivery device comprising:
a) a first container comprising an agent that binds a bromodomain and reactivates latent immunodeficiency virus transcription; and
b) a second container comprising an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

11. The device of claim 10, wherein the agent is a compound of one of Formulas I-XIV.

12. The device of claim 10, wherein the first and second containers are syringes.

13. A method of detecting in a cell sample a cell comprising latent HIV, the method comprising:
a) contacting a cell sample obtained from an individual with an agent that binds a bromodomain and reactivates latent immunodeficiency virus transcription; and
b) detecting HIV gene expression in the cell, compared to HIV gene expression in a control cell not contacted with the agent,
wherein detection of HIV gene expression in the cell contacted with the agent indicates that the cell comprises latent HIV.

14. The method of claim 13, further comprising isolating the cell from the sample.

15. The method of claim 13, wherein detection of HIV gene expression comprises detecting an HIV-encoded gene product.


SEQUENCE LISTING (30 sequences)

SEQ ID NO: 1 - protein, 549 aa, Homo Sapiens
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKK

SEQ ID NO: 2 - protein, 2442 aa, Homo Sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 3 - protein, 832 aa, Homo Sapiens
MSEAGGAGPGGCGAGAGAGAGPGALPPQPAALPPAPPQGSPCAAAAGGSGACGPATAVAAAGTAEGPGGGGSARIAVKKAQLRSAPRAKKLEKLGVYSACKAEESCKCNGWKNPNPSPTPPRADLQQIIVSLTESCRSCSHALAAHVSHLENVSEEEMNRLLGIVLDVEYLFTCVHKEEDADTKQVYFYLFKLLRKSILQRGKPVVEGSLEKKPPFEKPSIEQGVNNFVQYKFSHLPAKERQTIVELAKMFLNRINYWHLEAPSQRRLRSPNDDISGYKENYTRWLCYCNVPQFCDSLPRYETTQVFGRTLLRSVFTVMRRQLLEQARQEKDKLPLEKRTLILTHFPKFLSMLEEEVYSQNSPIWDQDFLSASSRTSQLGIQTVINPPPVAGTISYNSTSSSLEQPNAGSSSPACKASSGLEANPGEKRKMTDSHVLEEAKKPRVMGDIPMELINEVMSTITDPAAMLGPETNFLSAHSARDEAARLEERRGVIEFHVVGNSLNQKPNKKILMWLVGLQNVFSHQLPRMPKEYITRLVFDPKHKTLALIKDGRVIGGICFRMFPSQGFTEIVFCAVTSNEQVKGYGTHLMNHLKEYHIKHDILNFLTYADEYAIGYFKKQGFSKEIKIPKTKYVGYIKDYEGATLMGCELNPRIPYTEFSVIIKKQKEIIKKLIERKQAQIRKVYPGLSCFKDGVRQIPIESIPGIRETGWKPSGKEKSKEPRDPDQLYSTLKSILQQVKSHQSAWPFMEPVKRTEAPGYYEVIRFPMDLKTMSERLKNRYYVSKKLFMADLQRVFTNCKEYNAAESEYYKCANILEKFFFSKIKEAGLIDK

SEQ ID NO: 4 - protein, 107 aa, Artificial sequence
  Synthetic amino acid sequence
RQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINEL

SEQ ID NO: 5 - protein, 101 aa, Artificial sequence
  Synthetic amino acid sequence
LKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRF

SEQ ID NO: 6 - protein, 108 aa, Artificial sequence
  Synthetic amino acid sequence
PEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQ

SEQ ID NO: 7 - protein, 100 aa, Artificial sequence
  Synthetic amino acid sequence
QLYSTLKSILQQVKSHQSAWPFMEPVKRTEAPGYYEVIRFPMDLKTMSERLKNRYYVSKKLFMADLQRVFTNCKEYNAAESEYYKCANILEKFFFSKIKE

SEQ ID NO: 8 - protein, 36 aa, Artificial sequence
  Synthetic amino acid sequence
YTSLIHSLIEESQNQQEKNEQELLELDKWASLWNWF

SEQ ID NO: 9 - protein, 801 aa, Homo Sapiens
MLQNVTPHNKLPGEGNAGLLGLGPEAAAPGKRIRKPSLLYEGFESPTMASVPALQLTPANPPPPEVSNPKKPGRVTNQLQYLHKVVMKALWKHQFAWPFRQPVDAVKLGLPDYHKIIKQPMDMGTIKRRLENNYYWAASECMQDFNTMFTNCYIYNKPTDDIVLMAQTLEKIFLQKVASMPQEEQELVVTIPKNSHKKGAKLAALQGSVTSAHQVPAVSSVSHTALYTPPPEIPTTVLNIPHPSVISSPLLKSLHSAGPPLLAVTAAPPAQPLAKKKGVKRKADTTTPTPTAILAPGSPASPPGSLEPKAARLPPMRRESGRPIKPPRKDLPDSQQQHQSSKKGKLSEQLKHCNGILKELLSKKHAAYAWPFYKPVDASALGLHDYHDIIKHPMDLSTVKRKMENRDYRDAQEFAADVRLMFSNCYKYNPPDHDVVAMARKLQDVFEFRYAKMPDEPLEPGPLPVSTAMPPGLAKSSSESSSEESSSESSSEEEEEEDEEDEEEEESESSDSEEERAHRLAELQEQLRAVHEQLAALSQGPISKPKRKREKKEKKKKRKAEKHRGRAGADEDDKGPRAPRPPQPKKSKKASGSGGGSAALGPSGFGPSGGSGTKLPKKATKTAPPALPTGYDSEEEEESRPMSYDEKRQLSLDINKLPGEKLGRVVHIIQAREPSLRDSNPEEIEIDFETLKPSTLRELERYVLSCLRKKPRKPYTIKKPVGKTKEELALEKKRELEKRLQDVSGQLNSTKKPPKKANEKTESSSAQQVAVSRLSASSSSSDSSSSSSSSSSSDTSDSDSG

SEQ ID NO: 10 - protein, 107 aa, Artificial sequence
  Synthetic amino acid sequence
RVTNQLQYLHKVVMKALWKHQFAWPFRQPVDAVKLGLPDYHKIIKQPMDMGTIKRRLENNYYWAASECMQDFNTMFTNCYIYNKPTDDIVLMAQTLEKIFLQKVASM

SEQ ID NO: 11 - protein, 102 aa, Artificial sequence
  Synthetic amino acid sequence
QLKHCNGILKELLSKKHAAYAWPFYKPVDASALGLHDYHDIIKHPMDLSTVKRKMENRDYRDAQEFAADVRLMFSNCYKYNPPDHDVVAMARKLQDVFEFRY

SEQ ID NO: 12 - protein, 732 aa, Homo Sapiens
MSTATTVAPAGIPATPGPVNPPPPEVSNPSKPGRKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQMPQEEVELLPPAPKGKGRKPAAGAQSAGTQQVAAVSSVSPATPFQSVPPTVSQTPVIAATPVPTITANVTSVPVPPAAAPPPPATPIVPVVPPTPPVVKKKGVKRKADTTTPTTSAITASRSESPPPLSDPKQAKVVARRESGGRPIKPPKKDLEDGEVPQHAGKKGKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMPDEPVFIGREGEAPALPAPAAPMVSKGAESSRSSEESSSDSGSSDSEEERATRLAELQEQLKAVHEQLAALSQAPVNKPKKKKEKKEKEKKKKDKEKEKEKHKVKAEEEKKAKVAPPAKQAQQKKAPAKKANSTTTAGRQLKKGGKQASASYDSEEEEEGLPMSYDEKRQLSLDINRLPGEKLGRVVHIIQSREPSLRDSNPDEIEIDFETLKPTTLRELERYVKSCLQKKQRKPFSASGKKQAAKSKEELAQEKKKELEKRLQDVSGQLSSSKKPARKEKPGSAPSGGPSRLSSSSSSESGSSSSSGSSSDSSDSE

SEQ ID NO: 13 - protein, 107 aa, Artificial sequence
  Synthetic amino acid sequence
RKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQM

SEQ ID NO: 14 - protein, 102 aa, Artificial sequence
  Synthetic amino acid sequence
HLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRF

SEQ ID NO: 15 - protein, 1058 aa, Homo Sapiens
MRRKGRCHRGSAARHPSSPCSVKHSPTRETLTYAQAQRMVEIEIEGRLHRISIFDPLEIILEDDLTAQEMSECNSNKENSERPPVCLRTKRHKNNRVKKKNEALPSAHGTPASASALPEPKVRIVEYSPPSAPRRPPVYYKFIEKSAEELDNEVEYDMDEEDYAWLEIVNEKRKGDCVPAVSQSMFEFLMDRFEKESHCENQKQGEQQSLIDEDAVCCICMDGECQNSNVILFCDMCNLAVHQECYGVPYIPEGQWLCRHCLQSRARPADCVLCPNKGGAFKKTDDDRWGHVVCALWIPEVGFANTVFIEPIDGVRNIPPARWKLTCYLCKQKGVGACIQCHKANCYTAFHVTCAQKAGLYMKMEPVKELTGGGTTFSVRKTAYCDVHTPPGCTRRPLNIYGDVEMKNGVCRKESSVKTVRSTSKVRKKAKKAKKALAEPCAVLPTVCAPYIPPQRLNRIANQVAIQRKKQFVERAHSYWLLKRLSRNGAPLLRRLQSSLQSQRSSQQRENDEEMKAAKEKLKYWQRLRHDLERARLLIELLRKREKLKREQVKVEQVAMELRLTPLTVLLRSVLDQLQDKDPARIFAQPVSLKEVPDYLDHIKHPMDFATMRKRLEAQGYKNLHEFEEDFDLIIDNCMKYNARDTVFYRAAVRLRDQGGVVLRQARREVDSIGLEEASGMHLPERPAAAPRRPFSWEDVDRLLDPANRAHLGLEEQLRELLDMLDLTCAMKSSGSRSKRAKLLKKEIALLRNKLSQQHSQPLPTGPGLEGFEEDGAALGPEAGEEVLPRLETLLQPRKRSRSTCGDSEVEEESPGKRLDAGLTNGFGGARSEQEPGGGLGRKATPRRRCASESSISSSNSPLCDSSFNAPKCGRGKPALVRRHTLEDRSELISCIENGNYAKAARIAAEVGQSSMWISTDAAASVLEPLKVVWAKCSGYPSYPALIIDPKMPRVPGHHNGVTIPAPPLDVLKIGEHMQTKSDEKLFLVLFFDNKRSWQWLPKSKMVPLGIDETIDKLKMMEGRNSSIRKAVRIAFDRAMNHLSRVHGEPTSDLSDID

SEQ ID NO: 16 - protein, 98 aa, Artificial sequence
  Synthetic amino acid sequence
PLTVLLRSVLDQLQDKDPARIFAQPVSLKEVPDYLDHIKHPMDFATMRKRLEAQGYKNLHEFEEDFDLIIDNCMKYNARDTVFYRAAVRLRDQGGVVL

SEQ ID NO: 17 - protein, 947 aa, Homo Sapiens
MSLPSRQTAIIVNPPPPEYINTKKNGRLTNQLQYLQKVVLKDLWKHSFSWPFQRPVDAVKLQLPDYYTIIKNPMDLNTIKKRLENKYYAKASECIEDFNTMFSNCYLYNKPGDDIVLMAQALEKLFMQKLSQMPQEEQVVGVKERIKKGTQQNIAVSSAKEKSSPSATEKVFKQQEIPSVFPKTSISPLNVVQGASVNSSSQTAAQVTKGVKRKADTTTPATSAVKASSEFSPTFTEKSVALPPIKENMPKNVLPDSQQQYNVVKTVKVTEQLRHCSEILKEMLAKKHFSYAWPFYNPVDVNALGLHNYYDVVKNPMDLGTIKEKMDNQEYKDAYKFAADVRLMFMNCYKYNPPDHEVVTMARMLQDVFETHFSKIPIEPVESMPLCYIKTDITETTGRENTNEASSEGNSSDDSEDERVKRLAKLQEQLKAVHQQLQVLSQVPFRKLNKKKEKSKKEKKKEKVNNSNENPRKMCEQMRLKEKSKRNQPKKRKQQFIGLKSEDEDNAKPMNYDEKRQLSLNINKLPGDKLGRVVHIIQSREPSLSNSNPDEIEIDFETLKASTLRELEKYVSACLRKRPLKPPAKKIMMSKEELHSQKKQELEKRLLDVNNQLNSRKRQTKSDKTQPSKAVENVSRLSESSSSSSSSSESESSSSDLSSSDSSDSESEMFPKFTEVKPNDSPSKENVKKMKNECILPEGRTGVTQIGYCVQDTTSANTTLVHQTTPSHVMPPNHHQLAFNYQELEHLQTVKNISPLQILPPSGDSEQLSNGITVMHPSGDSDTTMLESECQAPVQKDIKIKNADSWKSLGKPVKPSGVMKSSDELFNQFRKAAIEKEVKARTQELIRKHLEQNTKELKASQENQRDLGNGLTVESFSNKIQNKCSGEEQKEHQQSSEAQDKSKLWLLKDRDLARQKEQERRRREAMVGTIDMTLQSDIMTMFENNFD

SEQ ID NO: 18 - protein, 107 aa, Artificial sequence
  Synthetic amino acid sequence
RLTNQLQYLQKVVLKDLWKHSFSWPFQRPVDAVKLQLPDYYTIIKNPMDLNTIKKRLENKYYAKASECIEDFNTMFSNCYLYNKPGDDIVLMAQALEKLFMQKLSQM

SEQ ID NO: 19 - protein, 102 aa, Artificial sequence
  Synthetic amino acid sequence
QLRHCSEILKEMLAKKHFSYAWPFYNPVDVNALGLHNYYDVVKNPMDLGTIKEKMDNQEYKDAYKFAADVRLMFMNCYKYNPPDHEVVTMARMLQDVFETHF

SEQ ID NO: 20 - protein, 652 aa, Homo Sapiens
MGKKHKKHKSDKHLYEEYVEKPLKLVLKVGGNEVTELSTGSSGHDSSLFEDKNDHDKHKDRKRKKRKKGEKQIPGEEKGRKRRRVKEDKKKRDRDRVENEAEKDLQCHAPVRLDLPPEKPLTSSLAKQEEVEQTPLQEALNQLMRQLQRKDPSAFFSFPVTDFIAPGYSMIIKHPMDFSTMKEKIKNNDYQSIEELKDNFKLMCTNAMIYNKPETIYYKAAKKLLHSGMKILSQERIQSLKQSIDFMADLQKTRKQKDGTDTSQSGEDGGCWQREREDSGDAEAHAFKSPSKENKKKDKDMLEDKFKSNNLEREQEQLDRIVKESGGKLTRRLVNSQCEFERRKPDGTTTLGLLHPVDPIVGEPGYCPVRLGMTTGRLQSGVNTLQGFKEDKRNKVTPVLYLNYGPYSSYAPHYDSTFANISKDDSDLIYSTYGEDSDLPSDFSIHEFLATCQDYPYVMADSLLDVLTKGGHSRTLQEMEMSLPEDEGHTRTLDTAKEMEQITEVEPPGRLDSSTQDRLIALKAVTNFGVPVEVFDSEEAEIFQKKLDETTRLLRELQEAQNERLSTRPPPNMICLLGPSYREMHLAEQVTNNLKELAQQVTPGDIVSTYGVRKAMGISIPSPVMENNFVDLTEDTEEPKKTDVAECGPGGS

SEQ ID NO: 21 - protein, 98 aa, Artificial sequence
  Synthetic amino acid sequence
PLQEALNQLMRQLQRKDPSAFFSFPVTDFIAPGYSMIIKHPMDFSTMKEKIKNNDYQSIEELKDNFKLMCTNAMIYNKPETIYYKAAKKLLHSGMKIL

SEQ ID NO: 22 - protein, 651 aa, Homo Sapiens
MGKKHKKHKSDKHLYEEYVEKPLKLVLKVGGNEVTELSTGSSGHDSSLFEDKNDHDKHKDRKRKKRKKGEKQIPGEEKGRKRRRVKEDKKKRDRDRVENEAEKDLQCHAPVRLDLPPEKPLTSSLAKQEEVEQTPLQEALNQLMRQLQRKDPSAFFSFPVTDFIAPGYSMIIKHPMDFSTMKEKIKNNDYQSIEELKDNFKLMCTNAMIYNKPETIYYKAAKKLLHSGMKILSQERIQSLKQSIDFMADLQKTRKQKDGTDTSQSGEDGGCWQREREDSGDAEAHAFKSPSKENKKKDKDMLEDKFKSNNLEREQEQLDRIVKESGGKLTRRLVNSQCEFERRKPDGTTTLGLLHPVDPIVGEPGYCPVRLGMTTGRLQSGVNTLQGFKEDKRNKVTPVLYLNYGPYSSYAPHYDSTFANISKDDSDLIYSTYGEDSDLPSDFSIHEFLATCQDYPYVMADSLLDVLTKGGHSRTLQEMEMSLPEDEGHTRTLDTAKEMEITEVEPPGRLDSSTQDRLIALKAVTNFGVPVEVFDSEEAEIFQKKLDETTRLLRELQEAQNERLSTRPPPNMICLLGPSYREMHLAEQVTNNLKELAQQVTPGDIVSTYGVRKAMGISIPSPVMENNFVDLTEDTEEPKKTDVAECGPGGS

SEQ ID NO: 23 - protein, 957 aa, Homo Sapiens
MATGTGKHKLLSTGPTEPWSIREKLCLASSVMRSGDQNWVSVSRAIKPFAEPGRPPDWFSQKHCASQYSELLETTETPKRKRGEKGEVVETVEDVIVRKLTAERVEELKKVIKETQERYRRLKRDAELIQAGHMDSRLDELCNDIATKKKLEEEEAEVKRKATDAAYQARQAVKTPPRRLPTVMVRSPIDSASPGGDYPLGDLTPTTMEEATSGVTPGTLPSTPVTSFPGIPDTLPPGSAPLEAPMTPVTDDSPQKKMLGQKATPPPSPLLSELLKKGSLLPTSPRLVNESEMAVASGHLNSTGVLLEVGGVLPMIHGGEIQQTPNTVAASPAASGAPTLSRLLEAGPTQFTTPLASFTTVASEPPVKLVPPPVESVSQATIVMMPALPAPSSAPAVSTTESVAPVSQPDNCVPMEAVGDPHTVTVSMDSSEISMIINSIKEECFRSGVAEAPVGSKAPSIDGKEELDLAEKMDIAVSYTGEELDFETVGDIIAIIEDKVDDHPEVLDVAAVEAALSFCEENDDPQSLPGPWEHPIQQERDKPVPLPAPEMTVKQERLDFEETENKGIHELVDIREPSAEIKVEPAEPEPVISGAEIVAGVVPATSMEPPELRSQDLDEELGSTAAGEIVEADVAIGKGDETPLTNVKTEASPESMLSPSHGSNPIEDPLEAETQHKFEMSDSLKEESGTIFGSQIKDAPGEDEEEDGVSEAASLEEPKEEDQGEGYLSEMDNEPPVSESDDGFSIHNATLQSHTLADSIPSSPASSQFSVCSEDQEAIQAQKIWKKAIMLVWRAAANHRYANVFLQPVTDDIAPGYHSIVQRPMDLSTIKKNIENGLIRSTAEFQRDIMLMFQNAVMYNSSDHDVYHMAVEMQRDVLEQIQQFLATQLIMQTSESGISAKSLRGRDSTRKQDASEKDSVPMGSPAFLLSLFDGGTRGRRCAIEADMKMKKSEQIDN

SEQ ID NO: 24 - protein, 104 aa, Artificial sequence
  Synthetic amino acid sequence
QKIWKKAIMLVWRAAANHRYANVFLQPVTDDIAPGYHSIVQRPMDLSTIKKNIENGLIRSTAEFQRDIMLMFQNAVMYNSSDHDVYHMAVEMQRDVLEQIQQFL

SEQ ID NO: 25 - protein, 1241 aa, Homo Sapiens
MATGTGKHKLLSTGPTEPWSIREKLCLASSVMRSGDQNWVSVSRAIKPFAEPGRPPDWFSQKHCASQYSELLETTETPKRKRGEKGEVVETVEDVIVRKLTAERVEELKKVIKETQERYRRLKRDAELIQAGHMDSRLDELCNDIATKKKLEEEEAEVKRKATDAAYQARQAVKTPPRRLPTVMVRSPIDSASPGGDYPLGDLTPTTMEEATSGVNESEMAVASGHLNSTGVLLEVGGVLPMIHGGEIQQTPNTVAASPAASGAPTLSRLLEAGPTQFTTPLASFTTVASEPPVKLVPPPVESVSQATIVMMPALPAPSSAPAVSTTESVAPVSQPDNCVPMEAVGDPHTVTVSMDSSEISMIINSIKEECFRSGVAEAPVGSKAPSIDGKEELDLAEKMDIAVSYTGEELDFETVGDIIAIIEDKVDDHPEVLDVAAVEAALSFCEENDDPQSLPGPWEHPIQQERDKPVPLPAPEMTVKQERLDFEETENKGIHELVDIREPSAEIKVEPAEPEPVISGAEIVAGVVPATSMEPPELRSQDLDEELGSTAAGEIVEADVAIGKGDETPLTNVKTEASPESMLSPSHGSNPIEDPLEAETQHKFEMSDSLKEESGTIFGSQIKDAPGEDEEEDGVSEAASLEEPKEEDQGEGYLSEMDNEPPVSESDDGFSIHNATLQSHTLADSIPSSPASSQFSVCSEDQEAIQAQKIWKKAIMLVWRAAANHRYANVFLQPVTDDIAPGYHSIVQRPMDLSTIKKNIENGLIRSTAEFQRDIMLMFQNAVMYNSSDHDVYHMAVEMQRDVLEQIQQFLATQLIMQTSESGISAKSLRGRDSTRKQDASEKDSVPMGSPAFLLSLFMGHEWVWLDSEQDHPNDSELSNDCRSLFSSWDSSLDLDVGNWRETEDPEAEELEESSPEREPSELLVGDGGSEESQEAARKASHQNLLHFLSEVAYLMEPLCISSNESSEGCCPPSGTRQEGREIKASEGERELCRETEELSAKGDPLVAEKPLGENGKPEVASAPSVICTVQGLLTESEEGEAQQESKGEDQGEVYVSEMEDQPPSGECDDAFNIKETPLVDTLFSHATSSKLTDLSQDDPVQDHLLFKKTLLPVWKMIASHRFSSPFLKPVSERQAPGYKDVVKRPMDLTSLKRNLSKGRIRTMAQFLRDLMLMFQNAVMYNDSDHHVYHMAVEMRREVLEQIQVLNIWLDKRKGSSSLEGEPANPVDDGKPVFSEQIDN

SEQ ID NO: 26 - protein, 866 aa, Homo Sapiens
MATGTGKHKLLSTGPTEPWSIREKLCLASSVMRSGDQNWVSVSRAIKPFAEPGRPPDWFSQKHCASQYSELLETTETPKRKRGEKGEVVETVEDVIVRKLTAERVEELKKVIKETQERYRRLKRDAELIQAGHMDSRLDELCNDIATKKKLEEEEAEVKRKATDAAYQARQAVKTPPRRLPTVMVRSPIDSASPGGDYPLGDLTPTTMEEATSGVTPGTLPSTPVTSFPGIPDTLPPGSAPLEAPMTPVTDDSPQKKMLGQKATPPPSPLLSELLKKGSLLPTSPRLVNESEMAVASGHLNSTGVLLEVGGVLPMIHGGEIQQTPNTVAASPAASVSQPDNCVPMEAVGDPHTVTVSMDSSEISMIINSIKEECFRSGVAEAPVGSKAPSIDGKEELDLAEKMDIAVSYTGEELDFETVGDIIAIIEDKVDDHPEVLDVAAVEAALSFCEENDDPQSLPGPWEHPIQQERDKPVPLPAPEMTVKQERLDFEETENKGIHELVDIREPSAEIKVEPAEPEPVISGAEIVAGVVPATSMEPPELRSQDLDEELGSTAAGEIVEADVAIGKGDETPLTNVKTEASPESMLSPSHGSNPIEDPLEAETQHKFEMSDSLKEESGTIFGSQIKDAPGEDEEEDGVSEAASLEEPKEEDQGEGYLSEMDNEPPVSESDDGFSIHNATLQSHTLADSIPSSPASSQFSVCSEDQEAIQAQKIWKKAIMLVWRAAANHRYANVFLQPVTDDIAPGYHSIVQRPMDLSTIKKNIENGLIRSTAEFQRDIMLMFQNAVMYNSSDHDVYHMAVEMQRDVLEQIQQFLATQLIMQTSESGISAKSLRGRDSTRKQDASEKDGGTRGRRCAIEADMKMKK

SEQ ID NO: 27 - protein, 597 aa, Homo Sapiens
MGKKHKKHKAEWRSSYEDYADKPLEKPLKLVLKVGGSEVTELSGSGHDSSYYDDRSDHERERHKEKKKKKKKKSEKEKHLDDEERRKRKEEKKRKREREHCDTEGEADDFDPGKKVEVEPPPDRPVRACRTQPAENESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSKQAALLGNEDTAVEEPVPEVVPVQVETAKKSKKPSREVISCMFEPEGNACSLTDSTAEEHVLALVEHAADEARDRINRFLPGGKMGYLKRNGDGSLLYSVVNTAEPDADEEETHPVDLSSLSSKLLPGFTTLGFKDERRNKVTFLSSATTALSMQNNSVFGDLKSDEMELLYSAYGDETGVQCALSLQEFVKDAGSYSKKVVDDLLDQITGGDHSRTLFQLKQRRNVPMKPPDEAKVGDTLGDSSSSVLEFMSMKSYPDVSVDISMLSSLGKVKKELDPDDSHLNLDETTKLLQDLHEAQAERGGSRPSSNLSSLSNASERDQHHLGSPSRLSVGEQPDVTHDPYEFLQSPEPAASAKT

SEQ ID NO: 28 - protein, 98 aa, Artificial sequence
  Synthetic amino acid sequence
PIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMM

SEQ ID NO: 29 - protein, 550 aa, Homo Sapiens
MMTGQTMSERGTKKRKRRRRRSPRRRSIWTMRKEGSERKRRSGSERGSTVTRRERLTTLILGRRWRWSRPQIGQSERAGHSQPKMRAHLFSNSWNTSSASFRDPHGFFAFPVTDAIAPGYSMIIKHPMDFGTMKDKIVANEYKSVTEFKADFKLMCDNAMTYNRPDTVYYKLAKKILHAGFKMMSKQAALLGNEDTAVEEPVPEVVPVQVETAKKSKKPSREVISCMFEPEGNACSLTDSTAEEHVLALVEHAADEARDRINRFLPGGKMGYLKRNGDGSLLYSVVNTAEPDADEEETHPVDLSSLSSKLLPGFTTLGFKDERRNKVTFLSSATTALSMQNNSVFGDLKSDEMELLYSAYGDETGVQCALSLQEFVKDAGSYSKKVVDDLLDQITGGDHSRTLFQLKQRRNVPMKPPDEAKVGDTLGDSSSSVLEFMSMKSYPDVSVDISMLSSLGKVKKELDPDDSHLNLDETTKLLQDLHEAQAERGGSRPSSNLSSLSNASERDQHHLGSPSRLSVGEQPDVTHDPYEFLQSPEPAASAKTSEQIDN

SEQ ID NO: 30 - protein, 83 aa, Artificial sequence
  Synthetic amino acid sequence
RDPHGFFAFPVTDAIAPGYSMIIKHPMDFGTMKDKIVANEYKSVTEFKADFKLMCDNAMTYNRPDTVYYKLAKKILHAGFKMM